SEQUENCE LISTING (94 sequences)

SEQ ID NO: 1 - DNA, 326 bp, Homo sapiens
gttttttttttgcagtgtactaaaattgtgttcaaagtttatttcaagtcacagaaaatatacaaactaagacaataaaatgaagtgattttgttctgagagaaggtgatgggagtcctccacctggcactgatttgagtcattcaagggttaggatgcaggtcatcttttttcaatatttcaacaaaattatagaatttccagatttcttgccaggtatttttgaaaacctatcaaggtgctccattggatttctgtatttgttcctttaaaatcatggctactcctgcctttggctcatggtaggcaaacatggcaatcctggt

SEQ ID NO: 2 - DNA, 1978 bp, Homo sapiens
cggaagccgactcaacagagctatggcgggtttgactgtgagagacccagcggtggatcgttctctacgttctgtgttcgtggggaacattccttatgaagctactgaagagcagttgaaggacatcttttctgaggttggacctgttgttagtttcagattggtatacgatagagagacaggaaagccaaagggttatggcttctgtgaataccaagaccaagagacagcacttagtgccatgcggaacctgaatgggcgcgaattcagtgggagagcacttcgagtggacaatgctgccagtgaaaagaacaaagaagagctgaagagccttggcactggtgcccctgtcattgagtcaccttatggagagaccatcagtcctgaggatgcccctgagtccattagcaaagcagttgccagccttccaccagagcagatgtttgagctgatgaaacaaatgaagctctgtgtccagaatagtccccaggaggcacggaacatgttacttcagaaccctcaactggcttatgctttgctgcaagcacaggtagtgatgagaattgtggatccggaaattgccctgaaaattctgcatcgccagacaaatatcccaacgctgattgcaggcaaccctcagccagtccatggtgctgggcctggctcaggatccaatgtgtcaatgaaccagcagaatcctcaggcccctcaggcccagtctttgggtggaatgcatgtcaatggcgcacctcctctgatgcaagcttctatgcagggtggagttccagcaccagggcaaatgccagctgctgtcacaggacctggccctggttccttagctcctggaggaggaatgcaggctcaggttggaatgccaggaagtggaccagtgtccatggaacgggggcaagtgccgatgcaagaccccagagcagctatgcagcggggatccttgcctgcgaatgtcccaacccctcgaggcttgttaggagatgctccgaatgatccacggggaggcactttactttctgtaactggagaggtagagcctagaggttacttgggaccacctcatcagggtccacccatgcaccatgtccctggccatgagagccgaggaccacccccacatgaactgaggggagggccattacccgagcccagacctctaatggcagaaccaagaggacccatgctagatcagaggggtccacccttggatggcagaggtggaagggatccccgaggaatagatgcacgagggatggaggcccgagccatggaggcaagagggttagatgccagaggattagaggcccgtgcaatggaggcccgtgcgatggaagctcgtgcaatggaggcccgagcgatggaggcccgtgcaatggaagtccgagggatggaggccagaggcatggataccagaggcccagtgcctggccccagaggacctatacctagtggaatgcagggtcccagtccaattaacatgggggcggttgtcccccagggatccagacaggtcccagtcatgcagggaacaggaatgcaaggagcaagtatacagggtggaagccagcctggcggctttagtcccgggcagaaccaagtcactccacaggatcatgagaaggctgctttgattatgcaggttctacaactgactgcagaccagattgccatgttgcctcctgagcaaaggcagagtatcctgattttaaaggaacaaatacagaaatccactggagcaccttgataggttttcaaaaatacctggcaagaaatctggaaattctataattttgttgaaatattgaaaaaagatgacctgcatcctaacccttgaatgactcaaatcagtgccaggtggaggactcccatcaccttctctcagaacaaaatcacttcattttattgtcttagtttgtatattttctgtgacttgaaataaactttgaacacaattttagtacactgc

SEQ ID NO: 3 - DNA, 213 bp, Homo sapiens
gcaaaccaaaaactagtttacaatactgaagttataggaaacaagcacaaaaggccattgggtggggctggatggagcatgatctggtccttaaagtatgcagatcgcctgggaggcccagcctatggagattgggaggagagggagatgctggtgactggagatggtgagagcctgggctggggtagtttttagtcatgggcttatggaccc

SEQ ID NO: 4 - DNA, 3084 bp, Homo sapiens
tcggggagagaagctggactgcagctggtttcaggaacttctcttgacgagaagagagaccaaggaggccaagcaggggctgggccagaggtgccaacatggggaaactgaggctcggctcggaaaggtgaagtaacttgtccaagatcacaaagctggtgaacatcaagttggtgctatggcaaggctgggaaactgcagcctgacttgggctgccctgatcatcctgctgctccccggaagtctggaggagtgcgggcacatcagtgtctcagcccccatcgtccacctgggggatcccatcacagcctcctgcatcatcaagcagaactgcagccatctggacccggagccacagattctgtggagactgggagcagagcttcagcccgggggcaggcagcagcgtctgtctgatgggacccaggaatctatcatcaccctgccccacctcaaccacactcaggcctttctctcctgctgcctgaactggggcaacagcctgcagatcctggaccaggttgagctgcgcgcaggctaccctccagccataccccacaacctctcctgcctcatgaacctcacaaccagcagcctcatctgccagtgggagccaggacctgagacccacctacccaccagcttcactctgaagagtttcaagagccggggcaactgtcagacccaaggggactccatcctggactgcgtgcccaaggacgggcagagccactgctgcatcccacgcaaacacctgctgttgtaccagaatatgggcatctgggtgcaggcagagaatgcgctggggaccagcatgtccccacaactgtgtcttgatcccatggatgttgtgaaactggagccccccatgctgcggaccatggaccccagccctgaagcggcccctccccaggcaggctgcctacagctgtgctgggagccatggcagccaggcctgcacataaatcagaagtgtgagctgcgccacaagccgcagcgtggagaagccagctgggcactggtgggccccctccccttggaggcccttcagtatgagctctgcgggctcctcccagccacggcctacaccctgcagatacgctgcatccgctggcccctgcctggccactggagcgactggagccccagcctggagctgagaactaccgaacgggcccccactgtcagactggacacatggtggcggcagaggcagctggaccccaggacagtgcagctgttctggaagccagtgcccctggaggaagacagcggacggatccaaggttatgtggtttcttggagaccctcaggccaggctggggccatcctgcccctctgcaacaccacagagctcagctgcaccttccacctgccttcagaagcccaggaggtggcccttgtggcctataactcagccgggacctctcgtcccactccggtggtcttctcagaaagcagaggcccagctctgaccagactccatgccatggcccgagaccctcacagcctctgggtaggctgggagccccccaatccatggcctcagggctatgtgattgagtggggcctgggcccccccagcgcgagcaatagcaacaagacctggaggatggaacagaatgggagagccacggggtttctgctgaaggagaacatcaggccctttcagctctatgagatcatcgtgactcccttgtaccaggacaccatgggaccctcccagcatgtctatgcctactctcaagaaatggctccctcccatgccccagagctgcatctaaagcacattggcaagacctgggcacagctggagtgggtgcctgagccccctgagctggggaagagcccccttacccactacaccatcttctggaccaacgctcagaaccagtccttctccgccatcctgaatgcctcctcccgtggctttgtcctccatggcctggagcccgccagtctgtatcacatccacctcatggctgccagccaggctggggccaccaacagtacagtcctcaccctgatgaccttgaccccagaggggtcggagctacacatcatcctgggcctgttcggcctcctgctgttgctcacctgcctctgtggaactgcctggctctgttgcagccccaacaggaagaatcccctctggccaagtgtcccagacccagctcacagcagcctgggctcctgggtgcccacaatcatggaggagctgcccggacccagacagggacagtggctggggcagacatctgaaatgagccgtgctctcaccccacatccttgtgtgcaggatgccttccagctgcccggccttggcacgccacccatcaccaagctcacagtgctggaggaggatgaaaagaagccggtgccctgggagtcccataacagctcagagacctgtggcctccccactctggtccagacctatgtgctccagggggacccaagagcagtttccacccagccccaatcccagtctggcaccagcgatcaggtcctttatgggcagctgctgggcagccccacaagcccagggccagggcactatctccgctgtgactccactcagcccctcttggcgggcctcacccccagccccaagtcctatgagaacctctggttccaggccagccccttggggaccctggtaaccccagccccaagccaggaggacgactgtgtctttgggccactgctcaacttccccctcctgcaggggatccgggtccatgggatggaggcgctggggagcttctagggcttcctggggttcccttcttgggcctgcctcttaaaggcctgagctagctggagaagaggggagggtccataagcccatgactaaaaactaccccagcccaggctctcaccatctccagtcaccagcatctccctctcctcccaatctccataggctgggcctcccaggcgatctgcatactttaaggaccagatcatgctccatccagccccacccaatggccttttgtgcttgtttcctataacttcagtattgtaaactagtttttggtttgcagtttttgttgttgtttatagacactcttgggtgta

SEQ ID NO: 5 - DNA, 252 bp, Homo sapiens
tttttttttttttttttgctcacctgagtattttgctttattcaatctaataaacattttatttatgtaaaagacaaacaatgcatagaataaaaataagtgcttgagacttttgatataaaaagagtatatagcattcacattcgtattttaatacatgagtacagctgaagtgttccataaaagaataaaactttccctttatgtatagtagtgaaaaaagtcagtatttttaggaactacagaatgtta

SEQ ID NO: 6 - DNA, 5759 bp, Homo sapiens
ataccctgtggtgctagtgtaaaatcaaatataatctacctctttagcttttctgttatgtaattggaatgaaaaaatagtgcttttttttttttttcatttttgagattgagttaccatggagggaaagaacatgtgttttaccagtggaacgcattctttgtgttatctcttcgatttttaacattttatgtttccagtttataaactttgcctccagtttgatgaagaaaatttagctattagaaagtatttaaaatctcaaaagtatgacattttaaaatgttagcaggtttaaagataacttagtttttcaactcttaacccaaaattatacttgtatactataatttgtgttgaattacttggtattatttatcatcctagtaagatctattttatataatttgattcactttttatattgaattatgttaattgtttttgtaatacacctttcatatttcttcaaagaaaactagtgttgggttattgctaattttgcttcgtggtttggttatttacatataaaaattatattgttttaaaaattgcaaagtgatcatttattgacacatcagcaagaattaatgttgaaatgcaagttttttcccctatccttaactaagtgtgtagatatctttgttttgtttctttaatagatagcaaacaataaagatgcattgaggaagacatggaaccctaagtttacattgagaagtcactttgatggcatccgagcccttgctttccatcccattgagcctgttttgataacagcatcagaggatcacacattaaaaatgtggaatttacagaaaacagccccagccaaaaagtgagaatattctactttaacattatttgaatattttaagtaacattttcagcagaaaactacatattttatgatttctgttaaatttcccaaacatttcttctagttctttcttccatgtcatgtaatttattgtgtttttccatttatacaacctaatgctaggttagaaaactgatgcttaggaagatatggtattatcttttggtttcaccagggtttgtgaatactgattacagtctttatacataacagtatttttaaatggctgctatatttggggagccccttgtctatactgaaggtttggactgttcttgtaaacaggtatatgcatctttaaaataatcaaatgtccacctccaccttgccccattgtttactagatttagacttatgagatggagcagagaaaagcctcactgggatgggtggagggtcttggtcttgtcttttggccttgagactttaggtactttccatacccaccaatatgtctagactcagaggctaacatgactgctagtgctcaccttggccctttagttcctgtctaagcatggatagacatgagttagtataaaccctttaaaagtcacaaagctaaatctggttctacttgcttgggtccttatcggagctccactcctttgggtcacatgtcaaaaaaaaaaaaaaaaaggcgccgtccgctgcgctgggggctcggtctatgacgagcagcggggtctgccatgggtcgggggctgctcaggggcctgtggccgctgcacatcgtcctgtggacgcgtatcgccagcacgatcccaccgcacgttcagaagtcggttaataacgacatgatagtcactgacaacaacggtgcagtcaagtttccacaactgtgtaaattttgtgatgtgagattttccacctgtgacaaccagaaatcctgcatgagcaactgcagcatcacctccatctgtgagaagccacaggaagtctgtgtggctgtatggagaaagaatgacgagaacataacactagagacagtttgccatgaccccaagctcccctaccatgactttattctggaagatgctgcttctccaaagtgcattatgaaggaaaaaaaaaagcctggtgagactttcttcatgtgttcctgtagctctgatgagtgcaatgacaacatcatcttctcagaagaatataacaccagcaatcctgacttgttgctagtcatatttcaagtgacaggcatcagcctcctgccaccactgggagttgccatatctgtcatcatcatcttctactgctaccgcgttaaccggcagcagaagctgagttcaacctgggaaaccggcaagacgcggaagctcatggagttcagcgagcactgtgccatcatcctggaagatgaccgctctgacatcagctccacgtgtgccaacaacatcaaccacaacacagagctgctgcccattgagctggacaccctggtggggaaaggtcgctttgctgaggtctataaggccaagctgaagcagaacacttcagagcagtttgagacagtggcagtcaagatctttccctatgaggagtatgcctcttggaagacagagaaggacatcttctcagacatcaatctgaagcatgagaacatactccagttcctgacggctgaggagcggaagacggagttggggaaacaatactggctgatcaccgccttccacgccaagggcaacctacaggagtacctgacgcggcatgtcatcagctgggaggacctgcgcaagctgggcagctccctcgcccgggggattgctcacctccacagtgatcacactccatgtgggaggcccaagatgcccatcgtgcacagggacctcaacagctccaatatcctcgtgaagaacgacctaacctgctgcctgtgtgactttgggctttccctgcgtctggaccctactctgtctgtggatgacctggctaacagtgggcaggtgggaactgcaagatacatggctccagaagtcctagaatccaggatgaatttggagaatgctgagtccttcaagcagaccgatgtctactccatggctctggtgctctgggaaatgacatctcgctgtaatgcagtgggagaagtaaaagattatgagcctccatttggttccaaggtgcgggagcacccctgtgtcgaaagcatgaaggacaacgtgttgagagatcgagggcgaccagaaattcccagcttctggctcaaccaccagggcatccagatggtgtgtgagacgttgactgagtgctgggaccacgacccagaggcccgtctcacagcccagtgtgtggcagaacgcttcagtgagctggagcatctggacaggctctcggggaggagctgctcggaggagaagattcctgaagacggctccctaaacactaccaaatagctcttctggggcaggctgggccatgtccaaagaggctgcccctctcaccaaagaacagaggcagcaggaagctgcccctgaactgatgcttcctggaaaaccaagggggtcactcccctccctgtaagctgtggggataagcagaaacaacagcagcagggagtgggtgacatagagcattctatgccttttacattgtcataggataagctgtgttagcacttcctcaggaaatgagattgattttacaatagccaataacatttgcactttattaatgcctgtatataaatatgaatagctatgttttatatatatatatatatatctatatatgtctatagctctatatatatagccataccttgaaaagagacaaggaaaaacatcaaatattcccaggaaattggttttattggagaactccagaaccaagcagagaaggaagggacccatgacagcattagcatttgacaatcacacatgcagtggttctctgactgtaaaacagtgaactttgcatgaggaaagaggctccatgtctcacagccagctatgaccacattgcacttgcttttgcaaaataatcattccctgcctagcacttctcttctggccatggaactaagtacagtggcactgtttgaggaccagtgttcccggggttcctgtgtgcccttatttctcctggacttttcatttaagctccaagccccaaatctggggggctagtttagaaactctccctcaacctagtttagaaactctaccccatctttaataccttgaatgttttgaaccccactttttaccttcatgggttgcagaaaaatcagaacagatgtccccatccatgcgattgccccaccatctactaatgaaaaattgttctttttttcatctttcccctgcacttatgttactattctctgctcccagccttcatccttttctaaaaaggagcaaattctcactctaggctttatcgtgtttactttttcattacacttgacttgattttctagttttctatacaaacaccaatgggttccatctttctgggctcctgattgctcaagcacagtttggcctgatgaagaggatttcaactacacaatactatcattgtcaggactatgacctcaggcactctaaacatatgttttgtttggtcagcacagcgtttcaaaaagtgaagccactttataaatatttggagattttgcaggaaaatctggatccccaggtaaggatagcagatggttttcagttatctccagtccacgttcacaaaatgtgaaggtgtggagacacttacaaagctgcctcacttctcactgtaaacattagctctttccactgcctacctggaccccagtctaggaattaaatctgcacctaaccaaggtcccttgtaagaaatgtccattcaagcagtcattctctgggtatataatatgattttgactaccttatctggtgttaagatttgaagttggccttttattggactaaaggggaactcctttaagggtctcagttagcccaagtttcttttgcttatatgttaatagttttaccctctgcattggagagaggagtgctttactccaagaagctttcctcatggttaccgttctctccatcatgccagccttctcaacctttgcagaaattactagagaggatttgaatgtgggacacaaaggtcccatttgcagttagaaaatttgtgtccacaaggacaagaacaaagtatgagctttaaaactccataggaaacttgttaatcaacaaagaagtgttaatgctgcaagtaatctcttttttaaaactttttgaagctacttattttcagccaaataggaatattagagagggactggtagtgagaatatcagctctgtttggatggtggaaggtctcattttattgagatttttaagatacatgcaaaggtttggaaatagaacctctaggcaccctcctcagtgtgggtgggctgagagttaaagacagtgtggctgcagtagcatagaggcgcctagaaattccacttgcaccgtagggcatgctgataccatcccaatagctgttgcccattgacctctagtggtgagtttctagaatactggtccattcatgagatattcaagattcaagagtattctcacttctgggttatcagcataaactggaatgtagtgtcagaggatactgtggcttgttttgtttatgtttttttttcttattcaagaaaaaagaccaaggaataacattctgtagttcctaaaaatactgacttttttcactactatacataaagggaaagttttattcttttatggaacacttcagctgtactcatgtattaaaataggaatgtgaatgctatatactctttttatatcaaaagtctcaagcacttatttttattctatgcattgtttgtcttttacataaataaaatgtttattagattg

SEQ ID NO: 7 - DNA, 353 bp, Homo sapiens
  misc_feature (1)..(353): n is a, c, g, or t
cggggctaagggatggaaagacttttnttgagagaacgantggntctaatggattaaatcttaaaagcttctnggngttttccacaaaggggtcttgtggattgttgagagagtcgatgctcactccaaatgatgtgctagtgatcacatccatgctgtaggccccaaagacgtntttcaaggtgacaggcttgccttctncctgcttcccgcctcagatttctcaccaacacatctccatactggggcaatgataggggaccatctccntgagttttccgctgggtgaatnttgggagacagcaatgatcggnattcccctncccattcttccccgggncccaattcttggg

SEQ ID NO: 8 - DNA, 2768 bp, Homo sapiens
cactgctgtgcagggcaggaaagctccatgcacatagcccagcaaagagcaacacagagctgaaaggaagactcagaggagagagataagtaaggaaagtagtgatggctctcatcccagacttggccatggaaacctggcttctcctggctgtcagcctggtgctcctctatctatatggaacccattcacatggactttttaagaagcttggaattccagggcccacacctctgccttttttgggaaatattttgtcctaccataagggcttttgtatgtttgacatggaatgtcataaaaagtatggaaaagtgtggggcttttatgatggtcaacagcctgtgctggctatcacagatcctgacatgatcaaaacagtgctagtgaaagaatgttattctgtcttcacaaaccggaggccttttggtccagtgggatttatgaaaagtgccatctctatagctgaggatgaagaatggaagagattacgatcattgctgtctccaaccttcaccagtggaaaactcaaggagatggtccctatcattgcccagtatggagatgtgttggtgagaaatctgaggcgggaagcagagacaggcaagcctgtcaccttgaaagacgtctttggggcctacagcatggatgtgatcactagcacatcatttggagtgaacatcgactctctcaacaatccacaagacccctttgtggaaaacaccaagaagcttttaagatttgattttttggatccattctttctctcaataacagtctttccattcctcatcccaattcttgaagtattaaatatctgtgtgtttccaagagaagttacaaattttttaagaaaatctgtaaaaaggatgaaagaaagtcgcctcgaagatacacaaaagcaccgagtggatttccttcagctgatgattgactctcagaattcaaaagaaactgagtcccacaaagctctgtccgatctggagctcgtggcccaatcaattatctttatttttgctggctatgaaaccacgagcagtgttctctccttcattatgtatgaactggccactcaccctgatgtccagcagaaactgcaggaggaaattgatgcagttttacccaataaggcaccacccacctatgatactgtgctacagatggagtatcttgacatggtggtgaatgaaacgctcagattattcccaattgctatgagacttgagagggtctgcaaaaaagatgttgagatcaatgggatgttcattcccaaaggggtggtggtgatgattccaagctatgctcttcaccgtgacccaaagtactggacagagcctgagaagttcctccctgaaagattcagcaagaagaacaaggacaacatagatccttacatatacacaccctttggaagtggacccagaaactgcattggcatgaggtttgctctcatgaacatgaaacttgctctaatcagagtccttcagaacttctccttcaaaccttgtaaagaaacacagatccccctgaaattaagcttaggaggacttcttcaaccagaaaaacccgttgttctaaaggttgagtcaagggatggcaccgtaagtggagcctgaattttcctaaggacttctgctttgctcttcaagaaatctgtgcctgagaacaccagagacctcaaattactttgtgaatagaactctgaaatgaagatgggcttcatccaatggactgcataaataaccggggattctgtacatgcattgagctctctcattgtctgtgtagagtgttatacttgggaatataaaggaggtgaccaaatcagtgtgaggaggtagatttggctcctctgcttctcacgggactatttccaccacccccagttagcaccattaactcctcctgagctctgataagagaatcaacatttctcaataatttcctccacaaattattaatgaaaataagaattattttgatggctctaacaatgacatttatatcacatgttttctctggagtattctataagttttatgttaaatcaataaagaccactttacaaaagtattatcagatgctttcctgcacattaaggagaaatctatagaactgaatgagaaccaacaagtaaatatttttggtcattgtaatcactgttggcgtggggcctttgtcagaactagaatttgattattaacataggtgaaagttaatccactgtgactttgcccattgtttagaaagaatattcatagtttaattatgccttttttgatcaggcacagtggctcacgcctgtaatcctagcagtttgggaggctgagccgggtggatcgcctgaggtcaggagttcaagacaagcctggcctacatggttgaaaccccatctctactaaaaatacacaaattagctaggcatggtggactcgcctgtaatctcactacacaggaggctgaggcaggagaatcacttgaacctgggaggcggatgttgaagtgagctgagattgcaccactgcactccagtctgggtgagagtgagactcagtcttaaaaaaatatgcctttttgaagcacgtacattttgtaacaaagaactgaagctcttattatattattagttttgatttaatgttttcagcccatctcctttcatatttctgggagacagaaaacatgtttccctacacctcttgcattccatcctcaacacccaactgtctcgatgcaatgaacacttaataaaaaacagtcgattggtc

SEQ ID NO: 9 - DNA, 255 bp, Homo sapiens
  misc_feature (1)..(255): n = a, c, g, or t
ttagaagaaataaaaatttgtctttatttaaaatgtggcaattgggaagtacagtacatgcttccctgtggcttgggggtgtgtagttggggccctaattcttgagccagtcaagagacacttgggatttagccctcaattgcagcactgaggcactggtgcgaagnaaacaatcctgagggggtccccaacccacctagtcactccttagtttgcagaggcactatctgcagagtcctgtgaaaggctgcagtg

SEQ ID NO: 10 - DNA, 1873 bp, Homo sapiens
cgcgccccaggccggtgtacccccgcactccgcgccccggcctagaagctctctctccccgctccccggcccggcccccgccccgccccgccccagcccgctgggccgccatggagcgctggccttggccgtcgggcggcgcctggctgctcgtggctgcccgcgcgctgctgcagctgctgcgctcagacctgcgtctgggccgcccgctgctggcggcgctggcgctgctggccgcgctcgactggctgtgccagcgcctgctgcccccgccggccgcactcgccgtgctggccgccgccggctggatcgcgttgtcccgcctggcgcgcccgcagcgcctgccggtggccactcgcgcggtgctcatcaccggctgtgactctggttttggcaaggagacggccaagaaactggactccatgggcttcacggtgctggccaccgtattggagttgaacagccccggtgccatcgagctgcgtacctgctgctcccctcgcctaaggctgctgcagatggacctgaccaaaccaggagacattagccgcttgctagagttcaccaaggcccacaccaccagcaccggcctgtggggcctcgtcaacaacgcaggccacaatgaagtagttgctgatgcggagctgtctccagtggccactttccgtagctgcatggaggtgaatttctttggcgcgctcgagctgaccaagggcctcctgcccctgctgcgcagctcaaggggccgcatcgtgactgtggggagcccagcgggggacatgccatatccgtgcttgggggcctatggaacctccaaagcggccgtggcgctactcatggacacattcagctgtgaactccttccctggggggtcaaggtcagcatcatccagcctggctgcttcaagacagagtcagtgagaaacgtgggtcagtgggaaaagcgcaagcaattgctgctggccaacctgcctcaagagctgctgcaggcctacggcaaggactacatcgagcacttgcatgggcagttcctgcactcgctacgcctggccatgtccgacctcaccccagttgtagatgccatcacagatgcgctgctggcagctcggccccgccgccgctattaccccggccagggcctggggctcatgtacttcatccactactacctgcctgaaggcctgcggcgccgcttcctgcaggccttcttcatcagtcactgtctgcctcgagcactgcagcctggccagcctggcactaccccaccacaggacgcagcccaggacccaaacctgagccccggcccttccccagcagtggctcggtgagccatgtgcacctatggcccagccactgcagcacaggaggctccgtgagccttggttcctccccgaaaacccccagcattacgatcccccaagtgtcctggaccctggcctaaagaatcccacccccacttcatgcccactgccgatgcccaatccaggcccggtgaggccaaggtttcccagtgagcctctgcgcctctccactgtttcatgagcccaaacaccctcctggcacaacgctctaccctgcagcttggagaactccgctggatgggagtctcatgcaagacttcactgcagcctttcacaggactctgcagatagtgcctctgcaaactaaggagtgactaggtgggttggggaccccctcaggattgtttctcggcaccagtgcctcagtgctgcaattgagggctaaatcccaagtgtctcttgactggctcaagaattagggccccaactacacacccccaagccacagggaagcatgtactgtacttcccaattgccacattttaaataaagacaaatttttatttcttcta

SEQ ID NO: 11 - DNA, 267 bp, Homo sapiens
  misc_feature (1)..(267): n = a, c, g, or t
tttttttttttttttttttttttggggattcaaatcctttattgacggtggtggtgaggccagccctccctggcaggctctgggtgcatctccctagtatgttctgctgggagccgggcaccggggcaggcgtaggggcagcgagtggcctaggactcggactcaaacatcttcttccggccctccatgccagacttctcctcgatgttcttcctccagtcacccacgtctcgcaggtnccgnnccttctctgtggtcctccttctt

SEQ ID NO: 12 - DNA, 701 bp, Homo sapiens
ctaggctccaagctcaggacctcaggatgggagatgaggagaagcggaacagggccatcacggcccgcaggcagcacctgaagagcgtgatgctgcagatagcggccacggagctggagaaggaggagagccgccgtgaggcagagaagcagaactacctggcggagcactgcccgccgctgcatatcccgggctccatgtctgaagtgcaggagctctgcaaacagctgcacgccaagatcgatgcggctgaagaggagaagtacgacatggaggtgagggtgcagaagaccagcaaggagctggaggacatgaaccagaagctatttgatctgcggggcaagttcaagcggcccccactgcggagggtgcgcatgtcggccgatgccatgctcaaggccctgctgggctcgaagcacaaggtgtgcatggacctgagggccaacctgaagcaggtcaagaaggaggacacagagaaggagcgggacctgcgagacgtgggtgactggaggaagaacatcgaggagaagtctggcatggagggccggaagaagatgtttgagtccgagtcctaggccactcgctgcccctacgcctgccccggtgcccggctcccagcagaacatactagggagatgcacccagagcctgccagggagggctggcctcaccaccaccgtcaataaaggatttgaatcccc

SEQ ID NO: 13 - DNA, 384 bp, Homo sapiens
cactgggccgggctgcctgtgtgctcacaggctgtttatttatccaagagtaaggaggagagagacaggatgaagagaaaaaccagactccccacggtggcagggctgaggggaaaaacaaacagaaaatctcctcttctttctcttgctgctgaccccagggtactccagcttcagatgggacctctgggggtggcttaggggcctcgaggagcgagcacagctcagctgttggctggggtcaggatccgcacaggggcaggtcccagactcggaatggcttctgtgtacacaaaatatctctctgcaaaaggcactggtggaggggggcagcaggaaggccaccccatcacaggcagagtccactctgtcctgcgtctgggt

SEQ ID NO: 14 - DNA, 2345 bp, Homo sapiens
cccttctgacccaccccgctaggtagtcccacacggcggccctcggctcccgggccccgccccggcctcctgcatttgcccgaggccccctccccagcactggccacaccccagggtagccccgcccccagagctcctcccccagcggccggggtagggtggcggctggcccagccgggcccccgctgccctctgccctgggcgcttggtcggagcgggctgggcaaagcgggacagggcgagcgcggcggcccgggggctcggaggcgaagatggcgtccggcaggcgcgccccgcgcaccgggctgctggagctgcgcgccggggcgggctcgggggccggcggcgagcgatggcagcgggtgctgctgagtctggcggaggacgtgctgaccgtgagccccgccgacggcgaccctggtcccgagcccggcgctccgcgggagcaggagcccgcgcagctcaacggcgccgcggagccgggcgccgggcccccgcagctgccagaggcgctactgctccagcggcgccgcgtgacggtgcgcaaggccgacgccggtgggctgggcatcagcatcaaaggcggccgggagaacaagatgcctattctcatttccaagatcttcaagggattggcagctgaccagacagaggccctttttgtgggggatgccatcctgtctgtgaatggggaagacttgtcctctgctacccatgatgaggcggtgcaggtcctcaagaagacaggcaaggaggtggtgctggaggtcaagtatatgaaggacgtctcaccgtatttcaagaactctactggtgggacctcggtcggctgggactcacctcctgcctcaccccttcagcggcagccttcctcccctggccccacaccccggaacttcagcgaggccaaacacatgtccttgaagatggcatatgtctcgaagaggtgcacccccaatgacccggagcccaggtatctggagatctgctcggcagatggtcaagacaccctcttcctgagggccaaggatgaggctagtgcgaggtcgtgggcgactgccatccaagcccaggtcaatactctgacgccgcgggtcaaggatgagctgcaggcactgttggcagccaccagcacagctgggagccaggacatcaagcagattggctggctaactgagcagctgcccagtgggggcacagcccccaccctggccctgctaactgaaaaggaactgctcctctacttgtctctccccgagacccgcgaggccctgagccggccagcccgtactgccccactcatcgccaccagactggtgcactcaggcccctccaagggctcagtgccctacgatgcagagctctcttttgccctgcgcacgggcacgcgtcacggtgtggacactcacctgttcagcgtggagtcaccgcaggagctggctgcctggacccgccagcttgtggatggctgtcaccgggccgccgagggtgtgcaggaggtgtctacagcctgcacgtggaatgggcgtccctgcagcctgtctgtgcacatcgacaagggcttcacactgtgggcggctgagccaggtgcagcccgagctgtgctcctgcgacagcccttcgagaagctgcagatgtcttcagatgacggtgccagtctccttttcctggattttggaggtgctgaaggcgagatccagctggacctgcactcgtgtcccaaaaccatagtcttcatcatccactccttcctgtcggccaaagtcacccgcctcgggctgttggcctagaagtcgccggatgcactagccctgaagaggggtgtccatgacatggcctgagctgggcctccaccgactgcctgctcacccctgggctgagggaagggagaggagaggaacaagggcctccgaaaccccaaccctgagggagactggattggtcttggggcccaggacccagacgcaggacagagtggactctgcctgtgatggggtggccttcctgctgcccccctccaccagtgccttttgcagagagatattttgtgtacacagaagccattccgagtctgggacctgcccctgtgcggatcctgaccccagccaacagctgagctgccgggcctcctcgaggcccctaagccacccccagaggtcccatctgaagctggagtaccctggggtcagcagcaagagaaagaagaggagattttctgtttgtttttcccctcagccctgccaccgtggggagtctggtttttctcttcatcctgtctctctcctccttactcttggataaataaacagcctgtgagcacacaggcagcccggcccagtgaaaaa

SEQ ID NO: 15 - DNA, 353 bp, Homo sapiens
  misc_feature (1)..(353): n is a, c, g, or t
ttttgtggtggctttttattactcggtgtggagtgggtgtgggggcaggcaggagtggtggctcccactaggccagctccccatttccaaacaggagctgcctggggtgcccaggaggncccgggaactgggggagtgcaggccgganggttgcgagcgaggagcagatctttggtgaaggaggccaggctctatttccagcgcccggtgactttagccttcccgcgggtcttggagactttctggttatcgttgatcctgtttcggagaacattgatctcatatttctgctgcttgaacttctcctgcaggtcgaacttctctgcctccaagttatagatgctctgccacat

SEQ ID NO: 16 - DNA, 1123 bp, Homo sapiens
cgggaaacctgcactgacttttttctccttttggagggagagcagagaccatgtctgacatagaagaggtggtggaagagtacgaggaggaggagcaggaagaagcagctgttgaagagcaggaggaggcagcggaagaggatgctgaagcagaggctgagaccgaggagaccagggcagaagaagatgaagaagaagaggaagcaaaggaggctgaagatggcccaatggaggagtccaaaccaaagcccaggtcgttcatgcccaacttggtgcctcccaagatccccgatggagagagagtggactttgatgacatccaccggaagcgcatggagaaggacctgaatgagttgcaggcgctgattgaggctcactttgagaacaggaagaaagaggaggaggagctcgtttctctcaaagacaggatcgagagacgtcgggcagagcgggccgagcagcagcgcatccggaatgagcgggagaaggagcggcagaaccgcctggctgaagagagggctcgacgagaggaggaggagaacaggaggaaggctgaggatgaggcccggaagaagaaggctttgtccaacatgatgcattttgggggttacatccagaagcaggcccagacagagcggaaaagtgggaagaggcagactgagcgggaaaagaagaagaagattctggctgagaggaggaaggtgctggccattgaccacctgaatgaagatcagctgagggagaaggccaaggagctgtggcagagcatctataacttggaggcagagaagttcgacctgcaggagaagttcaagcagcagaaatatgagatcaatgttctccgaaacaggatcaacgataaccagaaagtctccaagacccgcgggaaggctaaagtcaccgggcgctggaaatagagcctggcctccttcaccaaagatctgctcctcgctcgcacctgcctccggcctgcactcccccagttcccgggccctcctgggcaccccaggcagctcctgtttggaaatggggagctggcctaggtgggagccaccactcctgcctgcccccacacccactccacaccagtaataaaaagccaccacacactgaaaaaaaaaaa

SEQ ID NO: 17 - DNA, 467 bp, Homo sapiens
tttttttttttttttttttttgctagtcataattcttgttttattattattctgaaaataatagtttatggaggaaactcctgattaaagccatgtcaggagcccgtccgaggatacaacagttgatatacctccttttaatttcacacagggtcttgctttgtcacccaggctgcagtgcagtgctgtgaatcatggctcactgcagccttgacctcctaggctcaaatgatcctcccacctcagcctcctgagtagctgggaccacagggacatgccgccacatctggctaatttttgcattttttgtagagacgaggtctcaccatgttgtccaggctggtcctagactcctgggctcaagtgatccccttgccttggcctcccacagtgttaagattacaggtgtgagccatggcacccaacgatatcccttctttttattggcaagaaagatctttcccctt

SEQ ID NO: 18 - DNA, 3759 bp, Homo sapiens
ccttttgtgggagtccggtctgtccacttgccggtccctcagaccgtcggcggtctctgtccgcttcgggacctgtccgctggtcgctccgcgtccgatggctcctggccgcggaaccttaggcctggccctggtctccgagcgcgggttcgccgggaggagcgtgtggcgggggtgtgccggggcgtgagtgcgccgagcatggggctgagcctggtgtggggagtgggtatctgcggagccggcctgaaccccacctcagccgggcgcggggagggggctccgtgcgtgtgatcgtgcagctgtgagcgcgtggccgccccgcggggctccgctgcaggcccctcagccccaggagcagtactcgctcttcagggcctgccctggatcctggaggctacacagctgcccactcctcctggggaggctgccgtggaggccatggagatccctgccccggagcccgagaagacaggtacagcttcactcttgtagtcagtatgtctgtggatttgcacttgaggatattgacactcagagagatcaggccagtttcccagaagcatccagcatcttttttttttttttttaaatggaatctcactctgtcacccaggcgggagtggtgcaatggcttgatcttccgctcactgcaacctctgcttcccgggttcaaactattctcccgcctcaacctcccaagtagttgggattgcaggcacgcgccaccacgcccggctaatgtttgtatttttagtagagacggggttttgccatgtttcccaggctggtcttaagctcaagcgatccgcccgcctcggcctcccaaagtgctgggattacaggcgtgagccgatcggctcctggcctcctgcttctttcttccccgcgcccaatcatttgttcccactcagacgtgcttgattggcggtagcagctcttttcgtcatgaattggagtgtctgagacacaggcagattattcctcgatgctgtttctgttggtgctagctactggggctcgttccccacctggccttgctggcaggttctgcctcctctctgattctcacttgtgccacgacaggtgggctttaagctcagccgtgagcctggtagccgtgaccttgactcatttctccttcgccagctctttcctctcaggatcctgctctttccctgaaagagaatctcgaggatatatcgggttggggtcttcccgaagccaggtccaaggaatcggtgagtttcaaggatgtggctgtggacttcacccaggaggagtggggtcaactagactcccctcagagggccttgtaccgggatgtgatgttggagaactaccagaaccttcttgccctaggacctccactgcacaagccagatgtgatctctcatctggaacgaggcgaggagccatggagcatgcagagggaagtccccagagggccctgtccagaatgggagctgaaggcggtgccctctcaacagcagggcatttgcaaagaagaaccggcccaggagcccatcatggagcggcccctcggcggggcgcaggcgtgggggcgccaggcaggtgctctgcagaggagtcaggctgcgcctgggcgccgcacctgccatggtctgggacgtcctgtagaggaattccccctcaggtgtcccctcttcgcccagcaacgcgttcccgaggggggacccttgctggacacacgcaagaacgtccaggccactgagggcagaaccaaggcccccgcgagactgtgtgcaggggaaaacgcctccacgccaagtgagccagaaaagttcccccaggtgcgccggcagcgcggggcgggcgccggggagggcgagttcgtgtgcggcgagtgcgggaaggcgttccgccagagctcctccctcacgctgcaccggcgctggcacagccgggagaaggcttacaagtgcgatgaatgcggcaaggccttcacctggagcaccaaccttctggagcaccggcgcatccacaccggcgagaagcccttcttctgcggcgagtgcgggaaggccttcagctgccactcgtccctcaacgtgcaccagcgcatccacacgggcgagcggccctacaagtgcagcgcctgcgagaaggccttcagctgcagctcgctgctcagcatgcacctgcgggtgcacaccggcgagaagccctaccggtgcggcgagtgcggcaaggccttcaaccagcgtacacacctcacacgccaccaccgcatccacacgggcgagaagccctaccagtgcggctcctgcggcaaggccttcacctgccactcatccctcaccgtgcatgagaagatccacagcggggacaagccgttcaagtgcagcgactgcgagaaggccttcaacagccgctcgcgcctcaccctccaccagaggacgcacacgggcgagaagcccttcaagtgcgccgactgcgggaagggcttcagctgccacgcgtacctgctcgtgcaccggcgcatccacagcggcgagaagcccttcaagtgcaacgagtgcggcaaagccttcagctcccacgcctacctcatcgtgcaccggcgcatccacacaggcgagaagcccttcgactgcagccagtgttggaaggccttcagctgccactcgtccctcatcatgcaccagcgcatccacaccggtgagaagccctacaagtgcagcgagtgcggcagagccttcagccagaaccactgtctcattaaacatcagaaaatccactccggggagaagtcgtttaagtgtgagaaatgtggggagatgttcaactggagctcgcacctcactgagcaccagaggctgcacagcgaggggaagcccttggccatccagttcaacaaacacctgctcagcacatactacgtgcctggcagcctgctgggtgcaggggatgctggactgagggacgtggatcccatcgacgcgctggatgtggcaaagctcttgtgcgtggttccccccagagctggcaggaatttctccctggggagcaaacctcgaaactaacatgatgtgctttggtgtcagtagctgctttctgagctactcaacaaggaaagcaccctggtcctccctggctcctagatccagaccaccttcctccaggtgtgggagccttgccttatcaccccaatcaggtctgcatgccagggtgcctcctctagttaaagtcagtcacctccccagaagggccacactccaggaggagtgttgagagtcatttgaggtagtcttgccacctgttttccttgatgggcctggaagttgttgacaaggggaaagatctttcttgccaataaaaagaagggatatcgttgggtgccatggctcacacctgtaatcttaacactgtgggaggccaaggcaaggggatcacttgagcccaggagtctaggaccagcctggacaacatggtgagacctcgtctctacaaaaaatgcaaaaattagccagatgtggcggcatgtccctgtggtcccagctactcaggaggctgaggtgggaggatcatttgagcctaggaggtcaaggctgcagtgagccatgattcacagcactgcactgcagcctgggtgacaaagcaagaccctgtgtgaaattaaaaggaggtatatcaactgttgtatcctcggacgggctcctgacatggctttaatcaggagtttcctccataaactattattttcagaataataataaaacaagaattatgactagc

SEQ ID NO: 19 - DNA, 567 bp, Homo sapiens
  misc_feature (1)..(567): n is a, c, g, or t
agtccaagatggcagtcggaactctgacccataaaaccagaaggctttagatgaagaacaaaaagaagggaaaccaaccctgcttccttttgcctgttattaatcgctgcaataaagccattagaatctaccaacatatcacccaacttgaaaagaagagagattctgaaaaatagtcagaaaagaagggtaaatggaaatgatgagtccctcccctttatggctattttaagaagcacaccatgttattcatatggcccgtgcttagcccactccctgaattgtttgattttagtggatataaaaattcttcctaagggctttcttagttccactgccagcagtagttccccatttctatttctcgttaggtctcaactcttccgtcatgagttctatgttccccaggattcnaagagcccaaatcctgcggtgggagtgaaggggaaggcagatgctggctactggattggctctgggactcagagtgggagtcactgtggaaatcactattnccaagttctttggcagtgggagggtggtggggggangggggtgatggaagag

SEQ ID NO: 20 - DNA, 2156 bp, Homo sapiens
ggcacgagcccagaaacaaagacttcacggacaaagtcccttggaaccagagagaagccgggatggaaactccaaacaccacagaggactatgacacgaccacagagtttgactatggggatgcaactccgtgccagaaggtgaacgagagggcctttggggcccaactgctgccccctctgtactccttggtatttgtcattggcctggttggaaacatcctggtggtcctggtccttgtgcaatacaagaggctaaaaaacatgaccagcatctacctcctgaacctggccatttctgacctgctcttcctgttcacgcttcccttctggatcgactacaagttgaaggatgactgggtttttggtgatgccatgtgtaagatcctctctgggttttattacacaggcttgtacagcgagatctttttcatcatcctgctgacgattgacaggtacctggccatcgtccacgccgtgtttgccttgcgggcacggaccgtcacttttggtgtcatcaccagcatcatcatttgggccctggccatcttggcttccatgccaggcttatacttttccaagacccaatgggaattcactcaccacacctgcagccttcactttcctcacgaaagcctacgagagtggaagctgtttcaggctctgaaactgaacctctttgggctggtattgcctttgttggtcatgatcatctgctacacagggattataaagattctgctaagacgaccaaatgagaagaaatccaaagctgtccgtttgatttttgtcatcatgatcatcttttttctcttttggaccccctacaatttgactatacttatttctgttttccaagacttcctgttcacccatgagtgtgagcagagcagacatttggacctggctgtgcaagtgacggaggtgatcgcctacacgcactgctgtgtcaacccagtgatctacgccttcgttggtgagaggttccggaagtacctgcggcagttgttccacaggcgtgtggctgtgcacctggttaaatggctccccttcctctccgtggacaggctggagagggtcagctccacatctccctccacaggggagcatgaactctctgctgggttctgactcagaccataggaggccaacccaaaataagcaggcgtgacctgccaggcacactgagccagcagcctggctctcccagccaggttctgactcttggcacagcatggagtcacagccacttgggatagagagggaatgtaatggtggcctggggcttctgaggcttctggggcttcagtcttttccatgaacttctcccctggtagaaagaagatgaatgagcaaaaccaaatattccagagactgggactaagtgtaccagagaagggcttggactcaagcaagatttcagatttgtgaccattagcatttgtcaacaaagtcacccacttcccactattgcttgcacaaaccaattaaacccagtagtggtgactgtgggctccattcaaagtgagctcctaagccatgggagacactgatgtatgaggaatttctgttcttccatcacctccccccccccgccaccctcccactgccaagaacttggaaatagtgatttccacagtgactccactctgagtcccagagccaatcagtagccagcatctgcctccccttcactcccaccgcaggatttgggctcttggaatcctggggaacatagaactcatgacggaagagttgagacctaacgagaaatagaaatgggggaactactgctggcagtggaactaagaaagcccttaggaagaatttttatatccactaaaatcaaacaattcagggagtgggctaagcacgggccatatgaataacatggtgtgcttcttaaaatagccataaaggggagggactcatcatttccatttacccttcttttctgactatttttcagaatctctcttcttttcaagttgggtgatatgttggtagattctaatggctttattgcagcgattaataacaggcaaaaggaagcagggttggtttcccttctttttgttcttcatctaagccttctggttttatgggtcagagttccgactgccatcttggacttgtcagcaaaaaaaaaaaaaaaaa

SEQ ID NO: 21 - DNA, 446 bp, Homo sapiens
ggcagacacttccatttaatgactaaaaatcacacatctcaggtcacgggtctaggagaaaacacacacacacacacacacacacacacacacacacacggattccccatcaaggggacatttgcagtttccaaaccttgaagatactgaagggaccagaaagttcctttgagtggctggtcacccaaagctcccggtcctccacccactgccctttggagggactcaaaccttgggaggagaaggctgagcttcctgtgggcccctcccacccacacctgagccagagagaagactgcagcaaagacatccaaagccaacgcaatgggaagcgtccgagatggcagaggagccagccctgtccttggctcacccagcttccaccatacaggaacccaagaccccagccttgcttccacagagaactggcaggggtcccctggcct

SEQ ID NO: 22 - DNA, 1846 bp, Homo sapiens
tgatcacacccacagttgagctgcgctggccagagatgcctgcccacagcctggtgatgagcagcccggccctcccggccttcctgctctgcagcacgctgctggtcatcaagatgtacgtggtggccatcatcacgggccaagtgaggctgcggaagaaggcctttgccaaccccgaggatgccctgagacacggaggcccccagtattgcaggagcgaccccgacgtggaacgctgcctcagggcccaccggaacgacatggagaccatctaccccttccttttcctgggcttcgtctactcctttctgggtcctaacccttttgtcgcctggatgcacttcctggtcttcctcgtgggccgtgtggcacacaccgtggcctacctggggaagctgcgggcacccatccgctccgtgacctacaccctggcccagctcccctgcgcctccatggctctgcagatcctctgggaagcggcccgccacctgtgaccagcagctgatgcctccttggccaccagaccatgggccaagagccgccgtggctatacctggggacttgatgttccttccagattgtggtgggccctgagtcctggtttcctggcagcctgctgcgcgtgtgggtctctgggcacagtgggcctgtgtgtgtgcccgtgtgtgtgtatgtgtatgtgtgtgtgtatgtttcttagccccttggattcctgcacgaagtggctgatgggaaccatttcaagacagattgtgaagattgatagaaaatccttcagctaaagtaacagagcatcaaaaacatcactccctctccctccctaacagtgaaaagagagaagggagactctatttaagattcccaaacctaatgatcatctgaatcccgggctaagaatgcagacttttcagactgaccccagaaattctggcccagccaatctagaggcaagcctggccatctgtatttttttttttccaagacagagtcttgctctgttgcccaagctggagtgaagtggtacaatctggctcactgcagcctccgcctcccgggttcaagcgattctcccgcctcagcctcctgagtagctgggattacaggcgcgtatcaccatacccagctaatttttgtatttttagtagagacgggttcaccatgttgcccaggagggtctcgaactcctggcctcaagtgatccaccggcctcggcctcccaaagtgctgggatgacaggcatgaatcactgtgctcagccaccatctggagttttaaaaggctcccatgtgagtccctgtgatggccaggccaggggacccctgccagttctctgtggaagcaaggctggggtcttgggttcctgtatggtggaagctgggtgagccaaggacagggctggctcctctgcccccgctgacgcttcccttgccgttggctttggatgtctttgctgcagtcttctctctggctcaggtgtgggtgggaggggcccacaggaagctcagccttctcctcccaaggtttgagtccctccaaagggcagtgggtggaggaccgggagctttgggtgaccagccactcaaaggaactttctggtcccttcagtatcttcaaggtttggaaactgcaaatgtccccttgatggggaatccgtgtgtgtgtgtgtgtgtgtgtgtgtgtgtgtgtgtgtgtgtgttttctcctagacccgtgacctgagatgtgtgatttttagtcattaaatggaagtgtctgccaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 23 - DNA, 522 bp, Homo sapiens
gcggccgcgtagccacatcgccgtttcgagtacaaatacagcttcaaggggccgcacctggtgcagagcgacgggaccgtgcccttctgggcccacgcggggaatgctattccaagttcagatcaaattcgagtagcaccatctttaaaaagccaaagaggctcagtgtggacaaagacaaaagcggcctttgagaactgggaagttgaggtgacatttcgagtgactggaagaggtcggattggagctgatggcctagcaatttggtatgcagaaaatcaaggcttggagggccctgtgtttggatcagctgatctgtggaatggtgttggaatattttttgattcttttgacaatgatggaaagaaaaataatcctgctatagtaattataggcaacaatggacaaatccattatgaccatcagaatgacggggctagtcaagctttggcaagcttgcagagggacttccggcagaaaccctatcctgtccgagccagattacctattaccgaacacact

SEQ ID NO: 24 - DNA, 2764 bp, Homo sapiens
ctctaaagcttagagccaagatggcgggatccaggcaaaggggtctccgggccagagttcggccgctgttctgcgccttgctgctgtcactcggtcgcttcgtccggggcgacggcgtgggaggagaccccgcggtcgcgttgccacatcgccgtttcgagtacaaatacagcttcaaggggccgcacctggtgcagagcgacgggaccgtgcccttctgggcccacgcggggaatgctattccaagttcagatcaaattcgagtagcaccatctttaaaaagccaaagaggctcagtgtggacaaagacaaaagcggcctttgagaactgggaagttgaggtgacatttcgagtgactggaagaggtcgaattggagctgatggcctagcaatttggtatgcagaaaatcaaggcttggagggccctgtgtttggatcagctgatctgtggaatggtgttggaatattttttgattcttttgacaatgatggaaagaaaaataatcctgctatagtaattataggcaacaatggacaaatccattatgaccatcaaaatgacggggctagtcaagctttggcaagttgccagagggacttccgcaacaaaccctatcctgtccgagcaaagattacctattaccagaacacactgacagtaatgatcaataatggctttacaccagataaaaatgattatgaattttgtgccaaagtggaaaatatgattatccctgcacaagggcattttggaatatctgctgcaactggaggtcttgcagatgaccatgatgtcctttcttttctgactttccagttgactgaacctggaaaagagccgcccacaccagataaagaaatttcggaaaaggaaaaagaaaagtatcaggaggaatttgagcactttcaacaagaattggataaaaaaaaagaggaattccagaagggccaccccgacctccaagggcagcctgcggaggaaatatttgagagtgtaggagatcgagagctaagacaagtctttgaaggacagaatcgtattcatcttgaaatcaagcagctgaaccggcagttagatatgattcttgatgaacagagaagatatgtctcttccttaacagaggaaatctctaaaagaggagcaggaatgcctgggcagcatgggcagattactcaacaagaactggatactgttgtgaaaactcagcatgagattctgagacaagtaaatgaaatgaaaaattccatgagtgaaaccgtcagactggtcagtggaatgcagcaccctggctctgctggaggcgtctatgagacaacacagcacttcattgacatcaaagagcacctgcacatagtaaagagggacatagataacttagtgcagcgaaatatgccatcaaatgaaaagccgaaatgcccagaactaccaccatttccatcatgtttgtctacggtccacttcattatatttgttgtggtgcaaactgtattattcattggttatatcatgtataggtctcagcaagaagcagctgccaaaaaattcttttgactaccattttcctgtgtacttcatctatttgtgtacaaaatgatgtcgttttgagggaatttaagtatttaaattgcttcatagtctaaattattaattttcttaataaaataactgtttaaacattgatttgcagttaagaataaaccttaaagcaaagacaaccacattttaatttgttcacagtatgtaaatctgtctaaatttcagtgaatttctggtcagtatgatgcagcctctgagcagatattgaccagtaagagggtaaataaagtgggggcaacccctggatatgaatgttaccccctaagtctccaatattgcaggtttccctgtataacgtaaacacacttgccctcatgcctcccagaatatgaggtctaattaagaagtcccatcaggtttattttgtaaccaaagtcttttttagaggtcagacttcctaatcaaaggcctgggcctgcagtcctttcatcttaatgcaacttcctttgaaatcaaagaatattttgtctgagagctttaaggatctggtaatagacttcaaaatgttaagtgaaattttttttcctctatttatcaatgatatatttcacttttaaaggaaattttggaggaaaatatagctgctttttgcctaaaaaaccttgtgggtggaaatattcctctgagaatggcttttataggtattttgcctggtaatgtattcattcatgattgcccatattcttgaatgtttcttcattccaatggggtcaggtcaatattatgaaaataatttttatatttatatttgtaactaagaatttatttctccctttactacacgatgtaaattcacgtcaaattcgatgatctgaggatttaaattcacaaaacctgccactacattctggtttacattagttacttcatgctggctggggttagtgaccatttgcatactcttttaaatcaaggaggctgtagtagaggcagttttaagattcttgaaggcaaaatttgaaaaacagtgaatacttctaattgtttccttttagtgccagaactaagacattgtgaagcacttgttagtaaacttaaccttgaaatgtcagactggaaggagtttttatgtctttgtgcatacttctgggtattacagaaacagtctgtaaataacattttaagatgcaaatttaattctgttcacagctgatttatactgattt

SEQ ID NO: 25 - DNA, 545 bp, Homo sapiens
acaaagacaattatttaagacctttattaacaggtgcttgcactttcttgacttttttgaaaaaatcaagttgtaaacttttattacaaattaaaaatgaagttcttaaaaatctcaacttgaccagatatgaaacaatttaaaaacctttaaaggcgtattgagaaaaaccaggcttttttaaaaaacacgtttgttattaccaaaaagagacgtctttaggtaaaaataataaaaaccccatgctgcatagataatgcagatagttctatttatctggtcaacgggcaaaaagcaagcactttaggtcttcagctccaatcttttgttcatttcttattgctggaatttcatatttcctcttgttggatgactaaaccggatgatggtagagatggtaaagcggcatttactcagccccgccctgctcagcctcgggagcggacgaattctcagctggtggatcggctgctttgtctcttgccatctgtggttaaggtttctgggcgtctgcgtcggtaatgaattgcggcgtaccacttgag

SEQ ID NO: 26 - DNA, 1474 bp, Homo sapiens
aaaaagaaatcaagaatgcaattttatttacaatagtcacgccggaaatacctagaaataaatttaactgaggatgtaaaagacctctacaaggagagttcaatgcgtagcgggagcggagagctgaccccagagagccctgggcagccccacctccgccgccggcctagttaccatcacaccccggagagcccgcagctgccgcagccggccccagtcaccatcaccgcaaccatgagcagcgaggccgagacccagcagccgcccgccgccccccccgccgcccccgccctcagcgccgccgacaccaagcccggcactaccggagcggcgcagggagcggtggcccgggcggctcacatcggcggcgctggcgcgggcgacaagaaggtcatcgcaacgaaggttttgggaacagtaaaatggttcaatgtaaggaacggatatggtttcatcaacaggaatgacaccaaggaagatgtatttgtacaccagactgccataaagaagaataaccccaggaagtaccttcgcagtgtaggagatggagagactgtggagtttgatgttgttgaaggagaaaagggtgcggaggcagcaaatgttacaggtcctggtggtgttccagttcaaggcagtaaatatgcagcagaccgtaaccattatagacgctatccacgtcgtaggggtcctccacgcaattaccagcaaaattaccagaatagtgagagtggggaaaagaacgagggatcggagagtgctcccgaagccaggcccaacaacgccggccctacgcaggcgaaggttcccaccttactacatgcggagacctatgggcgtcgaccacagtattccaaccctcctgtgcagggagaagtgatggagggtgctgacaaccagggtgcaggagaacaaggtagaccagtgaggcagatatgtatcggggatatagaccacgattccgcaggggccctcctcgccaaaagacagcctagagaggacggcaatgaagaagataaagaaaatcaaggagatgagacccaaggtcagcagccacctcaagctcggtaccgccgcaacttcaattaccgacgcagacgcccagaaaaccctaaaccacaagatggcaaagagacaaaagcagccgatccaccagctgagaattcgtccgctcccgaggctgagcagggcggggctgagtaaatgccggcttaccatctctaccatcatccggtttagtcatccaacaagaagaaatatgaaattccagcaataagaaatgaacaaaagattggagctgaagacctaaagtgcttgctttttgcccgttgaccagataaatagaactatctgcattatctatgcagcatggggtttttattatgttttacctaaagacgtctctttttggtaataacaaaccgtgttttttaaaaaagcctggtttttctcaatacgcctttaaaggaattcc

SEQ ID NO: 27 - DNA, 638 bp, Homo sapiens
  misc_feature (1)..(638): n is a, c, g, or t
tttgagaaaagccggcataaagcacttttattgcaataataaaacttgagactcataaatggtgctgggggaagggtgcagcaacgatttctcaccaaatcactacacaggacagcaaaggggtgagaaggggctgagggaggaaaagccaggaaactgagatcagcagagggagccaagcatcaaaaaacaggagatgctgaagctgcgatgaccagcatcattttcttaagagaacattcaaggatttgtcatgatggctgggctttcactgggtgttaagtctacaaacagcaccttcaattgaaactgtcaattaaagttcttaagatttaggaagtggtggagcttggaaagttatgagattacaaaattcctgaaagtccattagaaaaaccacaggatgaaaaaaaaaaagggcccaaagaaggcttcagaggtcccagctggcccagggacagatacctgtagtgtccagcattgcagttgncatgattctactttcaaaggcagagagtttaatgggagggtgggtgaggatagatggaggcaaaggctttccacatcccaacccagttttaggaagggctttaattaccccagagattccccacttggggagaatcagcccacaaagc

SEQ ID NO: 28 - DNA, 1578 bp, Homo sapiens
gcgacgcgccgaggtactaggcagagccgtggaaccgccgccaggtcgctgttggtccacgccgcccgtcgcgccgcccgcccgctcagcgtccgccgccgccatgggagtgcaggtggaaaccatctccccaggagacgggcgcaccttccccaagcgcggccagacctgcgtggtgcactacaccgggatgcttgaagatggaaagaaatttgattcctcccgggacagaaacaagccctttaagtttatgctaggcaagcaggaggtgatccgaggctgggaagaaggggttgcccagatgagtgtgggtcagagagccaaactgactatatctccagattatgcctatggtgccactgggcacccaggcatcatcccaccacatgccactctcgtcttcgatgtggagcttctaaaactggaatgacaggaatggcctcctcccttagctccctgttcttggatctgccatggagggatctggtgcctccagacatgtgcacatgagtccatatggagcttttcctgatgttccactccactttgtatagacatctgccctgactgaatgtgttctgtcactcagctttgcttccgacacctctgtttcctcttcccctttctcctcgtatgtgtgtttacctaaactatatgccataaacctcaagttattcattttattttgttttcattttggggtgaagattcagtttcagtcttttggatataggtttccaattaagtacatggtcaagtattaacagcacaagtggtaggttaacattagaataggaattggtgttggggggggggtttgcaagaatattttattttaattttttggatgaaatttttatctattatatattaaacattcttgctgctgcgctgcaaagccatagcagatttgaggcgctgttgaggactgaattactctccaagttgagagatgtctttgggttaaattaaaagccctacctaaaactgaggtggggatggggagagcctttgcctccaccattcccacccaccctccccttaaaccctctgcctttgaaagtagatcatgttcactgcaatgctggacactacaggtatctgtccctgggccagcagggacctctgaagccttctttgtggccttttttttttttcatcctgtggtttttctaatggactttcaggaattttgtaatctcataactttccaagctccaccacttcctaaatcttaagaactttaattgacagtttcaattgaaggtgctgtttgtagacttaacacccagtgaaagcccagccatcatgacaaatccttgaatgttctcttaagaaaatgatgctggtcatcgcagcttcagcatctcctgttttttgatgcttggctccctctgctgatctcagtttcctggcttttcctccctcagccccttctcacccctttgctgtcctgtgtagtgatttggtgagaaatcgttgctgcacccttcccccagcaccatttatgagtctcaagttttattattgcaataaaagtgctttatgccggcttttctcaaaaaaaaaaaaaaaaaa

SEQ ID NO: 29 - DNA, 625 bp, Homo sapiens
tgcttattaaactagttttactttagcaccattcttagtggagcaggattcttgatcatggggtggaattttgtgtatctgggcttcatgggatgcataaaattttccagttggtaagtagcaggtgccgagggtctggatcagaaaaaaaggcaggcagccgggttcccactcgccttccattgacttctcggcagctcaccggccatcatttgaagcaaacactcctgagggagctgtttgaggaagggcagacatccggcttcttgccctggtacaactggcagacaagggtaaggagctgggtctgatcttccaagggtttcccattgacggtgacccagggcacatactcgtgtggtggctggagagcatctgtccgctgggcgttggcgtgcatgagctgcatgccgcggtcccccattggccactccatgatagtgtctggcgacaccctggggcgttagagctgcaggcatagtggcagacttctctccatgtgctcaaactcttccatgcagacaatggtcagggaggctagctccatgtcaagttcatccaacacgcagggctcccacttggttgaatttgcactcttcttctccatggtggaaattggactccc

SEQ ID NO: 30 - DNA, 1051 bp, Homo sapiens
ggaccgccgcctggttaaaggcgcttatttcccaggcagccgctgcagtcgccacacctttgcccctgctgcgatgaccctgtcgccacttctgctgttcctgccaccgctgctgctgctgctggacgtccccacggcggcggtgcaggcgtcccctctgcaagcgttagacttctttgggaatgggccaccagttaactacaagacaggcaatctatacctgcgggggcccctgaagaagtccaatgcaccgcttgtcaatgtgaccctctactatgaagcactgtgcggtggctgccgagccttcctgatccgggagctcttcccaacatggctgttggtcatggagatcctcaatgtcacgctggtgccctacggaaacgcacaggaacaaaatgtcagtggcaggtgggagttcaagtgccagcatggagaagaggagtgcaaattcaacaaggtggaggcctgcgtgttggatgaacttgacatggagctagccttcctgaccattgtctgcatggaagagtttgaggacatggagagaagtctgccactatgcctgcagctctacgccccagggctgtcgccagacactatcatggagtgtgcaatgggggaccgcggcatgcagctcatgcacgccaacgcccagcggacagatgctctccagccaccacacgagtatgtgccctgggtcaccgtcaatgggaaacccttggaagatcagacccagctccttacccttgtctgccagttgtaccagggcaagaagccggatgtctgcccttcctcaaccagctccctcaggagtgtttgcttcaagtgatggccggtgagctgcggagagctcatggaaggcgagtgggaacccggctgcctgcctttttttctgatccagaccctcggcacctgctacttaccaactggaaaattttatgcatcccatgaagcccagatacacaaaattccaccccatgatcaagaatcctgctccactaagaatggtgctaaagtaaaactagtttaataagcaaaaaaaaaaaaaaaaaaa

SEQ ID NO: 31 - DNA, 369 bp, Homo sapiens
gggaatactgaagtatgtagtggtcttagggtattctcgggaaggtgacagtgaggggttcttcaaaggagaacagaggataaaaggctcaatgaaaggataatctccatattagtgctaccaaagtgtcattaatttctatttgttggaaactttactaaggaatgactgctttgaggtaatggataaggacagagcttgaagggtcagcaattcagtcagccactggagtagttttcacatgaagtgagaagaaaagctgagatggagtttgtagggcagctggagttcagatctctcctaagtccctcttctggttcagatattttggtcacctgtgtacagtatgttcacagggggtcccctggg

SEQ ID NO: 32 - DNA, 1157 bp, Homo sapiens
gctcacagtcatcaattatagaccccacaacatgcgccctgaagacagaatgttccatatcagagctgtgatcttgagagccctctccttggctttcctgctgagtctccgaggagctggggccatcaaggcggaccatgtgtcaacttatgccgcgtttgtacagacgcatagaccaacaggggagtttatgtttgaatttgatgaagatgagatgttctatgtggatctggacaagaaggagaccgtctggcatctggaggagtttggccaagccttttcctttgaggctcagggcgggctggctaacattgctatattgaacaacaacttgaataccttgatccagcgttccaaccacactcaggccaccaacgatccccctgaggtgaccgtgtttcccaaggagcctgtggagctgggccagcccaacaccctcatctgccacattgacaagttcttcccaccagtgctcaacgtcacgtggctgtgcaacggggagctggtcactgagggtgtcgctgagagcctcttcctgcccagaacagattacagcttccacaagttccattacctgacctttgtgccctcagcagaggacttctatgactgcagggtggagcactggggcttggaccagccgctcctcaagcactgggaggcccaagagccaatccagatgcctgagacaacggagactgtgctctgtgccctgggcctggtgctgggcctagtcggcatcatcgtgggcaccgtcctcatcataaagtctctgcgttctggccatgacccccgggcccaggggaccctgtgaaatactgtaaaggtgacaaaatatctgaacagaagaggacttaggagagatctgaactccagctgccctacaaactccatctcagcttttcttctcacttcatgtgaaaactactccagtggctgactgaattgctgacccttcaagctctgtccttatccattacctcaaagcagtcattccttagtaaagtttccaacaaatagaaattaatgacactttggtagcactaatatggagattatcctttcattgagccttttatcctctgttctcctttgaagaacccctcactgtcaccttcccgagaataccctaagaccaataaatacttcagtatttc

SEQ ID NO: 33 - DNA, 612 bp, Homo sapiens
tttcaagttttatgatttatttaacttgtggaacaaaaataaaccagattaaccacaaccatgccttactttatcaaatgtataagaagtaaatatgaatcttatatgacaaaatgtttcattcattataacaaatttccaataatcctgtcaattatatttctaaattttcccccaaattctaagcagagtatgtaaattggaagttaacttatgcacgcttaactatcttaacaagctttgagtgcaagagattgaagagttcaaatctgaccaagatgttgatgttggataagagaattctctgctccccacctctaagttgccagccctcctagagctacctgtggagcaacctgctcagatacatcaaacatggagacagcactcaaagtagaattataaagaagatcatgtccatgttaacattattataaccccacattttgtgcctaaagtgtaagtggtatagcctatccatattaaaaaggccagccagcggaatttgggattcatcgcatccacatgcgggagctttcagactcccagagggggcgctacaaggctcggtcccacctacaaatctggcgtggacaagtcccgggttaacgg

SEQ ID NO: 34 - DNA, 925 bp, Homo sapiens
ggcacgagccgagatgtctcgctccgtggccttagctgtgctcgcgctactctctctttctggcctggaggctatccagcgtactccaaagattcaggtttactcacgtcatccagcagagaatggaaagtcaaatttcctgaattgctatgtgtctgggtttcatccatccgacattgaagttgacttactgaagaatggagagagaattgaaaaagtggagcattcagacttgtctttcagcaaggactggtctttctatctcttgtactacactgaattcacccccactgaaaaagatgagtatgcctgccgtgtgaaccatgtgactttgtcacagcccaagatagttaagtgggatcgagacatgtaagcagcatcatggaggtttgaagatgccgcatttggattggatgaattccaaattctgcttgcttgctttttaatattgatatgcttatacacttacactttatgcacaaaatgtagggttataataatgttaacatggacatgatcttctttataattctactttgagtgctgtctccatgtttgatgtatctgagcaggttgctccacaggtagctctaggagggctggcaacttagaggtggggagcagagaattctcttatccaacatcaacatcttggtcagatttgaactcttcaatctcttgcactcaaagcttgttaagatagttaagcgtgcataagttaacttccaatttacatactctgcttagaatttgggggaaaatttagaaatataattgacaggattattggaaatttgttataatgaatgaaacattttgtcatataagattcatatttacttcttatacatttgataaagtaaggcatggttgtggttaatctggtttatttttgttccacaagttaaataaatcataaaacttg

SEQ ID NO: 35 - DNA, 257 bp, Homo sapiens
  misc_feature (1)..(257): n = a, c, g, or t
tagcttcttaaggtccttttcttctgtcttagcttttccaaaattagttccttgactcatctctgctgatgaaacagggaaatcagaggtatccacatcatcgtaaacttcatctcccatgtccaattgtttaggaggagcagggaaaccatcatcagcatcttcctcttcaatcccatcataaatgtcatcatctggtggtggagggaatatccctccacttccactctgactgtggctgctaanatcatcctgcg

SEQ ID NO: 36 - DNA, 2400 bp, Homo sapiens
taggatggaaaggcagatgtaaagtccctcatggcgaaatataacacggggggcaacccgacagaggatgtctcagtcaatagccgacccttcagagtcacagggccaaactcatcttcaggaatacaagcaagaaagaacttattcaacaaccaaggaaatgccagccctcctgcaggacccagcaatgtacctaagtttgggtccccaaagccacctgtggcagtcaaaccttcttctgaggaaaagcctgacaaggaacccaagcccccgtttctaaagcccactggagcaggccaaagattcggaacaccagccagcttgaccaccagagaccccgaggcgaaagtgggatttctgaaacctgtaggccccaagcccatcaacttgcccaaagaagattccaaacctacatttccctggcctcctggaaacaagccatctcttcacagtgtaaaccaagaccatgacttaaagccactaggcccgaaatctgggcctactcctccaacctcagaaaatgaacagaagcaagcgtttcccaaattgactggggttaaagggaaatttatgtcagcatcacaagatcttgaacccaagcccctcttccccaaacccgcctttggccagaagccgcccctaagtaccgagaactcccatgaagacgaaagccccatgaagaatgtgtcttcatcaaaagggtccccagctcccctgggagtcaggtccaaaagcggccctttaaaaccagcaagggaagactcagaaaataaagaccatgcaggggagatttcaagtttgccctttcctggagtggttttgaaacctgctgcgagcaggggaggcctaggtctctccaaaaatggtgaagaaaaaaaggaagataggaagatagatgctgctaagaacaccttccagagcaaaataaatcaggaagagttggcctcagggactcctcctgccaggttccctaaggccccttctaagctgacagtgggggggccatggggccaaagtcaggaaaaggaaaagggagacaagaattcagccaccccgaaacagaagccattgcctcccttgtttaccttgggtccacctccaccaaaacccaacagaccaccaaatgttgacctgacgaaattccacaaaacctcttctggaaacagtactagcaaaggccagacgtcttactcaacaacttccctgccaccacctccaccatcccatccggccagccaaccaccattgccagcatctcacccatcacaaccaccagtcccaagcctacctcccagaaacattaaacctccgtttgacctaaaaagccctgtcaatgaagacaatcaagatggtgtcacgcactctgatggtgctggaaatctagatgaggaacaagacagtgaaggagaaacatatgaagacatagaagcatccaaagaaagagagaagaaaagggaaaaggaagaaaagaagaggttagagctggagaaaaaggaacagaaagagaaagaaaagaaagaacaagaaataaagaagaaatttaaactaacaggccctattcaagtcatccatcttgcaaaagcttgttgtgatgtcaaaggaggaaagaatgaactgagcttcaagcaaggagagcaaattgaaatcatccgcatcacagacaacccagaaggaaaatggttgggcagaacagcaaggggttcatatggctatattaaaacaactgctgtagagattgactatgattctttgaaactgaaaaaagactctcttggtgccccttcaagacctattgaagatgaccaagaagtatatgatgatgttgcagagcaggatgatattagcagccacagtcagagtggaagtggagggatattccctccaccaccagatgatgacatttatgatgggattgaagaggaagatgctgatgatggtttccctgctcctcctaaacaattggacatgggagatgaagtttacgatgatgtggatacctctgatttccctgtttcatcagcagagatgagtcaaggaactaattttggaaaagctaagacagaagaaaaggaccttaagaagctaaaaaagcaggaaaaagaagaaaaagacttcaggaaaaaatttaaatatgatggtgaaattagagtcctatattcaactaaagttacaacttccataacttctaaaaagtggggaaccagagatctacaggtaaaacctggtgaatctctagaagttatacaaaccacagatgacacaaaagttctctgcagaaatgaagaagggaaatatggttatgtccttcggagttacctagcggacaatgatggagagatctatgatgatattgctgatggctgcatctatgacaatgactagcactcaactttggtcatt

SEQ ID NO: 37 - DNA, 270 bp, Homo sapiens
  misc_feature (1)..(270): n is a, c, g, or t
catcaaaatagtttattgtgttcagaaaaatcacagtttgaggaaaccttaaacaataagtacacaagagatgccccatggnattnccatagcagagacagactcctgtatgttttattccagaggcattgcatggngataataaaatgataggaantagaggaaantagatacaggaaaaggcantagacagggangccagctagatgttagagtacggagcattcgaggaggcntaaccacanttgggggtggctttagggctgggaa

SEQ ID NO: 38 - DNA, 1237 bp, Homo sapiens
attcttgtctgttctgcctcactcccgagctctactgactcccaaaagagcgcccaagaagaaaatggccataagtggagtccctgtgctaggatttttcatcatagctgtgctgatgagcgctcaggaatcatgggctatcaaagaagaacatgtgatcatccaggccgagttctatctgaatcctgaccaatcaggcgagtttatgtttgactttgatggtgatgagattttccatgtggatatggcaaagaaggagacggtctggcggcttgaagaatttggacgatttgccagctttgaggctcaaggtgcattggccaacatagctgtggacaaagccaacctggaaatcatgacaaagcgctccaactatactccgatcaccaatgtacctccagaggtaactgtgctcacgaacagccctgtggaactgagagagcccaacgtcctcatctgtttcatcgacaagttcaccccaccagtggtcaatgtcacgtggcttcgaaatggaaaacctgtcaccacaggagtgtcagagacagtcttcctgcccagggaagaccaccttttccgcaagttccactatctccccttcctgccctcaactgaggacgtttacgactgcagggtggagcactggggcttggatgagcctcttctcaagcactgggagtttgatgctccaagccctctcccagagactacagagaacgtggtgtgtgccctgggcctgactgtgggtctggtgggcatcattattgggaccatcttcatcatcaagggagtgcgcaaaagcaatgcagcagaacgcagggggcctctgtaaggcacatggaggtgatgatgtttcttagagagaagatcactgaagaaacttctgctttaatgactttacaaagctggcaatattacaatccttgacctcagtgaaagcagtcatcttcagcgttttccagccctatagccaccccaagtgtggttatgcctcctcgattgctccgtactctaacatctagctggctttccctgtctattgccttttcctgtatctattttcctctatttcctatcattttattatcaccatgcaatgcctctggaataaaacatacaggagtctgtctctgctatggaatgccccatggggctctcttgtgtacttattgtttaaggtttcctcaaactgtgatttttctgaacacaataaactattttgatgatcttgggtggaaaa

SEQ ID NO: 39 - DNA, 456 bp, Homo sapiens
  misc_feature (1)..(456): n = a, c, g, or t
tcctcagtagtttgaacacttgctggctattttttctgtccaagttctcagtaacttcggcctgtgtagtcagtggttctacacagccgacactacttcttacataacacttggtctctctggcttctggaaagggcgaggggttaccttccggagtccagtgctctttcggcacttctgcaaccaggcagtggtaaaaggggtgctctccaaaaccatcttctagcagatctttttcagttaagattacttgttcttccatgtattcatatttaagccagctccttgatcagccgcagtatgtcactgcagtcggcggcagtggctgggcggatcaccgaatttagccattttcggtctttttgctttttcttccctttgcgggaccagggccccctggtacttgaacagtaggaggaaggtgggttccncaatcggtctcccggggangcggtn

SEQ ID NO: 40 - DNA, 1060 bp, Homo sapiens
cgcgggccgactggtgtttatccgtcactcgccgaggttccttgggtcatggtgccagcctgactgagaagaggacgctcccgggagacgaatgaggaaccacctcctcctactgttcaagtacaggggcctggtccgcaaagggaagaaaagcaaaagacgaaaatggctaaattcgtgatccgcccagccactgccgccgactgcagtgacatactgcggctgatcaaggagctggctaaatatgaatacatggaagaacaagtaatcttaactgaaaaagatctgctagaagatggttttggagagcaccccttttaccactgcctggttgcagaagtgccgaaagagcactggactccggaaggacacagcattgttggttttgccatgtactattttacctatgacccgtggattggcaagttattgtatcttgaggacttcttcgtgatgagtgattatagaggctttggcataggatcagaaattctgaagaatctaagccaggttgcaatgaggtgtcgctgcagcagcatgcacttcttggtagcagaatggaatgaaccatccatcaacttctataaaagaagaggtgcttctgatctgtccagtgaagagggttggagactgttcaagatcgacaaggagtacttgctaaaaatggcaacagaggagtgaggagtgctgctgtagatgacaacctccattctattttagaataaattcccaacttctcttgctttctatgctgtttgtagtgaaataatagaatgagcacccattccaaagctttattaccagtggcgttgttgcatgtttgaaatgaggtctgtttaaagtggcaatctcagatgcagtttggagagtcagatctttctccttgaatatctttcgataaacaacaaggtggtgtgatcttaatatatttgaaaaaaacttcattctcgtgagtcatttaaatgtgtacaatgtacacactggtacttagagtttctgtttgattcttttttaataaactactctttgatttaaaaaaaaaaaaaaaaaaaaaaaaaaa

SEQ ID NO: 41 - DNA, 522 bp, Homo sapiens
  misc_feature (1)..(522): n is a, c, g, or t
cggccgcgtnttgaaggatgacctctaggaagaaagtgttgctgaaggttatcatcctgggagattctggagtcgggaagacatcactcatgaaccagtatgtgaataagaaattcagcaatcagtacaaagccacaataggagctgactttctgaccaaggaggtgatggtggatgacaggctagtcacaatgcagatatgggacacagcaggacaggaacggttccagtctctcggtgtggccttctacagaggtgcagactgctgcgttctggtatttgatgtgactgcccccaacacattcaaaaccctagatagctggagagatgagtttctcatccaggccagtccccgagatcctgaaaacttcccatttgttgtgttgggaaacaagattgacctcgaaaacagacaagtggccacaaagcgggcacaggctggtgctacagcaaaaacaacattccctactttgagaccagtgccaaggaggccatcaacgtggagcaggcgttccagacgat

SEQ ID NO: 42 - DNA, 1498 bp, Homo sapiens
ggcacgaggataaagcctgaggcggcggcagcggcggagttggcggcttggagagctcgggagagttccctggaaccagaacttggaccttctcgcttctgtcctccgtttagtctcctcctcggcgggagccctcgcgacgcgcccggcccggagcccccagcgcagcggccgcgtttgaaggatgacctctaggaagaaagtgttgctgaaggttatcatcctgggagattctggagtcgggaagacatcactcatgaaccagtatgtgaataagaaattcagcaatcagtacaaagccacaataggagctgactttctgaccaaggaggtgatggtggatgacaggctagtcacaatgcagatatgggacacagcaggacaggaacggttccagtctctcggtgtggccttctacagaggtgcagactgctgcgttctggtatttgatgtgactgcccccaacacattcaaaaccctagatagctggagagatgagtttctcatccaggccagtccccgagatcctgaaaacttcccatttgttgtgttgggaaacaagattgacctcgaaaacagacaagtggccacaaagcgggcacaggcctggtgctacagcaaaaacaacattccctactttgagaccagtgccaaggaggccatcaacgtggagcaggcgttccagacgattgcacggaatgcacttaagcaggaaacggaggtggagctgtacaacgaatttcctgaacctatcaaactggacaagaatgaccgggccaaggcctcggcagaaagctgcagttgctgagggggcagtgagagttgagcacagagtccttcacaaaccaagaacacacgtaggccttcaacacaattcccctctcctcttccaaacaaaacatacattgatctctcacatccagctgccaaaagaaaaccccatcaaacacagttacaccccacatatctcgcacacacacacacacacgcacacacacacacacagatctgacgtaatcaaactccagcccttgcccgtgatggctccttggggtctgcctgcccacccacatgagcccgcgagtatggcagcaggacaagccagcggtggaagtcattctgatatggagttggcattggaagcttattctttttgttcactggagagagagagaactgtttacagttaatctgtgtctaattatctgattttttttattggtcttgtggtctttttaccccccctttcccctccctccttgaaggctaccccttgggaaggctggtgccccatgccccattacaggctcacacccagtctgatcaggctgagttttgtatgtatctatctgttaatgcttgttacttttaactaatcagatctttttacagtatccatttattatgtaatgcttcttagaaaagaatcttatagtacatgttaatatatgcaaccaattaaaatgtataaattagtgtaaaaaaaaaaaaaaaaaaa

SEQ ID NO: 43 - DNA, 413 bp, Homo sapiens
tttaacgtttgcaaaggtttatttactgtcagtgactgctacaggggaatcggatgaggggagtacatgctgaccaggaaacagagtgagggaagcctgaccaggacgcatggcaatgggaaaagcagatgggagatgcttatactggtacttggtgtgtgtgtgtgtgtgtgtgtgtgtgtgtgtggtgtggtttgtgtgtgtgtgtaaatgcagaggaaaaaatctgaaattaaacactcagaactgccctcagtagtcacatctagggagagaggagggtaatgctgttccatgcagaggtaactgacaatacttgttttctaaggtaggtgcatggatacacaaaccaaaatatgcattaagtatgtcttgctcatcagtgaaaatgttaatatctaacagaatgacac

SEQ ID NO: 44 - DNA, 1290 bp, Homo sapiens
ctctctctttcgattcttccatactcaagagtacgcacggtctgattttctctttggattcttccaaaatcagagtcagactactccctgtgccatgaacggagatgacacctttgcaaggagacccacggttggtgctcaaataccagagaagatacaaaaggccttcgatgatattgccaaatacttctctaaggaagagtgggaaaagatgaaagtctcggagaaaatcgtctatgtgtatatgaagagaaagtatgaggccatgactaaactaggtttcaaggccatcctcccatctttcatgcgtaataaacgggtcacagacttccaggggaatgattttgataatgaccctaaccgtgggaatcaggttcaacgtcctcagatgactttcggcaggctccagggaatcttcccgaagatcatgcccaagaagccagcagaggaaggaaatgtttcgaaggaagtgccagaagcatctggcccacaaaacgatgggaaacagctgtgccccccgggaaaaccaactacctctgagaagattaacatgatatctggacccaaaaggggggaacatgcctggacccacagactgcgtgagagaaagcagctggtgatttatgaagagatcagcgatcctgaggaagatgatgagtaactccccttggggatatgacacatgcccatgatgagaagcagaacgtggtgacctttcacgaacatgggcatggctgtggacccctcgtcatcaggtgcatagcaagtgaaagcaagtgttcacaacagtgaaaagttgagcgtcatttttcttagtgtgccaagagtacgatattagcgtttccattgtattttcttgaagtgtgtcattctgttagatattaacattttcactgatgagcaagacatacttaatgcatattttggtttgtgtatccatgcacctaccttagaaaacaagtattgtcagttacctctgcatggaacagcattaccctcctctctccctagatgtgactactgagggcagttctgagtgtttaatttcagattttttcctctgcatttacacacacacacaaaccacaccacacacacacacacacacacacagacacacaccaagtaccagtataagcatctcccatctgcttttcccattgccatgcgtcctggtcaggcttccctcactctgtttcctggtcagcatgtactcccctcatccgattcccctgtagcagtcactgacagtaaataaacctttgcaaacgttaaaaaaaaaaaaaaaaa

SEQ ID NO: 45 - DNA, 467 bp, Homo sapiens
acttttagaaactgatgtttattttccatcaaccatttttccatgctgcttaagagcctatgcaagaacagcttaagaccagtcagtggttgctcctacccattcagtggcctgagcagtgggagctgcagaccagtcttccgtggcaggctgagcgctccagtcttcagtagggaattgctgaataggcacagagggcacctgtacaccttcagaccagtctgcaacctcaggctgagtagcagtgaactcaggagcgggacgagtccattcaccctgaaattcctccttggtcactgccttctcagcagcagcctgctcttctttttcaatctcttcaggatctctgtagaagtacagatcaggcatgacctcccatgggtgttcacgggaaatggtgccacgctgcgcagaacttcccgagccagcatccaccacatcaaacccactgagtgagatcccttgtt

SEQ ID NO: 46 - DNA, 1039 bp, Homo sapiens
tgcctgtcttttccgtgctacctgcagaggggtccatacggcgttgttctggattcccgtcgtaacttaaagggaaactttcacaatgtccggagcccttgatgtcctgcaaatgaaggaggaggatgtccttaagttccttgcagcaggaacccacttaggtggcaccaatcttgacttccagatggaacagtacatctataaaaggaaaagtgatggcatctatatcataaatctcaagaggacctgggagaagcttctgctggcagctcgtgcaattgttgccattgaaaaccctgctgatgtcagtgttatatcctccaggaatactggccagagggctgtgctgaagtttgctgctgccactggagccactccaattgctggccgcttcactcctggaaccttcactaaccagatccaggcagccttccgggagccacggcttcttgtggttactgaccccagggctgaccaccagcctctcacggaggcatcttatgttaacctacctaccattgcgctgtgtaacacagattctcctctgcgctatgtggacattgccatcccatgcaacaacaagggagctcactcagtgggtttaatgtggtggatgctggctcgggaagttctgcgcatgcgtggcaccatttcccgtgaacacccatgggaggtcatgcctgatctgtacttctacagagatcctgaagagattgaaaaagaagagcaggctgctgctgagaaggcagtgaccaaggaggaatttcagggtgaatggactgctcccgctcctgagttcactgctactcagcctgaggttgcagactggtctgaaggtgtacaggtgccctctgtgcctattcagcaattccctactgaagactggagcgctcagcctgccacggaagactggtctgcagctcccactgctcaggccactgaatgggtaggagcaaccactgactggtcttaagctgttcttgcataggctcttaagcagcatggaaaaatggttgatggaaaataaacatcagtttct

SEQ ID NO: 47 - DNA, 422 bp, Homo sapiens
ttgcagtttgaatcctggtttatttaagtagcattcaggagttatactgtaaataaacatcattatggctaatgactaattaaaatcaccattcattacaaataccaaccaggttagataatactattaactatcttttccccttcggtctggggttcatattcttccttttctggaagtcattttcatggataaaatatttagcattttttgttacctcttccttcaccaactgtactcaacacacttctgtaaacagagtgagaacctgggttcatggacaagctcttttccacttatcttcgggttaaaccaaaactttttcagcaactttgcccctgtccaagttttgcagaacaccagctgcctcttcctgttctaggcattctagggctagatacctgtgaacggacttcaagcat

SEQ ID NO: 48 - DNA, 4654 bp, Homo sapiens
agtaattgctgtaccaaggtcagaatcgccacctgaggcctgaatatcagcgtaagatagtgtccaaagcagtcttaagaagaggtcccattaccccactctttccgccctaatggaggtctccagtttagcctcacagcatggaaccacaggttactctaaatgtgacttttaaaaatgaaattcaaagctttctggtttctgatccagaaaatacaacttgggctgatatcgaagctatggtaaaagtttcatttgatctgaatactattcaaataaaatacctggatgaggaaaatgaagaggtatccatcaacagtcaaggagaatatgaagaagcgcttaagatggcagttaaacagggaaaccaactgcagatgcaagtccacgaagggcaccatgtcgttgatgaagccccacccccagttgtaggagcaaaacgactagctgccagggcagggaagaagccacttgcacattactcttcactggtgagagtcttgggatcagacatgaagaccccagaggatcctgcagtgcagtcgtttccacttgttccatgtgacacagaccagcctcaagacaagcccccagactggttcacaagctacctggagacgttcagagaacaagtggttaacgaaacggttgagaagcttgaacagaaattacatgaaaagcttgtcctccagaacccatccttgggttcttgtccctcagaagtctcaatgcctacttcagaagaaacattgtttttgccagaaaaccagttcagctggcatattgcttgcaacaactgccaaagaaggattgttggtgtccgctaccagtgtagcctatgcccatcctacaatatctgtgaagattgtgaagcagggccatatggccatgacactaaccacgtcctgctgaagttgcggagacctgttgtgggctcctctgaaccgttctgtcactcaaagtactctactcctcgtcttcctgctgctctggaacaagtcaggctccagaaacaggttgataagaactttcttaaagcagaaaagcaaaggttgcgagctgagaagaaacaacgtaaagcagaggtcaaggaacttaaaaagcagcttaaactccataggaaaattcacctgtggaattcaatccatggactccagagccccaagtctcctttaggccgacctgagagcttgctccagtctaataccctgatgctccctttgcagccctgtacctccgttatgccaatgctcagtgcagcatttgtggatgagaatttgcctgatgggactcaccttcagccaggaaccaagtttatcaaacactggaggatgaaaaatacaggaaatgtaaagtggagtgcagacacaaagctcaagttcatgtggggaaacctgactttggcttccacagaaaagaaggatgttttggttccctgcctcaaggccggccatgtgggagttgtatctgtggagttcattgccccagccttggagggaacgtatacttcccattggcgtctttctcacaaaggccagcaatttgggcctcgggtctggtgcagtatcatagtagatcctttcccctccgaagagagccctgataacattgaaaagggcatgatcagctcaagcaaaactgatgatctcacctgccagcaagaggaaacttttcttctggctaaagaagaaagacagcttggtgaagtgactgagcagacagaagggacagcagcctgcatcccacagaaggcaaaaaatgttgccagtgagagggagctctacatcccatctgtggatcttctgactgcccaggacctgctgtcctttgagctgttggatataaacattgttcaagagttggagagagtgccccacaacacccctgtggatgtgactccctgcatgtctcctctgccacatgacagtcctttaatagagaagccaggcttggggcagatagaggaagagaatgaaggggcaggatttaaagcacttcctgattctatggtgtcagtaaagaggaaggctgagaacattgcttctgtggaggaagcagaagaagacctgagtgggacccagtttgtgtgtgagacagtaatccgatcccttaccttggatgctgccccagaccacaaccctccttgcagacagaagtccttgcagatgacatttgccttgcctgaaggaccacttggaaatgagaaggaggagattatccatatcgctgaggaagaagctgtcatggaggaggaggaggatgaggaggatgaggaggaggaggatgagctcaaagatgaagttcaaagtcagtcctctgcttcctcagaggattacatcatcatcctgcctgagtgctttgataccagccgccccctgggggattctatgtacagctctgcgctctcacagccaggcctggagcgaggtgctgaaggcaagcctggggttgaggctgggcaggaaccagctgaggctggggaaagactccctggaggggagaaccagccacaggagcacagcataagtgacatcctcacgacctcacagactctggaaacagtgcccctaatcccagaggtagtggagcttccaccgtcactgcccaggagctctccttgtgtacatcatcatggttccccaggagtggatttaccagttaccataccagaagtttcttcagtccctgatcagatcagaggagagcccagaggctcatcaggacttgtaaacagcagacagaagagctatgaccactcaaggcaccatcatgggagcagcattgctggaggactggtgaagggggctttgtctgttgctgcctctgcatacaaggccctgtttgctgggccaccagtcactgcacagccaataatttctgaagatcagacagcagccctgatggcccatctctttgaaatgggattctgtgacaggcagctgaacctacggctgctgaagaaacacaattacaatatcctgcaggttgtgacagaacttcttcagttaaacaacaacgactggtacagccaacgctattgaggagtgaccttgtattaaataactgcctgctgctcagagatgatctttattctgtcattggggtatgggatagaagcccttgcttatttttaatctgatgaatctgtatagagcccatcgttgagttaccaagacaatacctgctacagtattttggggagcaaactaaagaccagaacttaaattttcactttagacattggatgaatagtatgaagacagtttttcagttgatttggataaaactattttagtgcattgacaagtgtaacttcaacttcatatagaaccatttttctttctgcttttattgaaactgagtatttttctttggctaatgtggattttttatggggatatctgttaattttcaggttttgaaagacattaacctcggaagttgtttttaagaattattctcataattcttattctcataatttctgtaatccacctcaagcttcatagttatttggcattgaaataacacccagagcatgatagaaatgttgttactcttcctctctcaaggagaaagtaattttcctgcaatacttaataattggcaccgttgctttctaaagactccatggtgcattcaagagtatccaacttcaagggaatctccgcatttcaatgaaaggaggaagagtgtgctgataaacctaccagcacctattgagcaatgtctattatagtaattttgcatacatttttatttaagggaaaaaatataggtattgtgaaatattttgctaatcttatagaaaaggaaaaaatcccgttatttaaagggaaaagtaaatttaacagttgccttttttcttaatgtcagggcagatcttattttacagtacagtgggggaaatagaaaacatgtgaaaggcaaaaggcaggctcctaaattaatgtcagtgaagttcagggtgggcaaatgagtgtgtgtgaggtataggaaatgctgatgacttctttaatgcttgaagtccgttcacaggtatctagccctagaatgcctagaacaggaagaggcagctggtgttctgcaaaacttggacaggggcaaagttgctgaaaaagttttggtttaacccgaagataagtggaaaagagcttgtccatgaacccaggttctcactctgtttacagaagtgtgttgagtacagttggtgaaggaagaggtaacaaaaaatgctaaatattttatccatgaaaatgacttccagaaaaggaagaatatgaaccccagaccgaaggggaaaagatagttaatagtattatctaacctggttggtatttgtaatgaatggtgattttaattagtcattagccataatgatgtttatttacagtataactcctgaatgctacttaaataaaccaggattcaaactgcaagccagccaggccgttcattatttaaaacgttttaatcggggcttccgggtagaaggtggagcggcagggtgtaattgggttgatgggtgggacctgtcttgaccatcggagttttataatcgagggccaggagggcccgggttgctctcctggttatgtatgtacttgtacataaccaccttaagaatggtgaaataaatgttcttggaaattcc

SEQ ID NO: 49 - DNA, 394 bp, Homo sapiens
  misc_feature (1)..(394): n is a, c, g, or t
ggtgggatgnaataanaagaagaggccaaaacttcaaatccaacttcttcactaacttctctttctgtggcaccaacattagcccaaatataactctgggacccacctatttaaccactgtcaattcttcagactctgacaatgggaccacaagaacagcaagcaccaattctataggcattacaatttcaccaaatggaacgtgggcttctagataaccagttcacggatgccagaacagaaccctgggagggganttccagcaccgcaggcaaccactccagaaactttccctncttcaggtaattctgactcganggacagnngnggtgagctgctcacctttatatctgttgttccttttacacagtgtacagtntttcatttnttttcc

SEQ ID NO: 50 - DNA, 3643 bp, Homo sapiens
ggagtatgagtcacgctcaggggagcaggtaccccttctcctaaagatgaagaggagcaaactggcactaagcaaggccatcgagagcggggacactgacctggtgttcacggtgttgctgcacctgaagaacgagctgaaccgaggagattttttcatgacccttcggaatcagcccatggccctcagtttgtaccgacagttctgtaagcatcaggagctagagacgctgaaggacctttacaatcaggatgacaatcaccaggaattgggcagcttccacatccgagccagctatgctgcagaagagcgtattgaggggcgagtagcagctctgcagacagccgccgatgccttctacaaggccaagaatgagtttgcagccaaggctacagaggatcaaatgcggctcctacggctgcagcggcgcctagaagacgagctggggggccagttcctagacctgtctctacatgacacagttaccaccctcattcttggcggtcacaacaagcgtgcagagcagctggcacgtgacttccgcatccctgacaagaggtgacacaactaaaaaaaaacaaaggtatttatggaattccactgagtggtaatggatgatgcagttcaaataactaaggacacatgttcaaagagcataattaactttttaaaagaagctaataagcatggattcctggttcattcttgttctgctcggcagtggtctgatatgtgtcagtgccaacaatgctaccacagttgcaccttctgtaggaattacaagattaattaactcatcaacggcagaaccagttaaagaagaggccaaaacttcaaatccaacttcttcactaacttctctttctgtggcaccaacattcagcccaaatataactctgggacccacctatttaaccactgtcaattcttcagactctgacaatgggaccacaagaacagcaagcaccaattctataggcattacaatttcaccaaatggaacgtggcttccagataaccagttcacggatgccagaacagaaccctgggaggggaattccagcaccgcagcaaccactccagaaactttccctccttcaggtaattctgactcgaaggacagaagagatgagacaccaattattgcggtgatggtggccctgtcctctctgctagtgatcgtgtttattatcatagttttgtacatgttaaggtttaagaaatacaagcaagctgggagccattccaattctttccgcttatccaacggccgcactgaggatgtggagccccagagtgtgccacttctggccagatccccaagcaccaacaggaaatacccacccctgcccgtggacaagctggaagaggaaattaaccggagaatggcagacgacaataagctcttcagggaggaattcaacgctctccctgcatgtcctatccaggccacctgtgaggctgcttccaaggaggaaaacaaggaaaaaaatcgatatgtaaacatcttgccttatgaccactctagagtccacctgacaccggttgaaggggttccagattctgattacatcaatgcttcattcatcaacggttaccaagaaaagaacaaattcattgctgcacaaggaccaaaagaagaaacggtgaatgatttctggcggatgatctgggaacaaaacacagccaccatcgtcatggttaccaacctgaaggagagaaaggagtgcaagtgcgcccagtactggccagaccaaggctgctggacctatgggaatattcgggtgtctgtagaggatgtgactgtcctggtggactacacagtacggaagttctgcatccagcaggtgggcgacatgaccaacagaaagccacagcgcctcatcactcagttccactttaccagctggccagactttggggtgccttttaccccgatcggcatgctcaagttcctcaagaaggtgaaggcctgtaaccctcagtatgcaggggccatcgtggtccactgcagtgcaggtgtagggcgtacaggtacctttgtcgtcattgatgccatgctggacatgatgcatacagaacggaaggtggacgtgtatggctttgtgagccggatccgggcacagcgctgccagatggtgcaaaccgatatgcagtatgtcttcatataccaagcccttctggagcattatctctatggagatacagaactggaagtgacctctctagaaacccacctgcagaaaatttacaacaaaatcccagggaccagcaacaatggattagaggaggagtttaagaagttaacatcaatcaaaatccagaatgacaagatgcggactggaaaccttccagccaacatgaagaagaaccgtgttttacagatcattccatatgaattcaacagagtgatcattccagttaagcggggcgaagagaatacagactatgtgaacgcatcctttattgatggctaccggcagaaggactcctatatcgccagccagggccctcttctccacacaattgaggacttctggcgaatgatctgggagtggaaatcctgctctatcgtgatgctaacagaactggaggagagaggccaggagaagtgtgcccagtactggccatctgatggactggtgtcctatggagatattacagtggaactgaagaaggaggaggaatgtgagagctacaccgtccgagacctcctggtcaccaacaccagggagaataagagccggcagatccggcagttccacttccatggctggcctgaagtgggcatccccagtgacggaaagggcatgatcagcatcatcgccgccgtgcagaagcagcagcagcagtcagggaaccaccccatcaccgtgcactgcagcgccggggcaggaaggacggggaccttctgtgccctgagcaccgtcctggagcgtgtgaaagcagaggggattttggatgtcttccagactgtcaagagcctgcggctacagaggccacacatggtccagacactggaacagtatgagttctgctacaaggtggtgcaggagtatattgatgcattctcagattatgccaacttcaagtaagcggcaacaagggtccgtggaccaggaggattgcctttaatattttgtaatattctgttttgttaatataccccaaattgtgtatatatcttataactgttttagaaattggtacataggcttctattacctattaggtggaaattttatatgtaaatgtgttagcactgatagtcctttttccaatgttttattggggaattaaatagtgtgatgtttggattgatatcgtgaaatcctcagccgagaaattgggctggattgtgctttggttaatacatctttccctaaagaagataaacacaaaatccattccaggtagctcggcaccaactaagaaaaaaagcacaaagttctcagagctctcgaggaaagtggttgtccccgtaccaccatgcactgtaaatatccctcccctctctccctggtcccctcccccatccccaccactgatatcatggggagtaataggaccagagcggtatctctggcaccacactagggactatcaggtaataaaagctttgactccctgaaaaaaaaaaaaa

SEQ ID NO: 51 - DNA, 502 bp, Homo sapiens
  misc_feature (1)..(502): n = a, c, g, or t
tttttttttaatggtcttttttttttttttaatgtttcttcctctgtttggctttgcatttgtgcgatttggaaaaaccacttggaagaagggactttcctgcaaaaccttaaagactggttaaattacagggcctaggaagtcagtggagccccttgactgacaaagcttagaaaggaactgaaattgcttctttgaatatggattttagggcggggcgtgggtgggctcacgccttattaatcccaggcacgttggggagggccaacgcggggtgggatcacctgaggttcagggagtttcggaggataacgacagagggccccgggcacttcacagttcaccttcattcagtttccttcacttatccctctggcaggggaacaggacantcttactgttccttgtgttgcagttacttcctntttatctttnccacagttttgantcctcccntttcccntccacaggggttntttttcccgggggtaccatnggtggg

SEQ ID NO: 52 - DNA, 1809 bp, Homo sapiens
aatccagaggcaggggagggacggtgcaggcgcagagtattgggtttggctggcctcgatttaaagagacagaagctgtcggggtcctggaagacggtccccaataccctccccccaagtccttgggaccacttgggtccccagagctggggagatggtttgtggcggctttgcctgctccaagaatgcgctttgcgctctcaacgtggtctacatgctggtgagcttgttgctcattggagtggctgcttggggcaagggcctgggtctggtgtccagcatccacatcatcggcggagtcattgctgtgggagtcttccttctccttattgcagtggctggactggtgggtgctgtcaaccaccaccaagtcctgctgttcttttacatgatcatccttggtttggtcttcatcttccaatttgtaatctcttgctcatgtctggctattaaccgaagcaaacagacagatgtcatcaatgcttcttggtgggtcatgagcaacaagactcgggatgaactggaaagaagttttgattgttgtggcttattcaacctcacaaccctgtatcaacaagattatgatttctgcactgcaatctgcaagagccagagccccacatgccagatgtgtggagaaaagtttcttaagcattcagacgaagccctgaaaatcctagggggtgttggactcttctttagctttacagagatccttggtgtttggctagcaatgagatttcggaatcagaaggatcctagagccaaccccagtgcctttctatgagactttggatccttctgacttttcttctgctctctctaagctttctcttcctcccttagggaatatctagggtctgtaaccgttttggtttgagaaaaaggaaaggccccttgtcacatcctctaaaattgatggaatagcaagactttatgccttgacatattttagtgggagccagactataaggaataaaaggaaaaactttcttcctctctctccaagaggatatgggaagcttctgtgagtgcataggatgggggctggagtcattcttagctgtttcccttcctctgtccatatactggatcacctcaacataccctggtgtggctctaagggtaaatcagggatagggccaaggagaaaacaaccaagaactctttcctgtaataagcaggatccagtttgagaaagtttagcgaatataaaagtaaaagccatttaaaaatctatattctttttttttttgacacagagtcttgctctgttgcccaggctggagtgcaatggcatgatctcggctcaccgcaacctctgcctcccgggttcaagcgatcctcctgcctcagtctcccaagtagctgggattacaggtgtgcaccaccacgcctgactaattttgtatttttagtagagacagggtttcaccatgttagtcaggctggtctcgaactcctgacctcaggtgatccacccgcgttggcctcccaacgtgctgggattataggcgtgagccaccacgccccgcctaaaatccatattcaaagaagcaatttcagttcctttctaagctttgtcagtcaaggggctccactgacttcctaggccctgtaatttaaccagtctttaaggttttgcaggaaagtcccttcttccaagtggtttttccaaatcgcacaatggcaaagccaaacagaggaagaaacattaaaaaaaaaaaaaagaccattaaaaaaaaaaaaaaaaaaaaaaaaaaaaaaaaaaaaaaaaaaaaaaaaaaaaaaaaaaaaaaaaaaaaaaaa

SEQ ID NO: 53 - DNA, 602 bp, Homo sapiens
  misc_feature (1)..(602): n is a, c, g, or t
ttttttttctcttttcattttttattaaggcagtaacattctttcttcctggnggcggaggggggaaatcatctgaggctacagtaagttcaatctgaagtcaaaaccaaccaatttgtattcagaagcagcaaaaatactggttatgggctgggaggagccacatgttggctttctgagtccactgccaaggaacagatccagagcaatgcatttcaattgctttgaaaaatggctaagttccattacataccaccactaaagaagatacctcattgatggagcttttcaccatgatagagggcgtcttgcattggttcacaatggcctgttgctttttagccttcagttggtttagggggaaattgctcaacctatgagacctgctcaaattcatactgttctggttttgctttggaattctttgtccagtaaattagctcttcataacgcagttccaccatttcttatccagcatagattcattctgcccttgacagctggctaggtgtgtgtgtatgtgagtaagggtgcattgantggcacaattgtccccctggagttaggggtggcanaanctttatagtaccaaaaagtaacat

SEQ ID NO: 54 - DNA, 3723 bp, Homo sapiens
tttttctttcctggctgatgatttgtcattctagtcacttcctgccttgtgaccacacacccaggcttgacaaagctgttctgcagatcagaaagaaggggttcctggtcatacaccagtactaccaaggacagcttttttcctgcaagatctgttacctaaagcaataaaaaatggccagaggatcagtgtccgatgaggaaatgatggagctcagagaagcttttgccaaagttgatactgatggcaatggatacatcagcttcaatgagttgaatgacttgttcaaggctgcttgcttgcctttgcctgggtatagagtacgagaaattacagaaaacctgatggctacaggtgatctggaccaagatggaaggatcagctttgatgagtttatcaagattttccatggcctaaaaagcacagatgttgccaagacctttagaaaagcaatcaataagaaggaagggatttgtgcaatcggtggtacttcagagcagtctagcgttggcacccaacactcctattcagaggaagaaaagtatgcctttgtcaactggataaacaaagccctggaaaatgatcctgattgtcggcatgtcatcccaatgaacccaaacacgaatgatctctttaatgctgttggagatggcattgtcctttgtaaaatgatcaacctgtcagtgccagacacaattgatgaaagaacaatcaacaaaaagaagctaacccctttcaccattcaggaaaatctgaacttggctctgaactctgcctcagccatcgggtgccatgtggtcaacataggggctgaggacctgaaggaggggaagccttatctggtcctgggacttctgtggcaagtcatcaagattgggttgtttgctgacattgaactcagcagaaatgaagctctgattgctcttttgagagaaggtgagagcctggaggatttgatgaaactctcccctgaagagctcttgctgaggtgggctaattaccacctggaaaatgcaggctgcaacaaaattggcaacttcagtactgacatcaaggactcaaaagcttattaccacctgcttgagcaggtggctccaaaaggagatgaagaaggtgttcctgctgttgttattgacatgtcaggactgcgggagaaggatgacatccagagggcagaatgcatgctgcagcaggcggagaggctgggctgccggcagtttgtcacagccacagatgttgtccgagggaaccccaagttgaacttggcttttattgccaacctctttaacagataccctgccctgcacaaaccagagaaccaggacattgactggggggctcttgaaggtgagacgagagaagagcggacatttaggaactggatgaactccctgggtgttaaccctcgagtcaatcatttgtacagtgacttatcagatgccctggtcatcttccagctctatgaaaagatcaaagttcctgttgactggaacagagtaaacaaaccgccataccccaaactgggaggcaatatgaagaagcttgagaattgtaactacgcggtagaattggggaagaatcaagcgaagttctccctggttggcatcggtggacaagatctcaatgaaggaaaccgcactctcacactggccttgatttggcagctaatgagaaggtatacactgaatatcctcgaagaaattggtggtggccagaaggtcaatgatgacattattgtcaactgggtgaatgaaacattgagggaagcagagaaaagttcatccatctctagtttcaaggacccgaagattagtacaagtctgcctgttctggacctcatcgatgccatccaaccaggttccattaactatgaccttctgaagacagaaaatctgaatgatgatgagaaactcaacaatgcaaaatatgccatctctatggcccgaaaaattggagcaagagtgtatgccctgccagaagacctggttgaagtgaaccccaaaatggtcatgaccgtgtttgcctgcctcatggggaaaggaatgaagagggtgtgaggccaatggggctgggtgggaggcggtgcactcactcctgactgcccggcacagatgctccagggatgattcaagccattccaaagttcaacttggtgacactctataagattccaaaaagcacatattagtgcagccaagtagcctctcctgtatttaacaaaaagtgcttcattctttgcaggaggcccaacctcctatatataggtttctattcttgatttatttgcttcttcgaaaatctagaggaaaagaaagaagttattttccaggtacccttctcgcttttgccattagccaaggatagaagctgcagtggtattaattttgatataatctttcaaaccagcttgttgtggcttcccttttctttgttcaagatgagggccaggaggggaaacatcacacctgccctaaaccctgttcctggaggtcagcatttgatctgttgcaagcccctctttctgtcccctcttcctaccctgcctcccatgactttgctcctcacacttttggaaccatgccttccgggggggcccatctcttctggcggtccttgtctctgggccacttggagtgtgtgataaatcagtcaagctgttgaagtctcaggagtctctggtagcctgcagaagtaagcctcatcatcagagcctttcctcaaaactggagtcccaaatgtcatcaggttttgttttttttcagccactaagaacccctctgcttttaactctagaatttgggcttggaccagatctaacatcttgaatactctgccctctagagccttcagccttaatggaaggttggatccaaggaggtgtaatggaatcggaatcaagccactcggcaggcatggagctataactaagcatccttagggttctgcctctccaggcattagccctcacattagatctagttactgtggtatggctaatacctgtcaacatttggaggcaatcctaccttgcttttgcttctagagcttagcatatctgattgttgtcaggccatattatcaatgtttacttttttggtactataaaagctttctgccacccctaaactccaggggggacaatatgtgccaatcaatagcacccctactcacatacacacacacctagccagctgtcaagggcagaatgaatctatgctggataagaaatggtggaactgcgttatgaagagctaatttactggacaaagaattccaaagcaaaaccagaacagtatgaatttgagcaggtctcataggttgagcaatttccccctaaaccaactgaaggctaaaaagcaacaggccattgtgaaccaatgcaagacgccctctatcatggtgaaaagctccatcaatgaggtatcttctttagtggtggtatgtaatggaacttagccatttttcaaagcaattgaaatgcattgctctggatctgttccttggcagtggactcagaaagccaacatgtggctcctcccagcccataaccagtatttttgctgcttctgaatacaaattggttggttttgacttcagattgaacttactgtagcctcagatgatttcccccctccgcctcccaggaagaaagaatgttactgccttaataaaaaatgaaaagagaatgatgctcaaaatctttccaaataaaatgttccctat

SEQ ID NO: 55 - DNA, 653 bp, Homo sapiens
  misc_feature (1)..(653): n is a, c, g, or t
ttcccggantttttccntttanttaccaancnntnaaccnaantnccgnnnngaccntaantnncnattntattntannatnggnnangcnatttccccccncaannanganttcaatttnggcnantngtcctgacttangccaaaaatnngttncancnanangaccatatganggnangtaaccnatttattaccttaaaatgtttccccacataagggaaatgngtaagacgagtaaggggaaaagagaaataagagatgtgataaaacctattccaccatctttatcttcatccacagcatgtcctagacattcagtgcctgggccagtctctatagtaaacttggggctgatctggaaacgtctccacttagctctgtaagggatgggcacccttcaggcgtttccaggatgaaactttactggaattcaacccaagtgtcctggaaaggaaattggaagaagttactccaagagctggaactttaaaacaaggagtagctgaagggtgangcatatcccatcctgngttctgagcactctggnaccatgggatgtctgtacactggccntcgccaaagccaagaaagccctggtaggngtcacggcngccatgggtgagctgnaaggcntttccngactccngggg

SEQ ID NO: 56 - DNA, 3846 bp, Homo sapiens
gcctcccgccagctcgcctcggggaacaggacgcgcgtgagctcaggcgtccccgccccagcttttctcggaaccatgaaccccaactgcgcccggtgcggcaagatcgtgtatcccacggagaaggtgaactgtctggataagttctggcataaagcatgcttccattgcgagacctgcaagatgacactgaacatgaagaactacaagggctacgagaagaagccctactgcaacgcacactaccccaagcagtccttcaccatggtggcggacaccccggaaaaccttcgcctcaagcaacagagtgagctccagagtcaggtgcgctacaaggaggagtttgagaagaacaagggcaaaggtttcagcgtagtggcagacacgcccgagctccagagaatcaagaagacccaggaccagatcagtaatataaaataccatgaggagtttgagaagagccgcatgggccctagcgggggcgagggcatggagccagagcgtcgggattcacaggacggcagcagctaccggcggcccctggagcagcagcagcctcaccacatcccgaccagtgccccggtttaccagcagccccagcagcagccggtggcccagtcctatggtggctacaaggagcctgcagccccagtctccatacagcgcagcgccccaggtggtggcgggaagcggtaccgcgcggtgtatgactacagcgccgccgacgaggacgaggtctccttccaggacggggacaccatcgtcaacgtgcagcagatcgacgacggctggatgtacgggacggtggagcgcaccggcgacacggggatgctgccggccaactacgtggaggccatctgaacccggagcgcccccatctgtcttcagcacattccacggcatcgcatccgtcctgggcgtgagccgtccattcttcagtgtctctgttttttaaaacctgcgacagcttgtgattcctacccctcttccagcttcttttgccaactgaagccttcttctgccacttctgcgggctccctcctctggcaggcttcccccgtgatcgacttcttggttttctctctggatggaacgggtatgggcctctctgggggaggcagggctggaatgggagacctgttggcctgtgggcctcacctgcccctctgttctctcccctcacatcctcctgcccagctcctcacatacccacacattccagggctggggtgagcctgactgccaggaccccaggtcaggggctccctacattccccagagtgggatccacttcttggttcctgggatggcgatggggactctgccgctgtgtagggaccagtgggatgggctctacctctctttctcaaagagggggctctgcccacctggggtctctctccctacctccctcctcaggggcaacaacaggagaatggggttcctgctgtggggcgaattcatcccctccccgcgcgttccttcgcacactgtgattttgccctcctgcccacgcagacctgcagcgggcaaagagctcccgaggaagcacagcttgggtcaggttcttgcctttcttaattttagggacagctaccggaaggaggggaacaaggagttctcttccgcagcccctttccccacgcccacccccagtctccagggacccttgcctgcctcctaggctggaagccatggtcccgaagtgtagggcaagggtgcctcaggaccttttggtcttcagcctccctcagcccccaggatctgggttaggtggccgctcctccctgctcctcatgggaagatgtctcagagccttccatgacctcccctccccagcccaatgccaagtggacttggagctgcacaaagtcagcagggaccactaaatctccaagacctggtgtgcggaggcaggagcatgtatgtctgcaggtgtctgacacgcaagtgtgtgagtgtgagtgtgagagatggggcgggggtgtgtctgtaggtgtctctgggcctgtgtgtgggtggggttatgtgagggtatgaagagctgtcttcccctgagagtttcctcagaacccacagtgagaggggagggctcctggggcagagaagttccttaggttttctttggaatgaaattcctccttccccccatctctgagtggaggaagcccaccaatctgccctttgcagtgtgtcagggtggaaggtaagaggttggtgtggagttggggctgccatagggtctgcagcctgctggggctaagcggtggaggaaggctctgtcactccaggcatatgtttccccatctctgtctggggctacagaatagggtggcagaagtgtcaccctgtgggtgtctccctcgggggctcttcccctagacctccccctcacttacataaagctcccttgaagcaagaaagagggtcccagggctgcaaaactggaagcacagcctcggggatggggagggaaagacggtgctatatccagttcctgctctctgctcatgggtggctgtgacaaccctggcctcacttgattcatctctggttttcttgccaccctctgggagtccccatcccattttcatcctgagcccaaccaggccctgccattggcctcttgtcccttggcacacttgtacccacaggtgaggggcaggacctgaaggtattggcctgttcaacaatcagtcatcatgggtgtttttgtcaactgcttgttaattgatttggggatgtttgccccgaatgagaggttgaggaaaagactgtgggtggggaggccctgcctgacccatcccttttcctttctggccccagcctaggtggaggcaagtggaatatcttatattgggcgatttgggggctcggggaggcagagaatctcttgggagtcttgggtggcgctggtgcattctgtttcctcttgatctcaaagcacaatgtggatttggggaccaaaggtcagggacacatccccttagaggacctgagtttgggagagtggtgagtggaagggaggagcagcaagaagcagcctgttttcactcagcttaattctccttcccagataaggcaagccagtcatggaatcttgctgcaggccctccctctactcttcctgtcctaaaaataggggccgttttcttacacacccccagagagaggagggactgtcacactggtgctgagtgaccgggggctgctgggcgtctgttctttaccaaaaccatccatccctagaagagcacagagccctgaggggctgggctgggctgggctgagcccctggtcttctctacagttcacagaggtctttcagctcatttaatcccaggaaagaggcatcaaagctagaatgtgaatataacttttgtgggccaatactaagaataacaagaagcccagtggtgaggaaagtgcgttctcccagcactgcctcctgttttctccctctcatgtccctccagggaaaatgactttattgcttaatttctgcctttcccccctcacacatgcacttttgggcctttttttatagctggaaaaaacaaaataccaccctacaaacctgtatttaaaaagaaacagaaatgaccacgtgaaatttgcctctgtccaaacatttcatccgtgtgtatgtgtatgtgtgtgagtgtgtgaagccgccagttcatctttttatatggggttgttgtctcattttggtctgttttggtcccctccctcgtgggcttgtgctcgggatcaaacctttctggcctgttatgattctgaacatttgacttgaaccacaagtgaatctttctcctggtgactcaaataaaagtataattttta

SEQ ID NO: 57 - DNA, 417 bp, Homo sapiens
gaacctcctccgctgggggacgggaagcccaccgactttgaggatctggaggacggagaggacctgttcaccagcactgtctccaccctagagtcaagtccatcatctccagaaccagctagtcttccatgcagaagatattagtgcaaactccaatggcccaaaacccacagaagttgtattagatgatgacagagaagatctttttgcagaagccacagaagaagtttctttggacagccctgaaaggaacctatcctatcctcggaaccttctcctgcagtcacacctgtcactcctactacactcattgctcctagaattgaatcaaagagtatgtctgctcccgtgatctttgatagatccagggaagagattgaagaagaagcaaatggagacatttttgacatagaaatt

SEQ ID NO: 58 - DNA, 2091 bp, Homo sapiens
gtgacgggtccgcgaggcccagctcgcgcagtcgttcgggtgagcgaagatggcggccgagagggaacctcctccgctgggggacgggaagcccaccgactttgaggatctggaggacggagaggacctgttcaccagcactgtctccaccctagagtcaagtccatcatctccagaaccagctagtcttcctgcagaagatattagtgcaaactccaatggcccaaaacccacagaagttgtattagatgatgacagagaagatctttttgcagaagccacagaagaagtttctttggacagccctgaaagggaacctatcctatcctcggaaccttctcctgcagtcacacctgtcactcctactacactcattgctcctagaattgaatcaaagagtatgtctgctcccgtgatctttgatagatccagggaagagattgaagaagaagcaaatggagacatttttgacatagaaattggtgtatcagatccagaaaaagttggtgatggcatgaatgcctatatggcatatagagtaacaacaaagacatctctttccatgttcagtaagagtgaattttcagtgaaaagaagattcagcgactttcttggtttgcacagcaaattagcaagcaaatatttacatgttggttatattgtgccaccagctccagaaaagagtatagtagggatgaccaaggtcaaagtgggtaaagaagactcatcatccactgagtttgtagaaaaacggagagcagctcttgaaaggtatcttcaaagaacagtaaaacatccaactttactacaggatcctgatttaaggcagttcttggaaagttcagagctgcctagagcagttaatacacaggctctgagtggagcaggaatattgaggatggtgaacaaggctgccgacgctgtcaacaaaatgacaatcaagatgaatgaatcggatgcatggtttgaagaaaagcagcagcaatttgagaatctggatcagcaacttaggaaacttcatgtcagtgttgaagccttggtctgtcatagaaaagaactttcagccaacacagctgcctttgctaaaagtgctgccatgttaggtaattctgaggatcatactgctttatctagagctttgtctcagcttgcagaggttgaggagaagatagaccagttacatcaagaacaagcttttgctgacttttatatgttttcagaactacttagtgactacattcgtcttattgctgcagtgaaaggtgtgtttgaccatcgaatgaagtgctggcagaaatgggaagatgctcaaattactttgctcaaaaaacgtgaagctgaagcaaaaatgatggttgctaacaaaccagataaaatacagcaagctaaaaatgaaataagagagtgggaggcgaaagtgcaacaaggggaaagagattttgaacagatatctaaaacgattcgaaaagaagtgggaagatttgagaaagaacgagtgaaggattttaaaaccgttatcatcaagtacttagaatcactagttcaaacacaacaacagctgataaaatactgggaagcattcctacctgaagccaaagccattgcctagcaataagattgttgccgttaagaagaccttggatgttgttccagttatgctggattccacagtgaaatcatttaaaaccatctaaataaaccactatatattttatgaattacatgtggttttatatacacacacacacacacacacacacacacacacacactctgacattttattacaagctgcatgtcctgaccctctttgaattaagtggactgtggcatgacattctgcaatactttgctgaattgaacactattgtgtcttaaatacttgcactaaatagtgcactgcaagaccagaaaattttacaatattttttctttacaatatgttctgtagtatgtttaccctctttatgaagtgaattaccaatgctttgaataatgttcacttatacattcctgtacagaaattacgattttgtgattacagtaataaaatgatattccttgtgaaaaaaaaaaaaaaaaaaaaaaaaa

SEQ ID NO: 59 - DNA, 415 bp, Homo sapiens
catatttatcgggatgacaaatccatagaatatattcttttatgttaaattatgatcttcatattaatcttaaaattttgtgacgtgtctttttcctttttttccacagttttaatatattattcttcaacgacattttttgtaactttacacttttttggttattttattttaaaaaaatgaaaaattaatttaaaaaaatgcaaaaaactgttggattatttattttagaaattcccccctgttgtgttggactgcaaattgagtttctttctctttaggcctttcacaactaggactgagaatgtatgtaaaagttctgtgacagtacagaaggaaaacaactttttatgtatagcttctaaaaggggaaaaaaaaaaaaagagaaaccctttgacttccacgtgcccatct

SEQ ID NO: 60 - DNA, 3022 bp, Homo sapiens
cggagtcagaaaggcgaggggcgccgggaactggcgtgtgggactccagacaggagaggctgcgccttccccgcaccgggaccttcgcgacacaccagatcctcgcccctggctcgcgcgaacgcacaggatgaccaccaccctcgtgtctgccaccatcttcgacttgagcgaagttttatgcaagggtaacaagatgctcaactatagtgctcccagtgcagggggttgcctgctggacagaaaggcagtgggcacccctgctggtgggggcttccctcggaggcactcagtcaccctgcccagctccaagttccaccagaaccagctcctcagcagcctcaagggtgagccagcccccgctctgagctcgcgagacagccgcttccgagaccgctccttctcggaagggggcgagcggctgctgcccacccagaagcagcccgggggcggccaggtcaactccagccgctacaagacggagctgtgccgcccctttgaggaaaacggtgcctgtaagtacggggacaagtgccagttcgcacacggcatccacgagctccgcagcctgacccgccaccccaagtacaagacggagctgtgccgcaccttccacaccatcggcttttgcccctacgggccccgctgccacttcatccacaacgctgaagagcgccgtgccctggccggggcccgggacctctccgctgaccgtccccgcctccagcatagctttagctttgctgggtttcccagtgccgctgccaccgccgctgccaccgggctgctggacagccccacgtccatcaccccaccccctattctgagcgccgatgacctcctgggctcacctaccctgcccgatggcaccaataacccttttgccttctccagccaggagctggcaagcctctttgcccctagcatggggctgcccgggggtggctccccgaccaccttcctcttccggcccatgtccgagtcccctcacatgtttgactctccccccagccctcaggattctctctcggaccaggagggctacctgagcagctccagcagcagccacagtggctcagactccccgaccttggacaactcaagacgcctgcccatcttcagcagactttccatctcagatgactaagccagggtagggagggacctcctgcctactccagcccctaccctgcacccacatcccataccctcttctccctacccatcccattccccacaggccctacattaacaaggttaagctcaacccctttcccccagcacctcagaatgtgccctccctctccccctcataaccccacctaacataaggacaagtcaatttgtcagtagcttcttctggcttgaaaccccctccctggattttatagcccacttaccatgcataacagacaagtcccatattttgtcagtagatgcctttttttttccggcttaagccttaagtgccaaatcacaagagaaaaagcagtaacagtttacagaagcaacttagtgccttgtaatctaactttgtcactgtgactacattacctcttcagcgccagagggcacccgtgggcctcccggagcctctgcccatggcggggtggagacccggaaccagcagccccctccactggcgacacaactgcaccttccctcatttcagtctcccgcacacttattcctcctcccctcttcccggtggcacctctccacctgtacccgcccccccaccccccccacccctgccccttggaagagttgttgccagaccagggttttgggggaaacctgtcttgacattcaaaacctttttcttcccgatctgaacccctgttgactaatcttgcctgggtttgtgtaggtctgcaggaaggaaggctgaaaaagcggacgaagattttgacttaagtgggactttgtgatttaattttttcttttttttaagtggggaggaaggggaagctagatggactaggagagacttgattttggtgctaaagttccccagttcatatgtgacatctttttaaaaaaaataacaacaaaaaaaaaatgagagaaaagctaaaaaaaaaaaagtaaggggtgagcagttaatggtattcattccacatacaatatctgtgtaaaacgatttcctgtagaagtagctttaatggtttttgctctagaataccgtaggtctatccttagagcactcacgccatgctttcttccctgggttttaaacttcatataactttcagaaattggagagcaaaaattttgcttgtcactgcacatcaatataaaaaagcttatttaacttatcaaaacgtatttattgccaaactatgcttttttttgttaattttgttcatatttatcgggatgacaaatccatagaatatattcttttatgttaaattatgatcttcatattaatcttaaaattttgtgacgtgtctttttcctttttttccacagttttaatatattattcttcaacgacattttttgtaactttacacttttttggttattttattttaaaaaaatgaaaaattaatttaaaaaaatgcaaaaaactgttggattatttattttagaaattcccccctttgtgttggactgcaaattgagtttctttctctttaggcctttcacaactaggactgagaatgtatgtaaaagttctgtgacagtacagaaggaaaacaactttttatgtatagcttctaaaaggggaaaaaaaaaaaaagagaaaccctttgacttccacgtgcccatctcaagacattccactcacagatttgaggttctggattccaggtctggagttttccaatgttaatgtaaacagaactggcacacacacattaagatgaatgtaattattattcctcttgctggtcactaccgtcgctttctatttctctttctttgtgtgaatttatttaaaagaaaaaaaaactttttgtaacgactatttgcagtttaaaaatcaataaaccccgttttttcaagaaacatt

SEQ ID NO: 61 - DNA, 606 bp, Homo sapiens
tttaggatttttagtattttaatataatacagttccaatcagtgtgtctggttcatttcagttccttcactgccaaaccggggggcagggactgcttcagtttctctgccttctctttgtcagtgatgaccagggtgtaaaggtatctgctgcatcgaactttaaacttcacgttgtccttatttttcttgatcttgacagatttggcatcctttcgtcgggctgtgagcttttctacaaaaatggtgatttatttaacatacaatggtagatatattggggatcatcaaatttttaaaaatttttgttgggcttactttacatattacaatactaacgtaagcttataaactcttccatttcagtgaaggaaagggatccaattaacacctgctagcagcttggctaagaacggacattaaaatcagacaaaacactggaaatataaagtacttttctccaaatgtgtataccaagttttggaaccaaatcactgcctgctccacaggacagaccaattgaattttcaaaaatgcttcaggtcgggggattgcactaccactgtgtaacaatacttattataccgctcacaatatggtatcgaag

SEQ ID NO: 62 - DNA, 5075 bp, Homo sapiens
tgaattcggcacgaggtgggctctctcttttcccctcttgctcctttcttttctttttttctgtttttttaaaccttccaaggcaagttcatggatactaagctgatgtgtttgttgttctttttctccctgcctccgctcctagtgagtaaccacactggccgcatcaaggtggtctttactccgagcatctgtaaagtgacctgcaccaagggcagctgtcagaacagctgtgagaaggggaacaccaccactctcattagtgagaatggtcatgctgccgacaccctgacggccacgaacttccgagtggtaatttgccatcttccatgtatgaatggtggccagtgcagttcaagggacaaatgtcagtgccctccaaatttcacaggaaaactttgtcagatcccagtccatggtgccagcgtgcctaaactttatcagcattcccagcagccaggcaaggcgttggggacgcatgtcatccattcaacacataccttgcctctgaccgtgactagccagcaaggagtcaaagtgaaatttcctcctaacatagtcaatatccatgtgaaacatcctcctgaagcttccgtccagatacatcaggtttcaagaattgatggcccaacaggccagaagacaaaagaagctcaaccaggccaatcccaagtctcgtaccaagggcttcctgtccagaagacccagaccatacattccacatactcccaccagcaggtcattcctcacgtctaccccgtggctgctaagacacagcttggccggtgcttccaggaaaccattgggtcacagtgtggcaaagcgctccctggcctttcaaagcaagaggactgctgtggaactgtgggtacctcctggggctttaacaaatgccagaaatgccccaagaaaccatcttatcatggatacaaccaaatgatggaatgcctaccgggttataagcgggttaacaacaccttttgccaagatattaatgaatgtcagctacaaggtgtatgccctaatggtgagtgtttgaataccatgggcagctatcgatgtacctgcaaaataggatttgggccggatcctaccttttcaagttgtgttcctgatccccctgtgatctcggaagagaaagggccctgttaccgacttgtcagttctggaagacagtgtatgtaccctctgtctgttcacctcaccaagcagctctgctgttgtagtgtgggcaaggctgggccacactgtgagaaatgtccccttccaggcacagctgcttttaaggaaatctgtcctggtggaatgggttatacggtttctggcgttcatagacgcaggccaatccatcaccatgtaggtaaaggacctgtatttgtcaagccaaagaacactcaacctgttgctaaaagtactcatcctccacctctcccagccaaggaagagccagtggaggccctgaccttctcccgggaacacggggccaggagtgcggagccagaagtggcaactgcaccccctgaaaaggaaataccttcattggatcaagagaaaaccaaacttgagcctggtcaaccccagctgtctccaggcatttccgctattcatctgcatccacagtttccagtagtgattgaaaaaacatcacctcctgtgcctgttgaagtagctcctgaagcttctacgtctagtgccagccaagtgattgctcctactcaagtgacagaaatcaatgaatgtactgtgaaccctgatatctgtggagcaggacactgcattaacctaccagtgagatatacctgtatatgctacgagggctacaggttcagtgaacaacagaggaaatgtgtggatattgatgagtgtactcaggtccaacacctctgctcccagggccgctgtgaaaacaccgagggaagtttcttgtgcatttgcccagcaggatttatggccagtgaggagggtactaactgcatagatgttgacgaatgcctgaggccggacgtctgtggggaggggcactgtgtcaatactgtgggggccttccggtgtgaatactgtgacagcgggtaccgcatgactcagagaggccgttgtgaggatattgatgaatgtttgaatccaagcacttgtccagatgagcagtgtgtgaattctcctggatcttaccagtgcgttccctgcacagaaggattccgaggctggaatggacagtgccttgatgtggacgagtgcctggaaccaaacgtctgcgcaaatggtgattgttccaaccttgaaggctcctacatgtgttcatgccacaaaggctatacccggactccggaccacaagcactgtagagatattgatgaatgtcagcaagggaatctatgtgtaaacgggcagtgcaaaaataccgagggctccttcaggtgcacctgtggacaggggtaccagctgtcggcagctaaagaccagtgtgaagacattgatgaatgccagcaccgtcatctctgtgctcatgggcagtgcaggaacactgagggctcttttcaatgtgtgtgtgaccagggttacagagcatctgggcttggagaccactgtgaagatatcaatgaatgcttggaggacaagagtgtttgccagagaggagactgcattaatactgcagggtcctatgattgtacttgtccggatggatttcagctagatgacaataaaacatgtcaagatattaatgaatgtgaacatccagggctctgtggtccgcaaggggagtgcctaaacacagagggttctttccattgtgtctgccagcagggtttctcaatctctgcagatggccgtacgtgtgaagatattgatgaatgtgtaaacaacactgtttgtgacagtcacgggttttgtgacaatacagctggctccttccgctgcctctgttatcagggctttcaagccccacaggatgggcaagggtgtgtggatgtgaatgaatgtgaactgctcagtggggtgtgtggtgaagccttctgtgaaaacgtggaagggtccttcctgtgcgtgtgtgctgatgaaaaccaagagtacagccccatgactgggcagtgccgctcccggacctccacagatttagatgtagatgtagatcaacccaaagaagaaaagaaagaatgctactataatctcaatgacgccagtctctgtgataatgtgttggcccccaatgtcacgaaacaagaatgctgctgtacatcaggcgcgggatggggagataactgcgaaatcttcccctgcccggtcttgggaactgctgagttcactgaaatgtgtcccaaagggaaaggttttgtgcctgctggagaatcatcttctgaagctggtggtgagaactataaagatgcagatgaatgcctactttttggacaagaaatctgcaaaaatggtttctgtttgaacactcggcctgggtatgaatgctactgtaagcaagggacgtactatgatcctgtgaaactgcagtgctttgatatggatgaatgtcaagaccccagtagttgtattgatggccagtgtgttaatacagagggctcttacaactgcttctgtactcaccccatggtcctggatgcgtcagaaaaaagatgtatacgaccggctgagtcaaacgaacaaatagaagaaactgatgtctaccaagatttgtgctgggaacatctgagtgatgaatacgtgtgtagccggcctcttgtgggcaagcagacaacgtacactgagtgctgctgtctgtatggagaggcctggggcatgcagtgtgccctctgccccctgaaggattcagatgactatgctcagctgtgtaacatccccgtgacgggacgccggcagccatatggacgggacgccttggttgacttcagtgaacagtatactccagaagccgatccctacttcatccaagaccgttttctaaatagctttgaggagttacaggctgaggaatgcggcatcctcaatggatgtgaaaatggtcgctgtgtgagggtccaggaaggttacacctgcgattgcttggatgggtatcacttggatactgccaagatgacctgtttcgatgtaaatgaatgcgatgagttgaacaaccggatgtctctctgcaagaatgccaagtgcattaacaccgatggttcctacaagtgtttgtgtctgccaggctacgtgccttctgacaagccaaactactgcactccgttgaataccgccttgaatttagagaaagacagtgacctggagtgaaacagaatctacataacctaagcccatatactctgcactgtgtaaaggaaaagggagaaatgtattatacttgagacattgcacctaccccggaaggctggaaatacagaaacagcatggagttgcaagtcctctgaagacaatgagaggatttaggatgagcccgataggtgtggcagaccaaatggacatttctctaaaaaaccagtatatatagtctgttcatatgtaaaattcaatggaagagaggtggaacagtgctgttattttaaacagaaggttgtattattatgttgttttgttttttttactattgcttgattaaatttggcatttaaatagtggtggaaatatttttatataattttcattttttggttgtgcagttccttggctactgtttttcttttacttcagttttttaaaaatctcaaatgaaaaagtcttcgatacaatattgttaagctgtattataagtattgttacacagggttatgcaattcccggcctggagcatttttgaaattcaaattgtctgtcctgtggagcaggcagtgattttgttccaaaactttgtatacacatttggagaaaagtactttatattttcagtgttttgtctgattttaatgtccgttcttagccaagctgctagcaggtgttaattggatccctttccttcactgaaatggaagagtttataagcttacgttagtattgtaatatgtaaagtaagcccaacaaaaatttttaaaaatttgatgatccccaatatatctaccattgtatgttaaataaat

SEQ ID NO: 63 - DNA, 565 bp, Homo sapiens
  misc_feature (1)..(565): n is a, c, g, or t
aaagaagtaaaatataaatttttatttagaattaaagcaaatacttcagtatcacaaacacaatattaaacttcaagaaatacaccgcgaattcggagtgagcgtttatttgcacgattacaacatgcggacaaacccaggcgagatgagcgagtctacggttcctacccacccggctgacaaggggtggccccaggggcggagtgcccagccccgaggcggccctactcgtcctcctctcccgaagactctctctcgaacgtgtaggccatgaagcaagcgtcgggtctcttggggacaaggggatgccccggtctcacaatctcaaagcccaaaaagctgaaggttcggagcaaggcggctctgtcctcgcggttcttgtggaagcaaatgaagacatggtcggctcgcagctgctcctcagcgaactccaggagaactgcaaagctgtccttgctcccctcgggcagcgcgccgcccgggatctcgatgtaganggtgccgccactcagcactgttcgccgttaatgcggttggcgtctgttagcctggactggaggttgag

SEQ ID NO: 64 - DNA, 986 bp, Homo sapiens
tgcgaacggcgagcagcggcggcggcgcggagagacgcagcggaggttttcctggtttcggaccccagcggccggatggtgaaatcctccctgcagcggatcctcaatagccactgcttcgccagagagaaggaaggggataaacccagcgccaccatccacgccagccgcaccatgccgctccttagcctgcacagccgcggcggcagcagcagtgagagttccagggtctccctccactgctgtagtaacccgggtccggggcctcggtggtgctcctgatgcccctcacccacccctgaagatcccaggtgggcgagggaatagtcagagggatcacaatctttcagctaacttattctactccgatgatcggctgaatgtaacagaggaactaacgtccaacgacaagacgaggattctcaacgtccagtccaggctcacagacgccaaacgcattaactggcgaacagtgctgagtggcggcagcctctacatcgagatcccgggcggcgcgctgcccgaggggagcaaggacagctttgcagttctcctggagttcgctgaggagcagctgcgagccgaccatgtcttcatttgcttccacaagaaccgcgaggacagagccgccttgctccgaaccttcagctttttgggctttgagattgtgagaccggggcatccccttgtccccaagagacccgacgcttgcttcatggcctacacgttcgagagagagtcttcgggagaggaggaggagtagggccgcctcggggctgggcatccggcccctggggccaccccttgtcagccgggtgggtaggaaccgtagactcgctcatctcgcctgggtttgtccgcatgttgtaatcgtgcaaataaacgctcactccgaattagcggtgtatttcttgaagtttaatattgtgtttgtgatactgaagtatttgctttaattctaaataaaaatttatattttacttttt

SEQ ID NO: 65 - DNA, 173 bp, Homo sapiens
acctagatatgtacatatttattacaatataaagacataaatgtatatttctgaaatattctaaaaatataatgtaggaagaaatatttatcaaatacacagaattatatacagtatgcatttaggacaagcagtagtaaaaatatgcacaaataaaccatctgtccacacaa

SEQ ID NO: 66 - DNA, 3265 bp, Homo sapiens
tggagagggacaggaggaaacgcagcgccagcagcatctcatctaccctccttgacacctccccgtggctccagcaagccctagaggtcagccttgcggaccaacaggaggactcccagctttcccttttcaagaggtaccccagacaccggccaccctcttccagcccctgcggccagtgcaaggaggcaccaatgctctgaggctgtcgcgtggtgcagcgtcgagcatcctcgccgagtccttctgctgcctgtcccgcctcaccccgctccatcacaccagctggccctctttgcttccttttcccagaatcgttaagccccgactcccactagcacctcgtaccaacctcgccccaccccatcctcctgccttcccgcgctccggtgtcccccgctgccatgagctcccccatcagcaagagccgctcgcttgccgccttcctgcagcagctgcgcagtccgaggcagcccccgagactggtgacatctacggcgtacacgtcccctcagccgcgagaggtgccagtctgcccgctgacagctggtggcgagactcagaacgcggccgccctgccgggccccaccagctggccactgctggcgagcctgctgcagattctctggaaagggggtctcaagaaacagcacgacaccctggtggagtaccacaagaagtatggcaagattttccgcatgaagttgggttcctttgagtcggtgcacctgggctcgccatgcctgctggaagcgctgtaccgcaccgagagcgtaccccagcggctggagatcaaaccgtggaaggcctatcgcgactaccgcaaagaaggctacgggctgctgatcctggaaggggaagactggcagcgggtccggagtgcctttcaaaagaaactaatgaaaccaggggaagtgatgaagctggacaacaaaatcaatgaggtcttggccgattttatgggcagaatagatgagctctgtgatgaaagaggccacgtcgaagacttgtacagcgaactgaacaaatggtcgtttgaaagtatctgcctcgtgttgtatgagaagagatttgggcttctccagaagaatgcaggggatgaagctgtgaacttcatcatggccatcaaaacaatgatgagcacgtttgggaggatgatggtcactccagtcgagctgcacaagagcctcaacaccaaggtctggcagggacacactctggcctgggacaccattttcaaatcagtcaaagcttgtatcgacaaccggttagagaagtattctcagcagcctagtgcagatttcctttgtgacatttatcaccagaatcggctttcaaagaaagaattgtatgctgctgtcacagagctccagctggctgcggtggaaacgacagcaaacagtctaatgtggattctctacaatttatcccgtaatccccaagtgcaacaaaagcttcttaaggaaattcaaagtgtattacctgagaatcagaggccacgggaggaagatttgaggaatatgccgtatttaaaagcctgtctgaaagaatctatgaggcttaccccgggtgtaccatttacaactcggactcttgacaaggcaacagttctgggtgaatatgctttacccaaaggaacagtgctcatgctaaatacccaggtgttgggatccagtgaagacaattttgaagattcaagtcagtttagacctgaacgttggcttcaggagaaggaaaaaattaatccttttgcgcatcttccatttggcgttggaaaaagaatgtgcattggtcgccgattagcagagcttcaactgcatttggctctttgttggattgtccgcaaatacgacatccaggccacagacaatgagcctgttgagatgctacactcaggcaccctggtgcccagccgggaactccccatcgcgttttgccagcgataatacgcctcagatggtggtatttgctaacatcatatccaactcagggaagcggactgagtgctgggatccaaggcattctacagggttcactgctggtttacacttcacctgtgtcagcaccatcttcaggtgcttagaatggcctgggagcctgttctgtcttgcatcttccatgacatgaaagggaggctggcacttgtcagtcaggtagaggttacaaaccgtttcaggccctgctaccacattcactgtttgaatctttaattcccaagaataagtttacatttcacaatgaatgacctacaacagctaaattttctggggctgggagtaatactgacaatccatttactgtagctctgcttaatgtactacttaggaaaatgtccctgcttaataatgtaagccaagctaaatgatggttaaagttatcaggcctcccatgaaattgcgttcttcctgcattgaaataaaaacattattgggaaactagagaacacctctatttttaaaaggactttaacgaagtcaaacaacttctaagactagtgattcactggggcattatttgttagaggaccttaaaattgtttattttttaaatgtgattcctttatggcattagggtaaagatgaagcaataatttttaaattgtgtatgtgcatatgaagcacagacatgcatgtgtgtgtgtgtctgtgtgtgtgtgtccgtgtatgtgtgtgtgggttctaatggtaatttgcctcagtcatttttttaatatttgcagtacttgatttaggatctgtggtgcagggcatgtttcaaagtttagtcacagcttaaaaacattcagtgtgactttaatattataaaatgatttcccatgccataatttttctgtctattaaatgggacaagtgtaaagcatgcaaaagttagagatctgttatataacatttgttttgtgatttgaactcctaggaaaaatatgatttcataaatgtaaaatgcacagaaatgcatgcaatacttataagacttaaaaattgtgtttacagatggtttatttgtgcatatttttactactgcttttcctaaatgcatactgtatataattctgtgtatttgataaatatttcttcctacattatatttttagaatatttcagaaatatacatttatgtctttatattgtaataaatatgtacatatctaggtatatgctttctctctgctgtgaaattatttttagaattataattcacgtcttgtcatatttcatctgtataccttcaaattctctgaaagtaaaaataaaagtttttaaatatt

SEQ ID NO: 67 - DNA, 337 bp, Homo sapiens
  misc_feature (1)..(337): n is a, c, g, or t
gaggttatttaaaattatttacactcactggaaatcatgtggaggggctgggcatgcttcagcagagagaaagagcccttctgtgcagccaccgagggccagacnccatgaggcatggagcccggtgcccccatgcaggccctggntgtgctgccagggncggtggacctcacctggcctggggntgaggaggntgcaccccaggcccagaggagtntttntcactgggcctgcgccattgccaaggccaagccaaacacacccaggagantacgggagggagccaggagagctgattacaggccggattctgtgggttnggtnggncaggcaggcc

SEQ ID NO: 68 - DNA, 2446 bp, Homo sapiens
ggctgccagcgtttcatgatcaacatgggagactcccacgtggacaccagctccaccgtgtccgaggcggtggccgaagaagtatctcttttcagcatgacggacatgattctgttttcgctcatcgtgggtctcctaacctactggttcctcttcagaaagaaaaaagaagaagtccccgagttcaccaaaattcagacattgacctcctctgtcagagagagcagctttgtggaaaagatgaagaaaacggggaggaacatcatcgtgttctacggctcccagacggggactgcagaggagtttgccaaccgcctgtccaaggacgcccaccgctacgggatgcgaggcatgtcagcggaccctgaggagtatgacctggccgacctgagcagcctgccagagatcgacaacgccctggtggttttctgcatggccacctacggtgagggagaccccaccgacaatgcccaggacttctacgactggctgcaggagacagacgtggatctctctggggtcaagttcgcggtgtttggtcttgggaacaagacctacgagcacttcaatgccatgggcaagtacgtggacaagcggctggagcagctcggcgcccagcgcatctttgagctggggttgggcgacgacgatgggaacttggaggaggacttcatcacctggcgagagcagttctggctggccgtgtgtgaacactttggggtggaagccactggcgaggagtccagcattcgccagtacgagcttgtggtccacaccgacatagatgcggccaaggtgtacatgggggagatgggccggctgaagagctacgagaaccagaagcccccctttgatgccaagaatccgttcctggctgcagtcaccaccaaccggaagctgaaccagggaaccgagcgccacctcatgcacctggaattggacatctcggactccaaaatcaggtatgaatctggggaccacgtggctgtgtacccagccaacgactctgctctcgtcaaccagctgggcaaaatcctgggtgccgacctggacgtcgtcatgtccctgaacaacctggatgaggagtccaacaagaagcacccattcccgtgccctacgtcctaccgcacggccctcacctactacctggacatcaccaacccgccgcgtaccaacgtgctgtacgagctggcgcagtacgcctcggagccctcggagcaggagctgctgcgcaagatggcctcctcctccggcgagggcaaggagctgtacctgagctgggtggtggaggcccggaggcacatcctggccatcctgcaggactgcccgtccctgcggccccccatcgaccacctgtgtgagctgctgccgcgcctgcaggcccgctactactccatcgcctcatcctccaaggtccaccccaactctgtgcacatctgtgcggtggttgtggagtacgagaccaaggccggccgcatcaacaagggcgtggccaccaactggctgcgggccaaggagcctgtcggggagaacggcggccgtgcgctggtgcccatgttcgtgcgcaagtcccagttccgcctgcccttcaaggccaccacgcctgtcatcatggtgggccccggcaccggggtggcacccttcataggcttcatccaggagcgggcctggctgcgacagcagggcaaggaggtgggggagacgctgctgtactacggctgccgccgctcggatgaggactacctgtaccgggaggagctggcgcagttccacagggacggtgcgctcacccagctcaacgtggccttctcccgggagcagtcccacaaggtctacgtccagcacctgctaaagcaagaccgagagcacctgtggaagttgatcgaaggcggtgcccacatctacgtctgtggggatgcacggaacatggccagggatgtgcagaacaccttctacgacatcgtggctgagctcggggccatggagcacgcgcaggcggtggactacatcaagaaactgatgaccaagggccgctactccctggacgtgtggagctaggggcctgcctgccccacccaccccacagactccggcctgtaatcagctctcctggctccctcccgtagtctcctgggtgtgtttggcttggccttggcatgggcgcaggcccagtgacaaagactcctctgggcctggggtgcatcctcctcagcccccaggccaggtgaggtccaccggcccctggcagcacagcccagggcctgcatgggggcaccgggctccatgcctctggaggcctctggccctcggtggctgcacagaagggctctttctctctgctgagctgggcccagcccctccacatgatttccagtgagtgtaaataattttaaataacctctggcccttggaataaagttctgttttctgtaaaaaaaaaaaaaaa

SEQ ID NO: 69 - DNA, 154 bp, Homo sapiens
tttttttttttttttaagctcttgtactttattgagcagctgtggaattaggaaggggaagtaggataggggacacgattacgttcctaagtgaaggtttcaagctgggctttgcagggggtataattagtatggtcactgtgtcttgtaggat

SEQ ID NO: 70 - DNA, 1925 bp, Homo sapiens
agtggagtgggacaggtatataaaggaagtacagggcctggggaagaggccctgtctaggtagctggcaccaggagccgtgggcaagggaagaggccacaccctgccctgctctgctgcagccagaatgggtgtgaaggcgtctcaaacaggctttgtggtcctggtgctgctccagtgctgctctgcatacaaactggtctgctactacaccagctggtcccagtaccgggaaggcgatgggagctgcttcccagatgcccttgaccgcttcctctgtacccacatcatctacagctttgccaatataagcaacgatcacatcgacacctgggagtggaatgatgtgacgctctacggcatgctcaacacactcaagaacaggaaccccaacctgaagactctcttgtctgtcggaggatggaactttgggtctcaaagattttccaagatagcctccaacacccagagtcgccggactttcatcaagtcagtaccgccattcctgcgcacccatggctttgatgggctggaccttgcctggctctaccctggacggagagacaaacagcattttaccaccctaatcaaggaaatgaaggccgaatttataaaggaagcccagccagggaaaaagcagctcctgctcagcgcagcactgtctgcggggaaggtcaccattgacagcagctatgacattgccaagatatcccaacacctggatttcattagcatcatgacctacgattttcatggagcctggcgtgggaccacaggccatcacagtcccctgttccgaggtcaggaggatgcaagtcctgacagattcagcaacactgactatgctgtggggtacatgttgaggctgggggctcctgccagtaagctggtgatgggcatccccaccttcgggaggagcttcactctggcttcttctgagactggtgttggagccccaatctcaggaccgggaattccaggccggttcaccaaggaggcagggacccttgcctactatgagatctgtgacttcctccgcggagccacagtccatagaaccctcggccagcaggtcccctatgccaccaagggcaaccagtgggtaggatacgacgaccaggaaagcgtcaaaagcaaggtgcagtacctgaaggataggcagctggcaggcgccatggtatgggccctggacctggatgacttccagggctccttctgcggccaggatctgcgcttccctctcaccaatgccatcaaggatgcactcgctgcaacgtagccctctgttctgcacacagcacgggggccaaggatgccccgtccccctctggctccagctggccgggagcctgatcacctgccctgctgagtcccaggctgagcctcagtctccctcccttggggcctatgcagaggtccacaacacacagatttgagctcagccctggtgggcagagaggtagggatggggctgtggggatagtgaggcatcgcaatgtaagactcgggattagtacacacttgttgatgattaatggaaatgtttacagatccccaagcctggcaagggaatttcttcaactccctgccccctagccctccttatcaaaggacaccattttggcaagctctatcaccaaggagccaaacatcctacaagacacagtgaccatactaattataccccctgcaaagccagcttgaaaccttcacttaggaacgtaatcgtgtcccctatcctacttccccttcctaattccacagctgctcaataaagtacaagagtttaacagtgtgttggcgctttgctttggtctatctttgagcgcccactagacccactggactcacctcccccatctcttctgggttccttcctctgagccttgggacccctgagcttgcagagatgaaggccgccatgtt

SEQ ID NO: 71 - DNA, 513 bp, Homo sapiens
ttttaaaataatgactcattttatttttttcttttaatacgtagttataaatatattttgtcaaaatatatgatcaatatggtcaaaacatttgcacgtaaagtcataaataaggtcaaggtgaatgtttagggattttttccttttttttttttttttttaaataaaagtccagctataaggaagcagtctgtgtagtgtgtgggtgagtggatgggaacgtgtgtgtgtggtgtatgtggtgtgtgtgtgtggtgtgtatatctgtctatcctcaaggactgcctgatctcagcggcacccacatccctctactaccatctcctgataatttttcagtaccaccttatcatactcatccaggtacagcatggagatggcactcagttcagtgggcacacaacaggctttggggatactggaattgacagaattgaccagggtctgcacaatggcatggttggttgagttgaggtggtcaccagtggaaaggggcagtcccatggcagtaga

SEQ ID NO: 72 - DNA, 1999 bp, Homo sapiens
gagggaggggccgccggggaagaggaggaggaaggaaagaaagaaagcgagggagggaaagaggaggaaggaagatgcgagaaggcagaggaggagggagggagggaaggagcgcggagcccggcccggaagctaggtgagtgtggcatccgagctgagggacgcgagcctgagacgccgctgctgctccggctgagtatctagcttgtctccccgatgggattcccgtccaagctatctcgagcctgcagcgccacagtccccggccctcgcccaggttcactgcaaccgttcagaggtccccaggagctgctgctggcgagcccgctactgcagggacctatggagccattccgtagtgccatcccgagcaacgcactgctgcagcttccctgagcctttccagcaagtttgttcaagattggctgtcaagaatcatggactgttattatatgccttgttttctgtcaagacaccatgattcctggtaaccgaatgctgatggtcgttttattatgccaagtcctgctaggaggcgcgagccatgctagtttgatacctgagacggggaagaaaaaagtcgccgagattcagggccacgcgggaggacgccgctcagggcagagccatgagctcctgcgggacttcgaggcgacacttctgcagatgtttgggctgcgccgccgcccgcagcctagcaagagtgccgtcattccggactacatgcgggatctttaccggcttcagtctggggaggaggaggaagagcagatccacagcactggtcttgagtatcctgagcgcccggccagccgggccaacaccgtgaggagcttccaccacgaagaacatctggagaacatcccagggaccagtgaaaactctgcttttcgtttcctctttaacctcagcagcatccctgagaacgaggcgatctcctctgcagagcttcggctcttccgggagcaggtggaccagggccctgattgggaaaggggcttccaccgtataaacatttatgaggttatgaagcccccagcagaagtggtgcctgggcacctcatcacacgactactggacacgagactggtccaccacaatgtgacacggtgggaaacttttgatgtgagccctgcggtccttcgctggacccgggagaagcagccaaactatgggctagccattgaggtgactcacctccatcagactcggacccaccagggccagcatgtcaggattagccgatcgttacctcaagggagtgggaattgggcccagctccggcccctcctggtcacctttggccatgatggccggggccatgccttgacccgacgccggagggccaagcgtagccctaagcatcactcacagcgggccaggaagaagaataagaactgccggcgccactcgctctatgtggacttcagcgatgtgggctggaatgactggattgtggccccaccaggctaccaggccttctactgccatggggactgcccctttccactggctgaccacctcaactcaaccaaccatgccattgtgcagaccctggtcaattctgtcaattccagtatccccaaagcctgttgtgtgcccactgaactgagtgccatctccatgctgtacctggatgagtatgataaggtggtactgaaaaattatcaggagatggtagtagagggatgtgggtgccgctgagatcaggcagtccttgaggatagacagatatacacaccacacacacacaccacatacaccacacacacacgttcccatccactcacccacacactacacagactgcttccttatagctggacttttatttaaaaaaaaaaaaaaaaaaatggaaaaaatccctaaacattcaccttgaccttatttatgactttacgtgcaaatgttttgaccatattgatcatatattttgacaaaatatatttataactacgtattaaaagaaaaaaataaaatgagtcattattttaaaggt

SEQ ID NO: 73 - DNA, 452 bp, Homo sapiens
  misc_feature (1)..(449): n is a, c, g, or t
cctctcatttagtctcagagcctaaaaagaaatgcctccagtctctgttagccccgatgtttggaatgaaattaacaagattctaccttaaaagagaaaacttatgtgggcttttcaaattgtgaaaatttgttccctcttataaaatataatctttcccctgctgctggtttataaatatgggcatatagcccagagccattataaagaaaaaacaaccaaccacaataaactaaggagcctcatctgatggctaaggttctcaggaaaaaatgagaagagcaccttgatcaggaaaggaaaaaaggatagaggataaaaacagtaacctttaacttctatgccctcactcagccagtaacttacggcaggtgttgctggcaaggccttnagctgtatttcttcattaagttgaacatcctgtctgccaactccataccaacccagctgct

SEQ ID NO: 74 - DNA, 2753 bp, Homo sapiens
ctttaccggagaagaggcaaagtacgctcaaagccgaagccacagctcctcctgccgcatttctttcctgcttgcgaattccaagctgttaaataagatgtgcaaagggcttgcaggtctgccggcttcttgcttgaggagtgcaaaagatatgaaacatcggctaggtttcctgctgcaaaaatctgattcctgtgaacacaattcttcccacaacaagaaggacaaagtggttatttgccagagagtgagccaagaggaagtcaagaaatgggctgaatcactggaaaacctgattagtcatgaatgtgggctggcagctttcaaagctttcttgaagtctgaatatagtgaggagaatattgacttctggatcagctgtgaagagtacaagaaaatcaaatcaccatctaaactaagtcccaaggccaaaaagatctataatgaattcatctcagtccaggcaaccaaagaggtgaacctggattcttgcaccagggaagagacaagccggaacatgctagagcctacaataacctgctttgatgaggcccagaagaagattttcaacctgatggagaaggattcctaccgccgcttcctcaagtctcgattctatcttgatttggtcaacccgtccagctgtggggcagaaaagcagaaaggagccaagagttcagcagactgtgcttccctggtccctcagtgtgcctaattctcacctgaaggcagagggatgaaatgccaagactctatgctctggaaaacctgaggccaaatattgatctgtattaagctccagtgctttatccacattgtagcctaatattcatgctgcctgccatgtgtgagtcacttctacgcataaactagatatagcttttggtgtttgagtgttcatcagggtgggaccccattccagtccaattttcctaagtttctttgagggttccatgggagcaaatatctaaataatggcctggtaggtctggattttcaaagattgttggcagtttcctcctcccaacagttttacctcgggatggttggttagtgcatgtcacatgacatccacatgcacatgtattctgttggccagcacgttctccagactctagatgtttagatgaggttgagctatgatatgtgcttgtgtgtatgtctatgtgtatatattatatatacattagacacacatatacattatttctgtatatagatgtctgtgtatacatatgtatgtgtgagtgtatgtatacacacacacacacacacacacacttttgcaagagtgatgggaaagaccctaggtgctcataactagagtatgtgtatgtacttacatgggtgttttgatctctgttctttcatactacatttgaacagggcaaaatgaactaactgccatgtaggctaagaaagaaatgctaacctgtggaaagttggttttgtaaaattccatggatcttgctggagaagcatccaaggaacttcatgcttgatttgaccactgacagcctccaccttgagcactattctaaggagcaaataccttagctcccttgagctggttttctctgatggcacttttgagctcctaagctgccagccttcccttcttttcctgggtgctcagggcatgcttattagcagctgggttggtatggagttggcagacaggatgttcaacttaatgaagaaatacagctaaggccttgccagcaacacctgccgtaagttactggctgagtgagggcatagaagttaaaggttactgtttttatcctctatccttttttcctttcctgatcaaggtgctcttctcattttttcctgagaaccttagccatcagatgaggctccttagtttattgtggttggttgttttttctttataatggctctgggctatatgcccatatttataaaccagcagcaggggaaagattatattttataagagggaacaaattttcacaatttgaaaagcccacataagttttctcttttaaggtagaatcttgttaatttcattccaaacatcggggctaacagagactggaggcatttctttttaggctctgagactaaatgagaggaaaagaaaagaaaaaaaaatgattgtctaaccaattgtgagaattactgtttgaaacttttcaaggcacattgaaatacttgaaaacttctcatttatgttatttatgatgttattttgtacgtgttattattattatattgttttataaatggaggtacaggatatcacctgaattattaatgaatgcccaggaagtaattttcttctcattcttctaaaactactgcctttcaaagtgcacacacacgcgtccacatacactgcattcgttgctccagtataaattacatgcatgagcacctttctggcttttaagccaatataatgggctgcaaaatgaagacaccagagtgtatgcatacaaatctcactgtattaaagatgcaggttttctaattgtacccttcttgtctctctggcaatcttgcccttaatatccctggagttcctcatcagtgtcattttctgttatacacagttccacaattttgtctctagttgacttcaaatgtgtaactttattggtcttgccctattataattgtcatgactttcagattgtatctgaactcacagactgctgtcttactaataggtctggaaggtcacgctgaatgagaagtaaattattttatgtaaaaaaaaaaaaaaa

SEQ ID NO: 75 - DNA, 426 bp, Homo sapiens
  misc_feature (1)..(426): n is a, c, g, or t
cgttcattggtttattggcgaattacgcggtaagtggggctggggaactccaggacgggggaggggccgcagctgtcctgggggtcccggagcggcgctcacggtacttctcggtcaggacagaggatacgaccgatagaacttgtcccaggcggcgtgggctcggcgtngaagtcggcggggaagcgcgcggccagggtgaccagcaggcagtgggacaggagcttgaagttgaccgggtccacgcgcaggatgtaggcgtgcaactcgctcaagcttggacagggcgccgccgattgtcgtcgatgctcttcaccgcgtcgcccacgggcccaccacntttggaagccgtgcncgcgcaactgcgaagcncccgggttgcaggtcgaagttcgggaaagtaaggtcttggtctgcgggttggct

SEQ ID NO: 76 - DNA, 589 bp, Homo sapiens
accaaggccagtcctgagcaggcccaactccagtgcagctgcccaccctgccgccatgtctctgaccaagactgagaggaccatcattgtgtccatgtgggccaagatctccacgcaggccgacaccatcggcaccgagactctggagaggctcttcctcagccacccgcagaccaagacctacttcccgcacttcgacctgcacccggggtccgcgcagttgcgcgcgcacggctccaaggtggtggccgccgtgggcgacgcggtgaagagcatcgacgacatcggcggcgccctgtccaagctgagcgagctgcacgcctacatcctgcgcgtggacccggtcaacttcaagctcctgtcccactgcctgctggtcaccctggccgcgcgcttccccgccgacttcacggccgaggcccacgccgcctgggacaagttcctatcggtcgtatcctctgtcctgaccgagaagtaccgctgagcgccgcctccgggacccccaggacaggctgcggcccctcccccgtcctggaggttccccagccccacttaccgcgtaatgcgccaataaaccaatgaacgaagc

SEQ ID NO: 77 - DNA, 502 bp, Homo sapiens
  misc_feature (1)..(502): n is a, c, g, or t
tttttttttttcaccagcttatcaaatttattattccaatggcactagtacagctggaggtgctcatggtgacaccgcacaggacttcctgcctgctagaaatcatctacccgcgtgttcctttcccctttctggggcaaaagccactgcgggccatgtacccaaataaacctcttaatgcgtttgttaaaattagtttgggacatctgagtttccctctgaaggaaatgggaaaaagtgttatgggttgagtgatgggcacggcagcggggccccaggaaacctcttcaggggggaggcgtcgctggtgttcggggacagtctccagggtgttcgtctgaggantttcgagggacgtggggtgcttttggggtgctgcgagttccccggggncggcaggcgcttncagggaaggggccgcttacattttntccgggggagtttttncaggggcaggggatgctttttttttcagggccttncgttttgggggttttccgca

SEQ ID NO: 78 - DNA, 3148 bp, Homo sapiens
cgccgccatcctcggcgcgactcgcttctttcggttctacctgggagaatccaccgccatccgccaccatggtgaacttcacggtagaccagatccgcgccatcatggacaagaaggccaacatccgcaacatgtctgtcatcgcccacgtggaccatggcaagtccacgctgacagactccctggtgtgcaaggcgggcatcatcgcctcggcccgggccggggagacacgcttcactgatacccggaaggacgagcaggagcgttgcatcaccatcaagtcaactgccatctccctcttctacgagctctcggagaatgacttgaacttcatcaagcagagcaaggacggtgccggcttcctcatcaacctcattgactcccccgggcatgtcgacttctcctcggaggtgactgctgccctccgagtcaccgatggcgcattggtggtggtggactgcgtgtcaggcgtgtgcgtgcagacggagacagtgctgcggcaggccattgccgagcgcatcaagcctgtgctgatgatgaacaagatggaccgcgccctgctggagctgcagctggagcccgaggagctctaccagactttccagcgcatcgtggagaacgtgaacgtcatcatctccacctacggcgagggcgagagcggccccatgggcaacatcatgatcgatcctgtcctcggtaccgtgggctttgggtctggcctccacgggtgggccttcaccctgaagcagtttgccgagatgtatgtggccaagttcgccgccaagggggagggccagttggggcctgccgagcgggccaagaaagtagaggacatgatgaagaagctgtggggtgacaggtactttgacccagccaacggcaagttcagcaagtcagccaccagccccgaagggaagaagctgccacgcaccttctgccagctgatcctggaccccatcttcaaggtgtttgatgcgatcatgaatttcaagaaagaggagacagcaaaactgatagagaaactggacatcaaactggacagcgaggacaaggacaaagaaggcaaacccctgctgaaggctgtgatgcgccgctggctgcctgccggagacgccttgttgcagatgatcaccatccacctgccctcccctgtgacggcccagaagtaccgctgcgagctcctgtacgaggggcccccggacgacgaggctgccatgggcattaaaagctgtgaccccaaaggccctcttatgatgtatatttccaaaatggtgccaacctccgacaaaggtcggttctacgcctttggacgagtcttctcggggctggtctccactggcctgaaggtcaggatcatggggcccaactatacccctgggaagaaggaggacctctacctgaagccaatccagagaacaatcttgatgatgggccgctacgtggagcccatcgaggatgtgccttgtgggaacattgtgggcctcgtgggcgtggaccagttcctggtgaagacgggcaccatcaccaccttcgagcacgcgcacaacatgcgggtgatgaagttcagcgtcagccctgttgtcagagtggccgtggaggccaagaacccggctgacctgcccaagctggtggaggggctgaagcggctggccaagtccgaccccatggtgcagtgcatcatcgaggagtcgggagagcacatcatcgcgggcgccggcgagctgcacctggagatctgcctgaaggacctggaggaggaccacgcctgcatccccatcaagaaatctgacccggtcgtctcgtaccgcgagacggtcagtgaagagtcgaacgtgctctgcctctccaagtcccccaacaagcacaaccggctgtacatgaaggcgcggcccttccccgacggcctggccgaggacatcgataaaggcgaggtgtccgcccgtcaggagctcaagcagcgggcgcgctacctggccgagaagtacgagtgggacgtggctgaggcccgcaagatctggtgctttgggcccgacggcaccggccccaacatcctcaccgacatcaccaagggtgtgcagtacctcaacgagatcaaggacagtgtggtggccggcttccagtgggccaccaaggagggcgcactgtgtgaggagaacatgcggggtgtgcgcttcgacgtccacgacgtcaccctgcacgccgacgccatccaccgcggagggggccagatcatccccacagcacggcgctgcctctacgccagtgtgctgaccgcccagccacgcctcatggagcccatctaccttgtggagatccagtgtccagagcaggtggtcggtggcatctacggggttttgaacaggaagcggggccacgtgttcgaggagtcccaggtggccggcacccccatgtttgtggtcaaggcctatctgcccgtcaacgagtcctttggcttcaccgctgacctgaggtccaacacgggcggccaggcgttcccccagtgtgtgtttgaccactggcagatcctgcccggagaccccttcgacaacagcagccgccccagccaggtggtggcggagacccgcaagcgcaagggcctgaaagaaggcatccctgccctggacaacttcctggacaaattgtaggcggcccttcctgcagcgcctgccgccccggggactcgcagcacccacagcaccacgtcctcgaattctcagacgacacctggagactgtcccgacacagcgacgctcccctgagaggtttctggggcccgctgcgtgccatcactcaaccataacacttgatgccgtttctttcaatatttatttccagagtccggaggcagcagacacgccctcttagtagggacttaatgggccggtcggggagggggaggcgggatgggacacccaacactttttccatttcttcagagggaaactcagatgtccaaactaattttaacaaacgcattaagaggtttatttgggtacatggcccgcagtggcttttgccccagaaaggggaaaggaacacgcgggtagatgatttctagcaggcaggaagtcctgtgcggtgtcaccatgagcacctccagctgtactagtgccattggaataataaatttgataaggtggtgaaaaaa

SEQ ID NO: 79 - DNA, 417 bp, Homo sapiens
  misc_feature (1)..(417): n is a, c, g, or t
tttttttttccntttaaagtcatctctataggaaggtnctgggagggatcccagagaaagaaagggccaagactccattaactgccctggatgaagggactgctacaanagctagtaccagagactctcctatctcacggttgaggcagacccaggntagaatagagaataaaaggaatgcttataggaaacaattttgtatggaatgctagatggccaagctcagcctttngtccagtgcaacccttgcctcgcttgtcaacagtgaaaaattagtttngttagaagaaccatctggaaacacaccagcttctgctaccttcatgctcattgttaaaaaaagattaaccagtgtgaacattctgatctgttaattcaggggactnttttctttccaatggacttttttgttgggag

SEQ ID NO: 80 - DNA, 2614 bp, Homo sapiens
attgcggagagcggctgatcgcagtccggaggtgaggcggaactctgagcaggtggtccattatggctgacatgcaaaatctggtagaaagattggagagggcagtgggccgcctggaggcagtatctcatacctctgacatgcaccgtgggtatgcagacagtccttcaaaagcaggagcagctccatatgtgcaggcatttgactcgctgcttgctggtcctgtggcagagtacttgaagatcagtaaagagattgggggagacgtgcagaaacatgcggagatggtccacacaggtttgaagttggagcgagctctgttggttacagcttctcagtgtcaacagccagcagaaaataagctttccgatttgttggcacccatctcagagcagatcaaagaagtgataacctttcgggagaagaaccgaggcagcaagttgtttaatcacctgtcagctgtcagcgaaagtatccaggccctgggctgggtggctatggctcccaagcctggcccttatgtgaaagaaatgaatgatgccgccatgttttatacaaaccgagtcctcaaagagtacaaagatgtggataagaagcatgtagactgggtcaaagcttatttaagtatatggacagagctgcaggcttacattaaggagttccataccaccggactggcctggagcaaaacggggcctgtggcaaaagaactgagcggactgccatctggaccctctgccggatcaggtcctcctccccctccaccaggcccccctcctcccccagtctctaccagttcaggctcagatgagtctgcttcccgctcagcactgttcgcgcagattaatcagggggagagcattacacatgccctgaaacatgtatctgatgacatgaagactcacaagaaccctgccctgaaggctcagagtggtccagtacgcagtggccccaaaccattctctgcacctaaaccccaaaccagcccatcccccaaacgagccacaaagaaggagccagctgtacttgaactggagggcaagaagtggagagtggaaaatcaggaaaatgtttccaacctggtgattgaggacacagagctgaaacaggtggcttacatatacaagtgtgtcaacacgacattgcaaatcaagggcaaaattaactccattacagtagataactgtaagaaacttggcctggtattcgatgacgtggtgggcattgtggagataatcaacagtaaggatgtcaaagttcaggtaatgggtaaagtgccaaccatatccatcaacaaaacagatggctgccatgcttacctgagcaagaattccctggattgtgaaatagtcagtgccaaatcttccgagatgaatgtcctcattcctacagaaggcggtgactttaatgaattcccagttcctgagcagttcaagaccctatggaacgggcagaagttggtcaccacagtgacagaaattgctggataagcgaagtgccactgggttctttgccctcccttcacaccatgggataaatctgtatcaagacggttcttttctagatttcctctacctttttgctcttaaaactgcttctctgctctgagaagcacagctacctgccttcactgaaatatacctcaggctgagatttggggtgggatagcaggtcagttgatcttctgcaggaaggtgcagcttttccatatcagctcaaccacgccgccagtccattcttaaggaactgccgactaggactgatgatgcattttagctttgagcttttgggggttattctaccaacaaacagtccattggaaagaaaacagtccctggaattaacagatcagaatgttcacactggttaatctttttttaacaatgagcatgaaggtagcagaagctggtgtgtttccagatggttcttctaaccaaactaatttttcactgttgacaagcgaggcaagggttgcactggaccaaaggctgaggcttggccatctagcattccatacaaaattgtttcctataagcattccttttattctctattctatcctgggtctgcctcaaccgtgagataggagagtctctggtactagctgctgtagcagtgcccttcatccagggcagttaatggagtcttggaccctttctttctctgggatccctgcccagcaccttcctatagagatgactttaaaaggaaaaaaaaaaaaaaacccacatgatttcaaggagtctggcattcctgaatccttcttccctgccaggtgcctgtcacctgtcttcactgcctccttttccctgtcatgctcatcagcttatggcttctgtctaagcacctgaacagaggactgaaacctccactgcaggctggttttaggtcttgaattatgtaagaatcttgcacagcactgctaatgtaaatttcagttgtttttccctctaggacaaacacttaccaaaatatgcaacttttttttggtgggaagagagattgtcctgtgatttctacccatttcctgaggcctgtggaaataaacctttatgtacttaaagttatacagaaaatagaataaagttaataccaaacttgaaaaagcggccgc

SEQ ID NO: 81 - DNA, 543 bp, Homo sapiens
ggaactaaaaaagaactttatttattgagggcaaggggatgcaaacaatacaaaaatcaaaagcttatctggtatttaacttttctttctctgcttgtcaaatgagagttagattttatttttacatttgctaagtgtcctgatctgctcatgaaatccttctatgggggaagctgtggggcagattccttaagcgaccctttgggacaactcttatcagggaggagcgaactgctcatttctgcctacttctttcccttctgcttcatgtgtactacaaaatagtcattgcatgcaatggtgaggcccgcaattagggaaaagaagctctggaagcccactttgccatctctacactggtccaggtccttcattattttgtccacagccagagggtctttttgattttccaaaaatccagggaactccttttccatgagtactctcaggtcctcctttgttaagtaggctttatccccagcgaatttgttgaatgttaacatcatggtttccatggcaggttccatttgagaagtcattttg

SEQ ID NO: 82 - DNA, 649 bp, Homo sapiens
agaatacactcacaagccactccgctgctcgcctctccgccccgcgtccagctcgcccagctcgcccagcgtccgccgcgcctcgccaaggcttcaacggaccacaccaaaatgccatctcaaatggaacacgccatggaaaccatgatgtttacatttcacaaattcgctggggataaaggctacttaacaaaggaggacctgagagtactcatggaaaaggagttccctggatttttggaaaatcaaaaagaccctctggctgtggacaaaataatgaaggacctggaccagtgtagagatggcaaagtgggcttccagagcttcttttccctaattgcgggcctcaccattgcatgcaatgactattttgtagtacacatgaagcagaagggaaagaagtaggcagaaatgagcagttcgctcctccctgataagagttgtccaaagggtcgcttaaggaatctgccccacagcttcccccatagaaggatttcatgagcagatcaggacacttagcaaatgtaaaaataaaatctaactctcatttgacaagcagagaaagaaaagttaaataccagataagcttttgatttttgtattgtttgcatccccttgccctcaataaataaagttcttttttagttcc

SEQ ID NO: 83 - DNA, 565 bp, Homo sapiens
  misc_feature (1)..(565): n is a, c, g, or t
acagatatgaaaaagcaagaaggcaaagtgagagaaacagatgctacccacagggcagcggtggcatttggttgttttggtctttgtaccagcaattcccactcccgaggcagataatccacttcctgccagactacacagtttttcttgcagtcaagacacgaacaatggaccctactcctccagcagcaagtgccttttcatgccattgcagtaacatagcactgctattgtcagacgggctctcaaatcctttataaatatacttcattaggagatccacaccattcttgtccagagattgaactgccttttctatatcattagctttaaaagagatgagcaccttcaagacaatgctgcctgcccggtccttcactgcctgactcttggtgttgataggggggttcttcagagctgcctgtagggactggttcatgtttccttggcgcanggggaaggttccacctcggcctcgtcggggcccgggcctgggcgtncggcccccatcttcttcggtccacgaacttgttctcgtcaaaattcatccaangtccaccttccg

SEQ ID NO: 84 - DNA, 2000 bp, Homo sapiens
gggagcgcgaacctgaggaggaagaaacggggctagcgcgcaggcccagaacggtccgagccgcggcagtcggcgacgcctcagagcggaagagggaagtgaatcaggcgccgggtagtgggttgctgggctgggcttgctgaggtagaggcagcgccaagaagaggcctttgccgctggtcgggattgggatgtcgaagaacacagtgtcgtcggcccgcttccggaaggtggacgtggatgaatatgacgagaacaagttcgtggacgaagaagatgggggcgacggccaggccgggcccgacgagggcgaggtggactcctgcctgcggcaaggaaacatgacagctgccctacaggcagctctgaagaacccccctatcaacaccaagagtcaggcagtgaaggaccgggcaggcagcattgtcttgaaggtgctcatctcttttaaagctaatgatatagaaaaggcagttcaatctctggacaagaatggtgtggatctcctaatgaagtatatttataaaggatttgagagcccgtctgacaatagcagtgctatgttactgcaatggcatgaaaaggcacttgctgctggaggagtagggtccattgttcgtgtcttgactgcaagaaaaactgtgtagtctggcaggaagtggattatctgcctcgggagtgggaattgctggtacaaagaccaaaacaaccaaatgccaccgctgccctgtgggtagcatctgtttctctcagctttgccttcttgctttttcatatctgtaaagaaaaaaattacatatcagttgtcctttaatgaaaattgggataatatagaagaaattgtgttaaaatagaagtgtttcatcctttcaaaaccatttcagtgatgtttataccaatctgtatatagtataatttacattcaagtttaattgtgcaacttttaacccctgttggctggttttttgttctgttttgttttgtattatttttaactaatactgagagatttggtcagaatttgaggccagtttcctagctcattgctagtcaggaaatgatatttataaaaaatatgagagactggcagctattaacattgcaaaactggaccatatttcccttatttaataagcaaaatatgtttttggaataagtggtgggtgaataccactgctaagttatagctttgtttttgcttgcctcctgattatctgtactgtgggtttaagtatgctactttctctcagcatccaataatcatggcccctcaatttatttgtggtcacccagggttcagagcaagaagtcttgctttatacaaatgtatccataaaatatcagagcttgttgggcatgaacatcaaacttttgttccactaatatggctctgtttggaaaaaactgcaaatcagaaagaatgatttgcagaaagaaagaaaaactatggtgtaatttaaactctgggcagcctctgaatgaaatgctactttctttagaaatataatagctgccttagacattatgaggtatacaactagtatttaagataccatttaatatgccccgtaaatgtcttcagtgttcttcagggtagttgggatctcaaaagatttggttcagatccaaacaaatacacattctgtgttttagctcagtgttttctaaaaaaagaaactgccacacagcaaaaaattgtttactttgttggacaaaccaaatcagttctcaaaaaatgaccggtgcttataaaaagttataaatatcgagtagctctaaaacaaaccacctgaccaagagggaagtgagcttgtgcttagtatttacattggatgccagttttgtaatcactgacttatgtgcaaactggtgcagaaattctataaactctttgctgtttttgatacctgctttttgtttcattttgttttgttttgtaaaaatgataaaacttcagaaaataaaatgtcagtgttgaataaaaaaaaaaaaaaaaaaaa

SEQ ID NO: 85 - DNA, 323 bp, Homo sapiens
ttcacagttattgttttattcttgttctcttgtgtcgtttacagtgtctctcacaaaagaagaactccctctggcaggggatgtgactagggactcattgggccagcaacaaccacaaaagaatctaggcattagaatgaactactggaacctgagtcaaagacctgttagtgataatttgcccattttctggcctctagttgcttgaatacgggtaagacagggaaatattaaatgattgtgtactgagatggagacgtggaagatctggccctgatggaggatcagagggagcaggttgggtgaaagcccggttttaagcc

SEQ ID NO: 86 - DNA, 1249 bp, Homo sapiens
ggcacgagccagggtttcctcttcaagtaggtctaaaacattttttttctcattgacttccttcctgttctaactgccagtactcagaagtcagagttgagagacagaggcaccccggacagagacgtgaagcactgaataaatagatcagaatgactgaaaaagccccagagccacatgtggaggaggatgacgatgatgagctggacagcaagctcaattataagcctccaccacagaagtccctgaaagagctgcaggaaatggacaaagatgatgagagtctaattaagtacaagaaaacgctgctgggagatggtcctgtggtgacagatccgaaagcccccaatgtcgttgtcacccggctcaccctggtttgtgagagtgccccgggaccaatcaccatggaccttactggagatctggaagccctcaaaaaggaaaccattgtgttaaaggaaggttctgaatatagagtcaaaattcacttcaaagtgaacagggatattgtgtcaggcctgaaatacgttcagcacacctacaggactggggtgaaagtggataaagcaacatttatggttggcagctatggacctcggcctgaggagtatgagttcctcactccagttgaggaggctcccaagggcatgctggcccgaggcacgtaccacaacaagtccttcttcaccgacgatgacaagcaagaccacctcagctgggagtggaacctgtcgattaagaaggagtggacagaatgaatgcatccaccccgttccccacccttgccacctggaagaattctctcaggcgtgttcagcaccctgtccctcctccctgtccacagctgggtccctcttcaacactgccacatttccttattgatcgatcttttcccaccctgtcactcaacgtggtccctagaacaagaggcttaaaaccgggctttcacccaacctgctccctctgatcctccatcagggccagatcttccacgtctccatctcagtacacaatcatttaatatttccctgtcttacccctattcaagcaactagaggccagaaaatgggcaaattatcactaacaggtctttgactcaggttccagtagttcattctaatgcctagattcttttgtggttgttgctggcccaatgagtccctagtcacatcccctgccagagggagttcttcttttgtgagagacactgtaaacgacacaagagaacaagaataaaacaataactgtg

SEQ ID NO: 87 - DNA, 325 bp, Homo sapiens
gagcctagcaagcagtttagatggactacattcctcattccagctgaggagagatctcacatggagaaaggagtgctaaggaagagcaagaccccacagccttccaaagatcccctatggtccaaagtcctttgaccatctcagtcagctgctttgttttctgttggcagttgagggacaaggtgagaggagccaggggtagtcatgaacaccagtgggttctgccctgggcagctccccaccttctttaagagagtactgtgtctcagctccagcagtctcaactgggaagacccaggactcctgctcttttctctaatccctg

SEQ ID NO: 88 - DNA, 1468 bp, Homo sapiens
ggcctgcgcctccacgtggatttggtagagaaaccccggactgggatcatggcagccgagactcggaacgtggccggagcagaggccccaccgccccagaagcgctactaccggcaacgtgctcactccaaccccatggcggaccacacgctgcgctaccctgtgaagccagaggagatggactggtctgagctatacccagagttcttcgctccactcactcaaaatcagagccacgatgacccaaaggataagaaagaaaagagagctcaggcccaagtggagtttgcagacataggctgtggctatggtggcctgttagtggaactgtcaccgctgttcccagacacacttattctgggtctggagatccgggtgaaggtctcagactatgtacaagaccggattcgggccctacgcgcagctcctgcaggtggcttccagaacatcgcctgtctccgtagcaatgccatgaagcaccttcctaacttcttctacaagggccagctgacaaagatgttcttcctcttccccgacccacatttcaagcggacaaagcacaagtggcgaatcatcagtcccaccctgctagcagaatatgcctacgtgctaagagttggggggctggtgtataccataaccgatgtgctggagctacacgactggatgtgcactcatttcgaagagcacccactgtttgagcgtgtgcctctggaggacctgagtgaagaccccgttgtgggacatctaggcacctcaactgaggaggggaagaaagttctacgtaatggagggaagaatttcccagccatcttccgaagaatacaagatcccgtcctccaggcagtgacctcccaaaccagcctgcctggtcactgactgcttactctaccttagctggacctcgtctcccagggattagagaaaagagcaggagtcctgggtcttcccagttgagactgctggagctgagacacagtactctcttaaagaaggtggggagctgcccagggcagaacccactggtgttcatgactacccctggctcctctcaccttgtccctccactgccaacagaaaacaaagcagctgactgagatggtcaaaggactttggaccataggggatctttggaaggctgtggggtcttgctcttccttagcactcctttctccttgtgagatctctcctcagctggaatgaggaatgtagtccatctaaactgcttgctaggctcaattaccacttctgtttgctttgtggatcctgggataacatgtatatgtgtacacatgcctattctgccatttctcaggaaagggtggagtatgtatcatatgccttctaccatcctcctgtcctctccctgcgtaagacttgacctggggaatctgtgttccctaaaaaaaaaaaaaaaaaaaaaaaaaaaaaaaaaaaaaaaaaaaaaaaaaaaaaaaaaaaaaaaaaa

SEQ ID NO: 89 - DNA, 392 bp, Homo sapiens
  misc_feature (1)..(320): n is a, c, g, or t
attcggcacaggnccgnctntaccactcttctctcatggatcctgataccaaactcatcggaaacatggcactgttgcctatcagaagtcaattcaaaggacctgcccccagagagacaaaagatacagatattgtggatgaagccatctattacttcaaggccaatgtcttcttcaaaaactatgaaattaagaatgaagctgataggaccttgatatatataactctctacatttctgaatgtctgaagaaactgcaaaagtgcaattccaaaagccaaggtgagaaagaaatgtatacgctgggaatcactaattttcccattcctgggagagcctgggttttccactttaacggcaatttatgcccaaacctgcaaacaaacagggaa

SEQ ID NO: 90 - DNA, 912 bp, Homo sapiens
cgcttccgcctacctcgcccaggctgccagaccggaagcgctccgctgtacctggatcctgctcctctgggttgaaacccgggcgccgccaagatgccggcttaccactcttctctcatggatcctgataccaaactcatcggaaacatggcactgttgcctatcagaagtcaattcaaaggacctgcccccagagagacaaaagatacagatattgtggatgaagccatctattacttcaaggccaatgtcttcttcaaaaactatgaaattaagaatgaagctgataggaccttgatatatataactctctacatttctgaatgtctgaagaaactgcaaaagtgcaattccaaaagccaaggtgagaaagaaatgtatacgctgggaatcactaattttcccattcctggagagcctggttttccacttaacgcaatttatgccaaacctgcaaacaaacaggaagatgaagtgatgagagcctatttacaacagctaaggcaagagactggactgagactttgtgagaaagttttcgaccctcagaatgataaacccagcaagtggtggacttgctttgtgaagagacagttcatgaacaagagtctttcaggacctggacagtgaagggagcccgggcagccaccgtctccagagccctgggcagcattttccagcaagatgtacacaatcttttgcctttatttcgtaaagttttatacagaagagagaagagcatgtctttacttgaaaaactcttgatcaagaatttgggtgggagaaaagaaagtgggttatcaagggtgatttgaaattttctgcagcattaaagctggcgcttaataagaataagtaataataaagaaatttctaacattccaaaaaaaaaaaaaaaaaaaaaaaaaaaaa

SEQ ID NO: 91 - DNA, 369 bp, Homo sapiens
ttttttttttttttcctggtccacagaagatgtttatttgatgtaacaaaacaatacagttagtgttagatccaaccacaaagagcaaaaggaatgaaaaatttgtacaatgtacatagaaaaaacaagatatttactgtgacaactatatattcctaaaataatgcttctaaaattactcaattattgaaatctacatgaaaaaaaggatgttaatagcgacaaagtgcataaaatcaaacattgtattttgagcaaattaacatactaggcaattttgctaagaatgcatgattttttttttcttgtttacagtctgctcaaaatatctttataccaacagggtaggcagaacatttaggtttaata

SEQ ID NO: 92 - DNA, 3933 bp, Homo sapiens
cacgaggcagcactctcttcgtcgcttcggccagtgtgtcgggctgggccctgacaagccacctgaggagaggctcggagccgggcccggaccccggcgattgccgcccgcttctctctagtctcacgaggggtttcccgcctcgcacccccacctctggacttgcctttccttctcttctccgcgtgtggagggagccagcgcttaggccggagcgagcctgggggccgcccgccgtgaagacatcgcggggaccgattcaccatggagggcgccggcggcgcgaacgacaagaaaaagataagttctgaacgtcgaaaagaaaagtctcgagatgcagccagatctcggcgaagtaaagaatctgaagttttttatgagcttgctcatcagttgccacttccacataatgtgagttcgcatcttgataaggcctctgtgatgaggcttaccatcagctatttgcgtgtgaggaaacttctggatgctggtgatttggatattgaagatgacatgaaagcacagatgaattgcttttatttgaaagccttggatggttttgttatggttctcacagatgatggtgacatgatttacatttctgataatgtgaacaaatacatgggattaactcagtttgaactaactggacacagtgtgtttgattttactcatccatgtgaccatgaggaaatgagagaaatgcttacacacagaaatggccttgtgaaaaagggtaaagaacaaaacacacagcgaagcttttttctcagaatgaagtgtaccctaactagccgaggaagaactatgaacataaagtctgcaacatggaaggtattgcactgcacaggccacattcacgtatatgataccaacagtaaccaacctcagtgtgggtataagaaaccacctatgacctgcttggtgctgatttgtgaacccattcctcacccatcaaatattgaaattcctttagatagcaagactttcctcagtcgacacagcctggatatgaaattttcttattgtgatgaaagaattaccgaattgatgggatatgagccagaagaacttttaggccgctcaatttatgaatattatcatgctttggactctgatcatctgaccaaaactcatcatgatatgtttactaaaggacaagtcaccacaggacagtacaggatgcttgccaaaagaggtggatatgtctgggttgaaactcaagcaactgtcatatataacaccaagaattctcaaccacagtgcattgtatgtgtgaattacgttgtgagtggtattattcagcacgacttgattttctcccttcaacaaacagaatgtgtccttaaaccggttgaatcttcagatatgaaaatgactcagctattcaccaaagttgaatcagaagatacaagtagcctctttgacaaacttaagaaggaacctgatgctttaactttgctggccccagccgctggagacacaatcatatctttagattttggcagcaacgacacagaaactgatgaccagcaacttgaggaagtaccattatataatgatgtaatgctcccctcacccaacgaaaaattacagaatataaatttggcaatgtctccattacccaccgctgaaacgccaaagccacttcgaagtagtgctgaccctgcactcaatcaagaagttgcattaaaattagaaccaaatccagagtcactggaactttcttttaccatgccccagattcaggatcagacacctagtccttccgatggaagcactagacaaagttcacctgagcctaatagtcccagtgaatattgtttttatgtggatagtgatatggtcaatgaattcaagttggaattggtagaaaaactttttgctgaagacacagaagcaaagaacccattttctactcaggacacagatttagacttggagatgttagctccctatatcccaatggatgatgacttccagttacgttccttcgatcagttgtcaccattagaaagcagttccgcaagccctgaaagcgcaagtcctcaaagcacagttacagtattccagcagactcaaatacaagaacctactgctaatgccaccactaccactgccaccactgatgaattaaaaacagtgacaaaagaccgtatggaagacattaaaatattgattgcatctccatctcctacccacatacataaagaaactactagtgccacatcatcaccatatagagatactcaaagtcggacagcctcaccaaacagagcaggaaaaggagtcatagaacagacagaaaaatctcatccaagaagccctaacgtgttatctgtcgctttgagtcaaagaactacagttcctgaggaagaactaaatccaaagatactagctttgcagaatgctcagagaaagcgaaaaatggaacatgatggttcactttttcaagcagtaggaattggaacattattacagcagccagacgatcatgcagctactacatcactttcttggaaacgtgtaaaaggatgcaaatctagtgaacagaatggaatggagcaaaagacaattattttaataccctctgatttagcatgtagactgctggggcaatcaatggatgaaagtggattaccacagctgaccagttatgattgtgaagttaatgctcctatacaaggcagcagaaacctactgcagggtgaagaattactcagagctttggatcaagttaactgagctttttcttaatttcattcctttttttggacactggtggctcactacctaaagcagtctatttatattttctacatctaattttagaagcctggctacaatactgcacaaacttggttagttcaatttttgatcccctttctacttaatttacattaatgctcttttttagtatgttctttaatgctggatcacagacagctcattttctcagttttttggtatttaaaccattgcattgcagtagcatcattttaaaaaatgcacctttttatttatttatttttggctagggagtttatccctttttcgaattatttttaagaagatgccaatataatttttgtaagaaggcagtaacctttcatcatgatcataggcagttgaaaaatttttacaccttttttttcacattttacataaataataatgctttgccagcagtacgtggtagccacaattgcacaatatattttcttaaaaaataccagcagttactcatggaatatattctgcgtttataaaactagtttttaagaagaaattttttttggcctatgaaattgttaaacctggaacatgacattgttaatcatataataatgattcttaaatgctgtatggtttattatttaaatgggtaaagccatttacataatatagaaagatatgcatatatctagaaggtatgtggcatttatttggataaaattctcaattcagagaaatcatctgatgtttctatagtcactttgccagctcaaaagaaaacaataccctatgtagttgtggaagtttatgctaatattgtgtaactgatattaaacctaaatgttctgcctaccctgttggtataaagatattttgagcagactgtaaacaagaaaaaaaaaatcatgcattcttagcaaaattgcctagtatgttaatttgctcaaaatacaatgtttgattttatgcactttgtcgctattaacatcctttttttcatgtagatttcaataattgagtaattttagaagcattattttaggaatatatagttgtcacagtaaatatcttgttttttctatgtacattgtacaaatttttcattccttttgctctttgtggttggatctaacactaactgtattgttttgttacatcaaataaacatcttctgtggaaaaaaaaaaaaaaaaaaaa

SEQ ID NO: 93 - DNA, 282 bp, Homo sapiens
  misc_feature (1)..(282): n is a, c, g, or t
gctccccagtggtcagcggagaccccaaggaggatcacaactacagcagtgccaagtcctccaacgcccggagcacctcgcccaccagcgactccatctcctcctcctcctcctcagccgacgaccactatgagtttgccaccaaggggagccaggagggcagcgagggcagcgaggggagcttccggagccacgagagccccagcgacacggaagaggacgacaggaagnacagccagaaggagcccaaggattttttnggggacagcgggtacgattncc

SEQ ID NO: 94 - DNA, 2667 bp, Homo sapiens
gagggtgggtcgaccccgggaattcggcacgagcggcggcggggccacccgcgagtccagcgtcgccgcagccccccaatgcggccgcgagaagcagcgggggggcaggcgatcgaaggagccttcacgtaaatgggtccagtcatgcctcccagtaagaagccagaaagctcaggaattagtgtctccagtggactgagtcagtgttacgggggcagcggtttctccaaggcccttcaggaagacgatgacctcgacttttctctgcctgacatccgattagaagagggggccatggaagatgaagagctgaccaacctgaactggctgcacgagagcaagaacttgctgaagagctttggggagtcggtcctcaggagtgtcagccccgtccaggacctggacgatgacacccccccatcccctgcccactctgacatgccctacgatgccaggcagaaccccaactgcaaacccccctactccttcagctgcctcatatttatggccatcgaggactctccaaccaagcgcctgccagtgaaggatatctacaactggatcttggaacattttccgtattttgcaaatgcacctactgggtggaaaaactcagtgagacacaatttatcattgaataagtgttttaagaaagtggacaaagagaggagtcagagtattgggaaagggtcgttgtggtgcatagacccagagtatagacaaaatctaattcaggctttgaaaaagacaccttatcacccacacccacacgtgttcaatacacctcccacctgtcctcaggcatatcaaagcacatcaggtccacccatctggccgggcagtaccttcttcaagagaaatggagcccttctccaagatcctgacattgatgctgccagtgccatgatgcttttgaatactccccctgagatacaagcaggttttcctccaggagtgatccaaaatggagcgcgggtcctgagccgagggctgtttcctggcgtgcggccgctgccaatcactcccattggggtgacagcggccatgaggaatggcatcaccagctgccggatgcggactgagagtgagccatcttgtggctccccagtggtcagcggagaccccaaggaggatcacaactacagcagtgccaagtcctccaacgcccggagcacctcgcccaccagcgactccatctcctcctcctcctcctcagccgacgaccactatgagtttgccaccaaggggagccaggagggcagcgagggcagcgaggggagcttccggagccacgagagccccagcgacacggaagaggacgacaggaagcacagccagaaggagcccaaggattctctgggggacagcgggtacgcatcccagcacaagaagcgccagcacttcgccaaggccaggaaggtccccagcgacacactgcccctcaaaaagagacgcaccgaaaagccccccgagagcgatgatgaggagatgaaagaagcggcagggtccctcctgcacttagcagggatccggtcctgtttgaataacatcaccaatcggacggcaaaggggcagaaagagcaaaaggaaaccacaaaaaattaaaaacaagtcactgatttgttttgaacttacgaccatttggtttcagcatgtcaggagatttctaatgatttgtggcaatatcagcaattttttttcttttttcttgtttggggtttggttttctttcttttcttttccttttattgggttttaatttgccccctcttctttgttttggacccttaagaattttatttttaaaggagattgaagccatagaactcatattgacactcagctgttttacaaaagcttttcattatctgaagacaaaaccgaaaaagccaaaattaccattgcttcctccagcttgtcagaaacctgtggctgaatccgcagggatgtcaacgtcaatatcacaggaacacacattcggcacctagaaggcacgtgggcaaagtaatcatcgttcaggcccaacccttaggtttaaaaagtcaggttgtccatcccattggggttcactgagtgaaggcacataaagcaattgaggaggaggaggaacccctcgtccccctaggagcagacccaagcttgtggcaccaggcatctgatggtgccaggaaagccactggaattgtcacacggcgagcacagagggccggccaccagtcctcgatgcttctgaaccctgaaccccgatgacatcttacgaggtggacgttggactgttcatgcgcatcgggtgtcagtgactcatggagaagaaatggggtaaatttttagtgatgttgctaatcattgaattctgttctctattaaattaagaaaatgttccaaaagccataagcctgaagattggccctgtgcacgcacgcacacacacacacacacacacacacacacacacacacacacgaaggagagagagagaaaactgatggggaaaacaagctgtgtcttcttaactgcccaagtgaaaagcaaccaagtccaggaaattacaatagctgttaaggaaaggaaataatggtacagatctttttctgtctatcaaaactatttgatccaagtgaaaaaaaaaaaaaaactagaaagctacggaacctgcaatgcggccgc

(19) United States
(12) Patent Application Publication
(10) Pub. No.: US 2007/0231791 A1
(43) Pub. Date: Oct. 4, 2007

(54) Gene Equation to Diagnose Rheumatoid Arthritis

(76) Inventors: Nancy J. Olsen, Dallas, TX (US); Thomas M. Aune, Franklin, TN (US)

Correspondence Address: JENKINS, WILSON, TAYLOR & HUNT, P. A., SUITE 1200, UNIVERSITY TOWER, 3100 TOWER BOULEVARD, DURHAM, NC, 27707, US

(21) Appl. No.: 10/555,734

(22) PCT Filed: May 10, 2004
(86) PCT No.: PCT/US04/14618

§ 371 Date: Feb. 13, 2007

Related U.S. Application Data
(60) Provisional application No. 60/468,901, filed on May 8, 2003.

(51) Int. Cl. C12Q 1/68

(52) U.S. Cl. 435/6

(57) ABSTRACT

The presently clamied subject matter provides a method for detecting a predisposition to developing established rheumatoid arthritis (RA) in a subject by obtaining a biological sample from the subject, determining expression levels of at least two genes in the biological sample, and comparing the expression level of each gene with a standard, wherein the comparing detects a predisposition to developing established RA in the subject. Also provided are compositions and kits for carrying out the methods of the presently claimed subject matter.

### CROSS REFERENCE TO RELATED APPLICATIONS

The present patent application is based on and claims priority to U.S. Provisional Application Ser. No. 60/468,901, entitled “A GENE EQUATION TO DIAGNOSE RHEUMATOID ARTHRITIS”, which was filed May 8, 2003 and is incorporated herein by reference in its entirety.

### GRANT STATEMENT

This work was supported by grants from the Arthritis Foundation, the Juvenile Diabetes Foundation, and by a Vanderbilt University Medical Center Discovery Grant. Additionally, this work was supported by grants A144924, AR02027, AR41943, and DK58765 from the U.S. National Institutes of Health. Thus, the U.S. government has certain rights in the claimed subject matter.

### TECHNICAL FIELD

The presently claimed subject matter generally relates to the diagnosis of rheumatoid arthritis (RA). More specifically, the claimed subject matter relates to identifying a predisposition to developing established RA.

### TABLE OF ABBREVIATIONS

6-JOE—6-carboxy-4′,5′-dichloro-2′,7′-dimethoxyfluorescein, succinimidyl ester
aaRNA—Amplified Antisense RNA
Acc. No.—GenBank Accession Number
Ag—antigen
ARHGDIB—rho GDP dissociation inhibitor
ARPC3—actin-related protein subunit 3
ARPC5—actin-related protein subunit 5
B2M—β2-microglobulin
BMP4-bone morphogenic protein 4
CAP—adenylyl cyclase-associated protein
CCR1—chemokine receptor (c-c motif)
cDNA—complementary DNA
CHES1—checkpoint suppressor 1
CHI2L1—chitinase 3-like 1 (cartilage glycoprotein-39)
CSF3R—colony stimulating factor 3 receptor (granulocyte)
CSTF2—cleavage stimulation factor subunit 2
CYP24A1—cytochrome P450 subfamily 24
CYP3A4—cytochrome P450 subfamily 3A4
DMARDs—disease modifying anti-rheumatic drugs
DOP-PCR—Degenerate Oligonucleotide Primed PCR
EEF2—translation elongation factor 2
EST—expressed sequence tag
FITC—fluorescein isothiocyanate
FKBP1A—FK506 binding protein 1A
FYB—FYN-binding protein
GMBS—gamma-maleimidobutyryloxy-succimide
HBZ—hemoglobin zeta
HIF1A—hypoxia inducible factor
HLA-DPA1—MHC class II DP α1
HLA-DRA—MHC class II DR α
HSD11B2—human 11-β hydroxysteroid dehydrogenase 2
IDDM—insulin-dependent (type I) diabetes mellitus
IFI30—interferon gamma-inducible protein 30
LabMAP—Laboratory Multiple Analyte Profiling
LAMR1—67 kD laminin receptor 1
LASP-1—LIM and SHE protein-1
LCP-1—L-plastin
LMAN1—mannose-binding lectin 1
LTBP1—latent TGF-β binding protein 1
M17S2—ovarian carcinoma antigen (CA-125)
METTL1—methyltransferase-like 1
MHC—major histocompatability complex
MS—multiple sclerosis
NK cell—natural killer cell
NKT cell—natural killer T cell
NSAIDs—nonsteroidal anti-inflammatory drugs
NSEP1—nuclease sensitive element binding protein
OAZ1—ornithine decarboxylase Antizyme 1
PCR—polymerase chain reaction
PEP—Primer-Extension Pre-amplification
PMBC—peripheral blood mononuclear cell(s)
POR—cytochrome P450 oxidoreductase
PTGES—human prostaglandin E synthase
PTPRA—protein tyrosine phosphatase
RA—rheumatoid arthritis
RAB7—RAS oncogene family
RAPD—rapid amplification of polymorphic DNA
RGS4-regulator of G-protein signaling 4
RP-PCR—random-primed PCR
RT-PCR—reverse transcription PCR
S100A10—calpactin 1
SAS—sarcoma amplified sequence
SAT—spermidine/spermine N1-acetyltransferase
SD—standard deviation(s)
SEM—standard error of the mean
SISPA—Sequence-Independent, Single-Primer Amplification
SLE—systemic lupus erythematosus
SNTA1—syntrophin α, neuromuscular junction
SNX2—sorting nexin 2
SSX3—synovial sarcoma breakpoint 3
TGFBR2—transforming growth factor β receptor II
TNF-α—tumor necrosis factor-α
TNNI2—troponin I, skeletal, fast twitch protein
TNNT2—cardiac troponin T2
ZFP36L1—EGF-response factor 1
ZNF74—zinc finger protein 74

### BACKGROUND ART

Rheumatoid arthritis (RA) is an autoimmune disease suffered by millions of patients in the United States alone, with a total annual cost of billions of dollars a year. RA is characterized by progressive inflammation of the synovial lining of the joints, leading to pain, stiffness, swelling, and debilitating joint destruction. As such, there is an ongoing need to discover techniques to rapidly and accurately diagnose patients with RA.

The importance of the need for a rapid and accurate diagnostic test for RA, as well as for other autoimmune diseases, is underscored by changes in the approaches to treatment of these diseases. Until recently, rheumatologists initiated therapy for a newly diagnosed patient with nonsteroidal anti-inflammatory drugs (NSAIDs) and low dose corticosteroids. As the disease progressed, additional disease modifying anti-rheumatic drugs (DMARDS) were added. Rheumatologists now recognize that early and aggressive therapy with newer agents such as methotrexate, leflunomide, or the new tumor necrosis factor-α (TNF-α) inhibitors (for example, etanercept and infliximab) can provide improved outcomes and can preserve function and improve quality of life. See Jacobson et al., 1997. However, these newer drugs are expensive and are characterized by side effects. Thus, such drugs should be used in patients that clearly have RA, especially forms of RA that are likely to develop into the chronic, established form of the disease.

Therefore, improved diagnostic tests that can readily detect those molecular changes associated with an increased risk for developing established RA are needed. The need for this type of diagnostic test is substantial, since approximately 1% of the population has RA (Kukreja & Maclaren, 2000) and physicians might suspect the disease in at least twice that number (Ufret-Vincenty et al., 1998). To address this need, the presently claimed subject matter provides in one embodiment a method for diagnosing in a subject a predisposition to developing established RA.

### SUMMARY

The presently claimed subject matter provides methods and compositions for detecting a predisposition to developing established rheumatoid arthritis (RA) in a subject. In one embodiment, a method comprises (a) obtaining a biological sample from the subject; (b) determining expression levels of at least two genes in the biological sample; and (c) comparing the expression levels of each of the at least two genes determined in step (b) with a standard, wherein the comparing detects the predisposition to developing established rheumatoid arthritis in the subject. In one embodiment, the biological sample is a cell. In one embodiment, the cell is a peripheral blood mononuclear cell. In one embodiment, the subject is an animal. In one embodiment, the animal is a mammal. In one embodiment, the mammal is a human. In one embodiment, the determining comprises a technique selected from the group consisting of a Northern blot, hybridization to a nucleic acid microarray, and a reverse transcription-polymerase chain reaction (RT-PCR). In one embodiment, the RT-PCR is quantitative RT-PCR.

In one embodiment of the present method, the expression levels of at least two genes represented by SEQ ID NOs 1-94 are determined. In another embodiment, the expression levels of at least five genes represented by SEQ ID NOs: 1-94 are determined. In another embodiment, the expression levels of at least ten genes represented by SEQ ID NOs: 1-94 are determined. In another embodiment, the expression levels of at least twenty genes represented by SEQ ID NOs: 1-94 are determined. In another embodiment, the expression levels of at least twenty-five genes represented by SEQ ID NOs: 1-94 are determined. In still another embodiment, the expression levels of all of the genes represented by SEQ ID NOs: 1-94 are determined.

In one embodiment of the present method, the comparing comprises: (a) establishing an average expression level for each of the at least two genes in a population, wherein the population comprises statistically significant numbers of subjects with early rheumatoid arthritis (RA) and subjects that have established RA; (b) assigning a first value to each gene for which the expression level in the subject is higher than the average expression level in the population and a second value to each gene for which the expression level in the subject is lower than the average expression level in the population; and (c) adding the values assigned in step (b) to arrive at a sum, wherein the sum is indicative of the predisposition of the subject to develop established RA.

The presently claimed subject matter also provides a method for facilitating a diagnosis of rheumatoid arthritis (RA) in a subject, the method comprising (a) providing an array comprising a plurality of nucleic acid sequences, wherein each nucleic acid sequence corresponds to a reference gene; (b) providing a biological sample derived from the subject, wherein the biological sample comprises a nucleic acid; (c) hybridizing the biological sample to the array; (d) detecting all nucleic acids on the array to which the biological sample hybridizes; (e) determining an expression level for each nucleic acid detected; (f) creating a profile of the expression levels for the detected nucleic acids; and (g) comparing the profile created with a standard profile, wherein the comparing facilitates a diagnosis of rheumatoid arthritis (RA) in the subject. In one embodiment, the array is selected from the group consisting of a microarray chip and a membrane-based filter array. In one embodiment, the array comprises nucleic acid sequences corresponding to at least two genes represented by SEQ ID NOs: 1-94. In another embodiment, the array comprises nucleic acid sequences corresponding to at least five genes represented by SEQ ID NOs: 1-94. In another embodiment, the array comprises nucleic acid sequences corresponding to at least ten genes represented by SEQ ID NOs: 1-94. In another embodiment, the array comprises nucleic acid sequences corresponding to at least twenty genes represented by SEQ ID NOs: 1-94. In another embodiment, the array comprises nucleic acid sequences corresponding to at least twenty-five genes represented by SEQ ID NOs: 1-94. In still another embodiment, the array comprises nucleic acid sequences corresponding to all of the genes represented by SEQ ID NOs: 1-94. In one embodiment, the array further comprises nucleic acid sequences corresponding to at least one internal control gene. In one embodiment, the biological sample is a cell. In one embodiment, the cell is a peripheral blood mononuclear cell. In one embodiment, the subject is an animal. In one embodiment, the animal is a mammal. In one embodiment, the mammal is a human.

In one embodiment of the present method, the expression level of a gene is determined using a technique selected from the group consisting of a Northern blot, hybridization to a nucleic acid microarray, and a reverse transcription-polymerase chain reaction (RT-PCR). In one embodiment, the RT-PCR is quantitative RT-PCR.

In one embodiment of the present method, the expression levels of at least two genes represented by SEQ ID NOs: 1-94 are determined. In another embodiment, the expression levels of at least five genes represented by SEQ ID NOs: 1-94 are determined. In another embodiment, the expression levels of the eight genes represented by SEQ ID NOs: 2-9 are determined. In another embodiment, the expression levels of at least ten genes represented by SEQ ID NOs: 1-94 are determined. In another embodiment, the expression levels of the ten genes represented by SEQ ID NOs: 17, 19, 20, 22, 26, 35, 37-39, and 47 are determined. In yet another embodiment, the expression levels of all of the genes represented by SEQ ID NOs: 1-94.

In one embodiment of the present method, the determining an expression level for each nucleic acid detected further comprises normalizing the expression level that is determined for each nucleic acid detected relative to an expression level of another gene present on the array, wherein the another gene present on the array is a gene for which the expression level does not vary in the population.

In one embodiment of the present method, the comparing comprises (a) establishing an average expression level for each gene in a population, wherein the population comprises statistically significant numbers of subjects with early rheumatoid arthritis (RA) and subjects that have established RA; (b) assigning a first value to each gene for which the expression level in the subject is higher than the average expression level in the population and a second value to each gene for which the expression level in the subject is lower than the average expression level in the population; and (c) adding the values assigned in step (b) to arrive at a sum, wherein the sum is indicative of the predisposition of the subject to develop established RA.

In one embodiment, the presently claimed subject matter also provides a kit comprising a plurality of oligonucleotide primers and instructions for employing the plurality of oligonucleotide primers to determine the expression level of at least one of the genes represented by SEQ ID NOs: 1-94. In another embodiment, the kit comprises oligonucleotide primers to determine the expression level of at least five of the genes represented by SEQ ID NOs: 1-94. In another embodiment, the kit comprises oligonucleotide primers to determine the expression level of at least ten of the genes represented by SEQ ID NOs: 1-94. In another embodiment, the kit comprises oligonucleotide primers to determine the expression level of at least twenty of the genes represented by SEQ ID NOs: 1-94. In another embodiment, the kit comprises oligonucleotide primers to determine the expression level of at least thirty of the genes represented by SEQ ID NOs: 1-94. In another embodiment, the kit comprises oligonucleotide primers to determine the expression level of at all of the genes represented by SEQ ID NOs: 1-94. In still another embodiment, the kit further comprises oligonucleotide primers to determine the expression level of a control gene.

### BRIEF DESCRIPTION OF THE DRAWINGS

FIG. 1 depicts the results of clustering with the self-organizing map algorithm on genes filtered for 3 standard deviations (SD) of variability, revealing almost complete separation of the early RA patients from the established RA patients.

FIG. 2 depicts the results of applying a hierarchical clustering algorithm to gene expression data derived from RA patients, which separated the patients into two main clusters. One cluster contained 7 of the 8 established RA patients. The other cluster included all of the early RA patients, including early patient RA9, as well as patient RA8, who had longstanding disease.

FIG. 3 depicts the results of applying a K-means clustering algorithm to the gene expression data. This algorithm showed less definite separation of the two RA subgroups.

FIG. 4 depicts the results of applying two equations to the expression data. The first equation, Equation 1, used 10 genes that were upregulated by at least 4-fold in patients with established RA compared to early RA (see Table 1). The second equation, Equation 2, used 8 genes that were upregulated by at least 3-fold in the early RA patients (see Table 2). Each of these gene equations allowed for the classification of subjects in the two groups with a high degree of accuracy.

### BRIEF DESCRIPTION OF THE SEQUENCE LISTING

SEQ ID NOs: 1 and 2 are the nucleic acid sequences of a partial cDNA and a full-length cDNA, respectively, corresponding to the human cleavage stimulation factor subunit 2 (CSTF2) gene (GenBank accession numbers AA293218 and NM_{—}001325).

SEQ ID NOs: 3 and 4 are the nucleic acid sequences of a partial cDNA and the full-length cDNA, respectively, corresponding to the human colony stimulating factor 3 receptor (granulocyte; CSF3R) gene (GenBank accession numbers AA458507 and NM_{—}156039).

SEQ ID NOs: 5 and 6 are the nucleic acid sequences of a partial cDNA and a full-length cDNA, respectively, corresponding to the human transforming growth factor β receptor II (TGFBR2) gene (GenBank accession numbers AA487034 and D50683).

SEQ ID NOs: 7 and 8 are the nucleic acid sequences of a partial cDNA and a full-length cDNA, respectively, corresponding to the human cytochrome P450 subfamily 3A4 (CYP3A4) gene (GenBank accession numbers R91078 and NM_{—}017460).

SEQ ID NOs: 9 and 10 are the nucleic acid sequences of a partial cDNA and a full-length cDNA, respectively, corresponding to the human 11-β hydroxysteroid dehydrogenase 2 (HSD11B2) gene (GenBank accession numbers W95083 and NM_{—}000196).

SEQ ID NOs: 11 and 12 are the nucleic acid sequences of a partial cDNA and a full-length cDNA, respectively, corresponding to the human troponin I, skeletal, fast twitch (TNNI2) gene (GenBank accession numbers AA181334 and NM_{—}003282).

SEQ ID NOs: 13 and 14 are the nucleic acid sequences of a partial cDNA and a full-length cDNA, respectively, corresponding to the human syntrophin α, neuromuscular junction protein (SNTA1) gene (GenBank accession numbers AA699926 and NM_{—}003098).

SEQ ID NOs: 15 and 16 are the nucleic acid sequences of a partial cDNA and a full-length cDNA, respectively, corresponding to the human cardiac troponin T2 (TNNT2) gene (GenBank accession numbers N70734 and NM_{—}000364).

SEQ ID NOs: 17 and 18 are the nucleic acid sequences of a partial cDNA and a full-length cDNA, respectively, corresponding to the human zinc finger protein 74 (Cos52; ZNF74) gene (GenBank accession numbers AA629838 and NM_{—}003426).

SEQ ID NOs: 19 and 20 are the nucleic acid sequences of a partial cDNA and a full-length cDNA, respectively, corresponding to the human chemokine receptor (c-c motif; CR1) gene (GenBank accession numbers AA036881 and NM_{—}001295).

SEQ ID NOs: 21 and 22 are the nucleic acid sequences of a partial cDNA, and a full-length cDNA, respectively, corresponding to the human prostaglandin E synthase (PTGES) gene (GenBank accession numbers AA436163 and NM_{—}004878).

SEQ ID NOs: 23 and 24 are the nucleic acid sequences of a partial cDNA and a full-length cDNA, respectively, corresponding to the human mannose-binding lectin 1 (LMAN1) gene (GenBank accession numbers AA446103 and NM_{—}005570).

SEQ ID NOs: 25 and 26 are the nucleic acid sequences of a partial cDNA and a full-length cDNA, respectively, corresponding to the human nuclease sensitive element binding protein (NSEP1) gene (GenBank accession numbers AA599175 and NM_{—}004559).

SEQ ID NOs: 27 and 28 are the nucleic acid sequences of a partial cDNA and a full-length cDNA, respectively, corresponding to the human FK506 binding protein 1A (FKBP1A) gene (GenBank accession numbers AA625981 and NM_{—}000801).

SEQ ID NOs: 29 and 30 are the nucleic acid sequences of a partial cDNA and a full-length cDNA, respectively, corresponding to the human interferon gamma-inducible protein 30 (IFI30) gene (GenBank accession numbers AA630800 and NM_{—}006332).

SEQ ID NOs: 31 and 32 are are the nucleic acid sequences of a partial cDNA and a full-length cDNA, respectively, corresponding to the human MHC class II DP α1 (HLA-DPA1) gene (GenBank accession numbers AA634028 and NM_{—}033554).

SEQ ID NOs: 33 and 34 are the nucleic acid sequences of a partial cDNA and a full-length cDNA, respectively, corresponding to the human β₂-microglobulin (B2M) gene (GenBank accession numbers AA670408 and NM_{—}004048).

SEQ ID NOs: 35 and 36 are the nucleic acid sequences of a partial cDNA and a full-length cDNA, respectively, corresponding to the human FYN-binding protein (FYB) gene (GenBank accession numbers N64862 and NM_{—}001465).

SEQ ID NOs: 37 and 38 are the nucleic acid sequences of a partial cDNA and a full-length cDNA, respectively, corresponding to the human MHC class II DR α (HLA-DRA) gene (GenBank accession numbers R47979 and NM_{—}019111).

SEQ ID NOs: 39 and 40 are the nucleic acid sequences of a partial cDNA and a full-length cDNA, respectively, corresponding to the human spermidine/spermine N1-acetyltransferase (SAT) gene (GenBank accession numbers AA011215 and NM_{—}002970).

SEQ ID NOs: 41 and 42 are the nucleic acid sequences of a partial cDNA and a full-length cDNA, respectively, corresponding to the human RAS oncogene family (RAB7) gene (GenBank accession numbers AA496780 and NM_{—}004637).

SEQ ID NOs: 43 and 44 are the nucleic acid sequences of a partial cDNA and a full-length cDNA, respectively, corresponding to the human synovial sarcoma breakpoint 3 (SSX3) gene (GenBank accession numbers AA609599 and NM_{—}021014).

SEQ ID NOs: 45 and 46 are the nucleic acid sequences of a partial cDNA and a full-length cDNA, respectively, corresponding to the human 67 kD laminin receptor 1 (LAMR1) gene (GenBank accession numbers AA629897 and NM_{—}002295).

SEQ ID NOs: 47 and 48 are the nucleic acid sequences of a partial cDNA and a full-length cDNA, respectively, corresponding to the human ovarian carcinoma antigen (CA-125; M17S2) gene (GenBank accession numbers AA676470 and NM_{—}031862).

SEQ ID NOs: 49 and 50 are the nucleic acid sequences of a partial cDNA and a full-length cDNA, respectively, corresponding to the human protein tyrosine phosphatase (PTPRA) gene (GenBank accession numbers H82419 and NM_{—}002836).

SEQ ID NOs: 51 and 52 are the nucleic acid sequences of a partial cDNA and a full-length cDNA, respectively, corresponding to the human sarcoma amplified sequence (SAS) gene (GenBank accession numbers R45413 and NM_{—}005981).

SEQ ID NOs: 53 and 54 are the nucleic acid sequences of a partial cDNA and a full-length cDNA, respectively, corresponding to the human L-plastin (LCP-1) gene (GenBank accession numbers W73144 and NM_{—}002298).

SEQ ID NOs: 55 and 56 are the nucleic acid sequences of a partial cDNA and a full-length cDNA, respectively, corresponding to the human LIM and SHE protein-1 (LASP-1) gene (GenBank accession numbers W80637 and NM_{—}006148).

SEQ ID NOs: 57 and 58 are the nucleic acid sequences of a partial cDNA and a full-length cDNA, respectively, corresponding to the human sorting nexin 2 (SNX2) gene (GenBank accession numbers AA171463 and NM_{—}003100).

SEQ ID NOs: 59 and 60 are the nucleic acid sequences of a partial cDNA and a full-length cDNA, respectively, corresponding to the human EGF-response factor 1 (ZFP36L1) gene (GenBank accession numbers AA424743 and NM_{—}004926).

SEQ ID NOs: 61 and 62 are the nucleic acid sequences of a partial cDNA and a full-length cDNA, respectively, corresponding to the human latent TGF-β binding protein 1 (LTBP1) gene (GenBank accession numbers AA490011 and NM_{—}000627).

SEQ ID NOs: 63 and 64 are the nucleic acid sequences of a partial cDNA, and a full-length cDNA, respectively, corresponding to the human ornithine decarboxylase Antizyme 1 (OAZ1) gene (GenBank accession numbers AA487466 and NM_{—}004152).

SEQ ID NOs: 65 and 66 are the nucleic acid sequences of a partial cDNA and a full-length cDNA, respectively, corresponding to the human cytochrome P450 subfamily 24 (CYP24A1) gene (GenBank accession numbers N21576 and NM_{—}000782).

SEQ ID NOs: 67 and 68 are the nucleic acid sequences of a partial cDNA and a full-length cDNA, respectively, corresponding to the human cytochrome P450 oxidoreductase (POR) gene (GenBank accession numbers T73294 and NM_{—}000941).

SEQ ID NOs: 69 and 70 are the nucleic acid sequences of a partial cDNA and a full-length cDNA, respectively, corresponding to the human chitinase 3-like 1 (cartilage glycoprotein-39; CHI3L1) gene (GenBank accession numbers AA434115 and NM_{—}001276).

SEQ ID NOs: 71 and 72 are the nucleic acid sequences of a partial cDNA and a full-length cDNA, respectively, corresponding to the human bone morphogenic protein 4 (BMP4) gene (GenBank accession numbers AA463225 and NM_{—}001202).

SEQ ID NOs: 73 and 74 are the nucleic acid sequences of a partial cDNA and a full-length cDNA, respectively, corresponding to the human regulator of G-protein signaling 4 (RGS4) gene (GenBank accession numbers AA007419 and BC051869).

SEQ ID NOs: 75 and 76 are the nucleic acid sequences of a partial cDNA and a full-length cDNA, respectively, corresponding to the human hemoglobin zeta (HBZ) gene (GenBank accession numbers N59636 and NM_{—}005332).

SEQ ID NOs: 77 and 78 are the nucleic acid sequences of a partial cDNA and a full-length cDNA, respectively, corresponding to the human translation elongation factor 2 (EEF2) gene (GenBank accession numbers R43766 and NM_{—}001961).

SEQ ID NOs: 79 and 80 are the nucleic acid sequences of a partial cDNA and a full-length cDNA, respectively, corresponding to the human adenylyl cyclase-associated protein (CAP) gene (GenBank accession numbers R37953 and NM_{—}006367).

SEQ ID NOs: 81 and 82 are the nucleic acid sequences of a partial cDNA and a full-length cDNA, respectively, corresponding to the human calpactin 1 (S100A10) gene (GenBank accession numbers AA444051 and NM_{—}002966).

SEQ ID NOs: 83 and 84 are the nucleic acid sequences of a partial cDNA and a full-length cDNA, respectively, corresponding to the human actin-related protein subunit 5 (ARPC5) gene (GenBank accession numbers W55964 and NM_{—}005717).

SEQ ID NOs: 85 and 86 are the nucleic acid sequences of a partial cDNA and a full-length cDNA, respectively, corresponding to the human rho GDP dissociation inhibitor (ARHGDIB) gene (GenBank accession numbers AA487426 and NM_{—}001175).

SEQ ID NOs: 87 and 88 are the nucleic acid sequences of a partial cDNA and a full-length cDNA, respectively, corresponding to the human methyltransferase-like 1 (METTL1) gene (GenBank accession numbers AA422058 and NM_{—}005371).

SEQ ID NOs: 89 and 90 are the nucleic acid sequences of a partial cDNA and a full-length cDNA, respectively, corresponding to the human actin-related protein subunit 3 (ARPC3) gene (GenBank accession numbers H73276 and NM_{—}005719).

SEQ ID NOs: 91 and 92 are the nucleic acid sequences of a partial cDNA and a full-length cDNA, respectively, corresponding to the human hypoxia inducible factor (HIF1A) gene (GenBank accession numbers AA598526 and NM_{—}001530).

SEQ ID NOs: 93 and 94 are the nucleic acid sequences of a partial cDNA and a full-length cDNA, respectively, corresponding to the human checkpoint suppressor 1 (CHES1) gene (GenBank accession numbers H84982 and NM_{—}005197).

### DETAILED DESCRIPTION

Disclosed is a method for detecting a predisposition in a subject to develop established rheumatoid arthritis (RA) by analyzing gene expression profiles for selected genes in biological samples isolated from the subject and comparing the gene expression profiles to standards. In one embodiment, the method involves determining the expression levels of a set of genes expressed in peripheral blood mononuclear cells isolated from a subject suspected of having RA and comparing the expression levels of these genes with the levels of expression of these genes in subjects with confirmed early and established RA. Using the method, it is possible to determine whether or not the subject is likely to develop established RA.

In determining whether or not a subject has a predisposition to developing established RA, the expression levels of many genes can be analyzed simultaneously using microarrays or membrane-based filter arrays. A representative filter array is the GF211 Human “Named Genes” GENEFILTERS® Microarrays Release 1 (available from RESGEN™, a division of Invitrogen Corporation, Carlsbad, Calif., United States of America), although other arrays can also be used. Using the GF211 array, it is possible to determine the expression levels of over 4000 genes simultaneously in a biological sample. Additionally, the presence on the GF211 filter of certain “housekeeping” genes allows for the comparison of data from experiment to experiment. This facilitates the comparison of newly obtained data to a standard (e.g. a previously generated standard).

I. Definitions

While the following terms are believed to be well understood by one of ordinary skill in the art, the following definitions are set forth to facilitate explanation of the claimed subject matter.

Following long-standing patent law convention, the terms “a” and “an” mean “one or more” when used in this application, including the claims.

As used herein, the term “about,” when referring to a value or to an amount of mass, weight, time, volume, concentration or percentage is meant to encompass variations of ±20% or ±10%, in another example ±5%, in another example ±1%, and in still another example ±0.1% from the specified amount, as such variations are appropriate to perform the disclosed method.

As used herein, “significance” or “significant” relates to a statistical analysis of the probability that there is a non-random association between two or more entities. To determine whether or not a relationship is “significant” or has “significance”, statistical manipulations of the data can be performed to calculate a probability, expressed as a “p-value”. Those p-values that fall below a user-defined cutoff point are regarded as significant. In one example, a p-value less than or equal to 0.05, in another example less than 0.01, in another example less than 0.005, and in yet another example less than 0.001, are regarded as significant.

I.A. Nucleic Acids

The nucleic acid molecules employed in accordance with the presently claimed subject matter include any nucleic acid molecule for which expression is desired to be assessed in evaluating the presence or absence of an autoimmune disease, in one embodiment, rheumatoid arthritis. Representative nucleic acid molecules include, but are not limited to the isolated nucleic acid molecules of any one of SEQ ID NOs: 1-94, complementary DNA molecules, sequences having at least 80% identity as disclosed herein to any one of SEQ ID NOs: 1-94, sequences capable of hybridizing to any one of SEQ ID NOs: 1-94 under conditions disclosed herein, and corresponding RNA molecules.

As used herein, “nucleic acid” and “nucleic acid molecule” refer to any of deoxyribonucleic acid (DNA), ribonucleic acid (RNA), oligonucleotides, fragments generated by the polymerase chain reaction (PCR), and fragments generated by any of ligation, scission, endonuclease action, and exonuclease action. Nucleic acids can comprise monomers that are naturally occurring nucleotides (such as deoxyribonucleotides and ribonucleotides), or analogs of naturally occurring nucleotides (e.g., α-enantiomeric forms of naturally occurring nucleotides), or a combination of both. Modified nucleotides can have modifications in sugar moieties and/or in pyrimidine or purine base moieties. Sugar modifications include, for example, replacement of one or more hydroxyl groups with halogens, alkyl groups, amines, and azido groups. Sugars can also be functionalized as ethers or esters. Moreover, the entire sugar moiety can be replaced with sterically and electronically similar structures, such as aza-sugars and carbocyclic sugar analogs. Examples of modifications in a base moiety include alkylated purines and pyrimidines, acylated purines or pyrimidines, or other well-known heterocyclic substitutes. Nucleic acid monomers can be linked by phosphodiester bonds or analogs of phosphodiester bonds. Analogs of phosphodiester linkages include phosphorothioate, phosphorodithioate, phosphoroselenoate, phosphorodiselenoate, phosphoroanilothioate, phosphoranilidate, phosphoramidate, and the like.

Unless otherwise indicated, a particular nucleotide sequence also implicitly encompasses complementary sequences, subsequences, elongated sequences, as well as the sequence explicitly indicated. The terms “nucleic acid molecule” or “nucleotide sequence” can also be used in place of “gene”, “cDNA”, or “mRNA”. Nucleic acids can be derived from any source, including any organism. In one embodiment, a nucleic acid is derived from a biological sample isolated from a subject.

The term “subsequence” refers to a sequence of nucleic acids that comprises a part of a longer nucleic acid sequence. An exemplary subsequence is a probe, or a primer. The term “primer” as used herein refers to a contiguous sequence comprising in one example about 8 or more deoxyribonucleotides or ribonucleotides, in another example 10-20 nucleotides, and in yet another example 20-30 nucleotides of a selected nucleic acid molecule. The primers disclosed herein encompass oligonucleotides of sufficient length and appropriate sequence so as to provide initiation of polymerization on a target nucleic acid molecule.

The term “elongated sequence” refers to an addition of nucleotides (or other analogous molecules) incorporated into the nucleic acid. For example, a polymerase (e.g., a DNA polymerase) can add sequences at the 3′ terminus of the nucleic acid molecule. In addition, the nucleotide sequence can be combined with other DNA sequences, such as promoters, promoter regioris, enhancers, polyadenylation signals, intronic sequences, additional restriction enzyme sites, multiple cloning sites, and other coding segments.

As used herein, the phrases “open reading frame” and “ORF” are given their common meaning and refer to a contiguous series of deoxyribonucleotides or ribonucleotides that encode a polypeptide or a fragment of a polypeptide. In an organism that splices precursor RNAs to form in RNAs, the ORF will be discontinuous in the genome. Splicing produces a continuous ORF that can be translated to produce a polypeptide. In a full-length cDNA, the complete ORF includes, those nucleic acid sequences beginning with the start codon and ending with the stop codon. In a cDNA molecule that is not full-length, the ORF includes those nucleic acid sequences present in the non-full-length cDNA that are included within the complete ORF of the corresponding full-length cDNA.

As used herein, the phrase “coding sequence” is used interchangeably with “open reading frame” and “ORF” and refers to a nucleic acid sequence that is transcribed into RNA including, but not limited to mRNA, rRNA, tRNA, snRNA, sense RNA, or antiserise RNA. The RNA can then be translated in vitro or in vivo to produce a protein.

The terms “complementary” and “complementary sequences”, as used herein, refer to two nucleotide sequences that comprise antiparallel nucleotide sequences capable of pairing with one another upon formation of hydrogen bonds between base pairs. As used herein, the term “complementary sequences” means nucleotide sequences which are substantially complementary, as can be assessed by the same nucleotide comparison set forth herein, or is defined as being capable of hybridizing to the nucleic acid segment in question under relatively stringent conditions such as those described herein. In one embodiment, a complementary sequence is at least 80% complementary to the nucleotide sequence with which is it capable of pairing. In another embodiment, a complementary sequence is at least 85% complementary to the nucleotide sequence with which is it capable of pairing. In another embodiment, a complementary sequence is at least 90% complementary to the nucleotide sequence with which is it capable of pairing. In another embodiment, a complementary sequence is at least 95% complementary to the nucleotide sequence with which is it capable of pairing. In another embodiment, a complementary sequence is at least 98% complementary to the nucleotide sequence with which is it capable of pairing. In another embodiment, a complementary sequence is at least 99% complementary to the nucleotide sequence with which is it capable of pairing. In still another embodiment, a complementary sequence is at 100% complementary to the nucleotide sequence with which is it capable of pairing. A particular example of a complementary nucleic acid segment is an antisense oligonucleotide.

The term “gene” refers broadly to any segment of DNA associated with a biological function. A gene encompasses sequences including, but not limited to a coding sequence, a promoter region, a transcriptional regulatory sequence, a non-expressed DNA segment that is a specific recognition sequence for regulatory proteins, a non-expressed DNA segment that contributes to gene expression, a DNA segment designed to have desired parameters, or combinations thereof. A gene can be obtained by a variety of methods, including isolation or cloning from a biological sample, synthesis based on known or predicted sequence information, and recombinant derivation of an existing sequence.

As used herein, the terms “known gene” and “reference gene” are used interchangeably and refer to nucleic acid sequences that can be identified as corresponding to a particular expressed sequence tag (EST), partial cDNA, full-length cDNA, or gene. In one embodiment, a reference gene is a gene, cDNA, or an EST for which the nucleic acid sequence has been determined (i.e. is known). In another embodiment, a reference gene is represented by one of the nucleic acid sequences disclosed in SEQ ID NOs: 1-94. In another embodiment, a reference gene is represented by a nucleic acid sequence complementary to one of the nucleic acid sequences disclosed in SEQ ID NOs: 1-94. In another embodiment, a reference gene is represented by a nucleic acid sequence having 80% identity to any one of SEQ ID NOs: 1-94. In another embodiment, a reference gene is represented by a nucleic acid sequence capable of hybridizing to any one of SEQ ID NOs: 1-94 under conditions disclosed herein. In another embodiment, a reference gene is represented by an RNA molecule corresponding to any one of SEQ ID NOs: 1-94. In another embodiment, a reference gene is represented by a nucleic acid sequence present on an array.

As used herein, the terms “corresponding to” and “representing”, “represented by” and grammatical derivatives thereof, when used in the context of a nucleic acid sequence corresponding to or representing a gene, refers to a nucleic acid sequence that results from transcription, reverse transcription, or replication from a particular genetic locus, gene, or gene product (for example, an mRNA). In other words, an EST, partial cDNA, or full-length cDNA corresponding to a particular reference gene is a nucleic acid sequence that one of ordinary skill in the art would recognize as being a product of either transcription or replication of that reference gene (for example, a product produced by transcription of the reference gene). One of ordinary skill in the art would understand that the EST, partial cDNA, or full-length cDNA itself is produced by in vitro manipulation to convert the mRNA into an EST or cDNA, for example by reverse transcription of an isolated RNA molecule that was transcribed from the reference gene. One of ordinary skill in the art will also understand that the product of a reverse transcription is a double-stranded DNA molecule, and that a given strand of that double-stranded molecule can embody either the coding strand or the non-coding strand of the gene. The sequences presented in the Sequence Listing are single-stranded, however, and it is to be understood that the presently claimed subject matter is intended to encompass the genes represented by the sequences presented in SEQ ID NOs: 1-94, including the specific sequences set forth as well as the reverse/complement of each of these sequences.

A known gene and/or reference gene also includes, but is not limited to those genes that have been identified as being differentially expressed in early RA patients versus established RA patients, such as but not limited to those set forth in Tables 4 and 5. A reference gene is also intended to include nucleic acid sequences that substantially hybridize to one of such genes, including but not limited to one of the nucleic acid sequences disclosed in SEQ ID NOs: 1-94. As such, a reference gene includes a nucleic acid sequence that has one or more polymorphisms such that while the particular nucleic acid sequence might diverge somewhat from one of such genes, including but not limited to one of those disclosed in SEQ ID NOs: 1-94, one of ordinary skill in the art would nonetheless recognize the particular nucleic acid sequence as corresponding to a gene represented by one of such genes, including but not limited to one of the sequences disclosed in SEQ ID NOs: 1-94. For example, the GenBank database has at least four accession numbers that are identified as corresponding to the human colony stimulating factor 3 receptor (CSF3R) mRNA. These four represent transcript variants 1-4, and have accession numbers NM_{—}000760, NM_{—}156038, NM_{—}156039, NM_{—}172313, respectively. It is understood that the presently claimed subject matter, which identifies NM_{—}156039 as SEQ ID NO: 4, also encompasses the other transcript variants.

As used herein, the term “early RA” refers to an early state in the development of rheumatoid arthritis characterized by early synovitis but without the appearance of extensive erosive joint disease. As a proxy for patients that are in the early stages of RA, an early RA patient is also defined as a subject that has had a diagnosis of RA for less than two years.

Not all patients who suffer from early synovitis go on to develop established RA. The term “established RA” as used herein refers to a disease state in which the diagnostic criteria for RA (Arnett et al., 1988) have been present for more than 2 years. Early RA patients, on the other hand, might or might not satisfy the diagnostic criteria, and have had symptoms or findings for 2 years or less.

The term “gene expression” generally refers to the cellular processes by which a biologically active polypeptide is produced from a DNA sequence. Generally, gene expression comprises the processes of transcription and translation, along with those modifications that normally occur in the cell to modify the newly translated protein to an active form and to direct it to its proper subcellular or extracellular location.

The terms “gene expression level” and “expression level” as used herein refer to an amount of gene-specific RNA or polypeptide that is present in a biological sample. When used in relation to an RNA molecule, the term “abundance” can be used interchangeably with the terms “gene expression level” and “expression level”. While an expression level can be expressed in standard units such as “transcripts per cell” for RNA or “nanograms per microgram tissue” for RNA or a polypeptide, it is not necessary that expression level be defined as such. Alternatively, relative units can be employed to describe an expression level. For example, when the assay has an internal control (referred to herein as a “control gene”, which is in one embodiment), which can be, for example, a known quantity of a nucleic acid derived from a gene for which the expression level is either known or can be accurately determined, unknown expression levels of other genes can be compared to the known internal control. More specifically, when the assay involves hybridizing labeled total RNA to a solid support comprising a known amount of nucleic acid derived from reference genes, an appropriate internal control could be a housekeeping gene (e.g. glucose-6-phosphate dehydrogenase or elongation factor-1), a housekeeping gene being defined as a gene for which the expression level in all cell types and under all conditions is substantially the same. Use of such an internal control allows a discrete expression level for a gene to be determined (e.g. relative to the expression of the housekeeping gene) both for the nucleic acids present on the solid support and also between different experiments using the same solid support. This discrete expression level can then be normalized to a value relative to the expression level of the control gene (for example, a housekeeping gene).

As used herein, the term “normalized”, and grammatical derivatives thereof, refers to a manipulation of discrete expression level data wherein the expression level of a reference gene is expressed relative to the expression level of a control gene. For example, the expression level of the control gene can be set at 1, and the expression levels of all reference genes can be expressed in units relative to the expression of the control gene.

The term “average expression level” as used herein refers to the mean expression level, in whatever units are chosen, of a gene in a particular biological sample of a population. To determine an average expression level, a population is defined, and the expression level of the gene in that population is determined for each member of the population by analyzing the same biological sample from each member of the population. The determined expression levels are then added together, and the sum is divided by the number of members in the population.

The term “average expression level” is also used to refer to a calculated value that can be used to compare two populations. For example, the average expression level in a population consisting of all RA patients regardless of their classifications as early or established can be calculated using the method above for a population that consists of statistically significant numbers of early RA and established RA patients. However, when the population is made up of unequal numbers of early and established RA patients, the calculated value for all genes differentially expressed in these two subpopulations will likely be skewed towards the expression level determined for the subpopulation having the greater number of members. In order to remove this skewing effect, the average expression level in the RA population can also be calculated by: (a) determining the average expression level of a gene in the early RA subpopulation; (b) determining the average expression level of the same gene in the established RA sub population; (c) adding the two determined values together; and (d) dividing the sum of the two determined values by 2 to achieve a value: this value also being defined herein as an “average expression level”.

Once an expression level is determined for a gene, a profile can be created. As used herein, the term “profile” refers to a repository of the expression level data that can be used to compare the expression levels of different genes among various subjects. For example, for a given subject, the term “profile” can encompass the expression levels of all genes detected in whatever units (as described herein above) that are chosen.

The term “profile” is also intended to encompass manipulations of the expression level data derived from a subject. For example, once relative expression levels are determined for a given set of genes in a subject, the relative expression levels for that subject can be compared to a standard to determine if the expression levels in that subject are higher or lower than for the same genes in the standard. Standards can include any data deemed to be relevant for comparison. In one embodiment, a standard is prepared by determining the average expression level of a gene in a population of patients with early RA. In another embodiment, a standard is prepared by determining the average expression level of a gene in a population of subjects that have established RA. In a third embodiment, a standard is prepared by determining the average expression level of a gene in the population as a whole (i.e. RA patients are grouped together irrespective of the duration of their disease).

In yet another embodiment, a standard is prepared by determining the average expression level of a gene in the early RA population, the average expression level of a gene in the established RA population, adding those two values, and dividing the sum by two to determine the midpoint of the average expression in these populations. In this latter embodiment, a profile for a “new” subject can be compared to the standard, and the profile can further comprise data indicating whether for each gene, the expression level in the new subject is higher or lower than the expression level of that gene in the standard. For example, a new subject's profile can comprise a score or value of “1” for each gene for which the expression in the subject is higher than in the standard, and a score or value of “0” for each gene for which the expression in the subject is lower than in the standard. In this way, a profile can comprise an overall “score”, the score being defined as the sum total of all the 1s and 0s present in the profile when Equation 1 or Equation 2 is applied to the data in the profile. These scores can then be used to detect a predisposition to developing established RA in the new subject. It is understood that the use of 1s and 0s is exemplary only, and any convenient value can be assigned in the practice of the methods of the presently claimed subject matter.

The term “predisposition” as used herein refers to a likelihood that a subject will develop established RA absent treatment to reverse the progress of the disease. As such, “a predisposition to developing established RA” refers to a state of health wherein a subject's body has undergone biochemical changes that without medical intervention will lead to the development of the established form of RA.

The phrases “percent identity” and “percent identical,” in the context of two nucleic acid or protein sequences, refer to two or more sequences or subsequences that have in one embodiment at least 60%, in another embodiment at least 70%, in another embodiment at least 80%, in another embodiment at least 85%, in another embodiment at least 90%, in another embodiment at least 95%, in another embodiment at least 98%, and in yet another embodiment at least 99% nucleotide or amino acid residue identity, when compared and aligned for maximum correspondence, as measured using one of the following sequence comparison algorithms or by visual inspection. The percent identity exists in one embodiment over a region of the sequences that is at least about 50 residues in length, in another embodiment over a region of at least about 100 residues, and in still another embodiment the percent identity exists over at least about 150 residues. In yet another embodiment, the percent identity exists over the entire length of a given region, such as a coding region. In one embodiment, a nucleic acid is at least 80% identical to one of SEQ ID NOs: 1-94.

For sequence comparison, typically one sequence acts as a reference sequence to which test sequences are compared. When using a sequence comparison algorithm, test and reference sequences are input into a computer, subsequence coordinates are designated if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, for example, by the local homology algorithm described in Smith & Waterman, 1981, by the homology alignment algorithm described in Needleman & Wunsch, 1970, by the search for similarity method described in Pearson & Lipman, 1988, by computerized implementations of these algorithms (GAP, BESTFIT, FASTA, and TFASTA in the GCG Wisconsin Package, available from Accelrys, Inc., San Diego, Calif., United States of America), or by visual inspection. See generally, Ausubel et al., 1994.

One example of an algorithm that is suitable for determining percent sequence identity and sequence similarity is the BLAST algorithm, which is described in Altschul et al., 1990. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., 1990). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always>0) and N (penalty score for mismatching residues; always<0). For amino acid sequences, a scoring matrix is used to calculate the cumulative score. Extension of the word hits in each direction are halted when the cumulative alignment score falls off by the quantity X from its maximum achieved value, the cumulative score goes to zero or below due to the accumulation of one or more negative-scoring residue alignments, or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, a cutoff of 100, M=5, N=−4, and a comparison of both strands. For amino acid sequences, the BLASTP program uses as defaults a wordlength (W) of 3, an expectation (E) of 10, and the BLOSUM62 scoring matrix. See Henikoff & Henikoff, 1989.

In addition to calculating percent sequence identity, the BLAST algorithm also performs a statistical analysis of the similarity between two sequences. See e.g., Karlin & Altschul, 1993. One measure of similarity provided by the BLAST algorithm is the smallest sum probability (P(N)), which provides an indication of the probability by which a match between two nucleotide or amino acid sequences would occur by chance. For example, a test nucleic acid sequence is considered similar to a reference sequence if the smallest sum probability in a comparison of the test nucleic acid sequence to the reference nucleic acid sequence is in one embodiment less than about 0.1, in another embodiment less than about 0.01, and in still another embodiment less than about 0.001.

The term “substantially identical”, in the context of two nucleotide sequences, refers to two or more sequences or subsequences that have in one embodiment at least about 80% nucleotide identity, in another embodiment at least about 85% nucleotide identity, in another embodiment at least about 90% nucleotide identity, in another embodiment at least about 95% nucleotide identity, in another embodiment at least about 98% nucleotide identity, and in yet another embodiment at least about 99% nucleotide identity, when compared and aligned for maximum correspondence, as measured using one of the following sequence comparison algorithms or by visual inspection. In one example, the substantial identity exists in nucleotide sequences of at least 50 residues, in another example in nucleotide sequence of at least about 100 residues, in another example in nucleotide sequences of at least about 150 residues, and in yet another example in nucleotide sequences comprising complete coding sequences. In one aspect, polymorphic sequences can be substantially identical sequences. The term “polymorphic” refers to the occurrence of two or more genetically determined alternative sequences or alleles in a population. An allelic difference can be as small as one base pair. Nonetheless, one of ordinary skill in the art would recognize that the polymorphic sequences correspond to the same gene. For example, SEQ ID NO: 33 is an EST derived from the human 2-microglobulin gene. The human β₂-microglobulin complete cDNA is present in the GenBank database under. Accession Number NM_{—}004048, and according to the description presented therein, the β₂-microglobulin gene is characterized by polymorphisms at nucleotide positions 595, 605, and 900. Nucleic acid sequences comprising any or all of these polymorphism are substantially identical to SEQ ID NO: 33, and thus are intended to be encompassed within the claimed subject matter.

Another indication that two nucleotide sequences are substantially identical is that the two molecules specifically or substantially hybridize to each other under stringent conditions. In the context of nucleic acid hybridization, two nucleic acid sequences being compared can be designated a “probe sequence” and a “target sequence”. A “probe sequence” is a reference nucleic acid molecule, and a “target sequence” is a test nucleic acid molecule, often found within a heterogeneous population of nucleic acid molecules. A “target sequence” is synonymous with a “test sequence”.

An exemplary nucleotide sequence employed for hybridization studies or assays includes probe sequences that are complementary to or mimic in one embodiment at least an about 14 to 40 nucleotide sequence of a nucleic acid molecule set forth in SEQ ID NOs: 1-94. In one example, probes comprise 14 to 20 nucleotides, or even longer where desired, such as 30, 40, 50, 60, 100, 200, 300, or 500 nucleotides or up to the full length of any of the genes represented by SEQ ID NOs: 1-94. Such fragments can be readily prepared by, for example, directly synthesizing the fragment by chemical synthesis, by application of nucleic acid amplification technology, or by introducing selected sequences into recombinant vectors for recombinant production. The phrase “hybridizing specifically to” refers to the binding, duplexing, or hybridizing of a molecule only to a particular nucleotide sequence under stringent conditions when that sequence is present in a complex nucleic acid mixture (e.g., total cellular DNA or RNA).

The phrase “hybridizing substantially to” refers to complementary hybridization between a probe nucleic acid molecule and a target nucleic acid molecule and embraces minor mismatches (for example, polymorphisms) that can be accommodated by reducing the stringency of the hybridization media to achieve the desired hybridization.

“Stringent hybridization conditions” and “stringent hybridization wash conditions” in the context of nucleic acid hybridization experiments such as Southern and Northern blot analysis are both sequence- and environment-dependent. Longer sequences hybridize specifically at higher temperatures. An extensive guide to the hybridization of nucleic acids is found in Tijssen, 1993. Generally, highly stringent hybridization and wash conditions are selected to be about 5° C. lower than the thermal melting point (Tₘ) for the specific sequence at a defined ionic strength and pH. Typically, under “stringent conditions” a probe will hybridize specifically to its target subsequence, but to no other sequences.

The Tₘ is the temperature (under defined ionic strength and pH) at which 50% of the target sequence hybridizes to a perfectly matched probe. Very stringent conditions are selected to be equal to the Tₘ for a particular probe. An example of stringent hybridization conditions for Southern or Northern Blot analysis of complementary nucleic acids having more than about 100 complementary residues is overnight hybridization in 50% formamide with 1 mg of heparin at 42° C. An example of highly stringent wash conditions is 15 minutes in 0.1×SSC, SM NaCl at 65° C. An example of stringent wash conditions is 15 minutes in 0.2×SSC buffer at 65° C. (see Sambrook and Russell, 2001, for a description of SSC buffer). Often, a high stringency wash is preceded by a low stringency wash to remove background probe signal. An example of medium stringency wash conditions for a duplex of more than about 100 nucleotides is 15 minutes in 1×SSC at 45° C. An example of low stringency wash for a duplex of more than about 100 nucleotides is 15 minutes in 4-6×SSC at 40° C. For short probes (e.g., about 10 to 50 nucleotides), stringent conditions typically involve salt concentrations of less than about 1 M Na⁺ ion, typically about 0.01 to 1M Na⁺ ion concentration (or other salts) at pH 7.0-8.3, and the temperature is typically at least about 30° C. Stringent conditions can also be achieved with the addition of destabilizing agents such as formamide. In general, a signal to noise ratio of 2-fold (or higher) than that observed for an unrelated probe in the particular hybridization assay indicates detection of a specific hybridization.

The following are examples of hybridization and wash conditions that can be used to clone homologous nucleotide sequences that are substantially identical to reference nucleotide sequences of the presently claimed subject matter: a probe nucleotide sequence hybridizes in one example to a target nucleotide sequence in 7% sodium dodecyl sulfate (SDS), 0.5M NaPO₄, 1 mm EDTA at 50° C. followed by washing in 2×SSC, 0.1% SDS at 50° C.; in another example, a probe and target sequence hybridize in 7% SDS, 0.5M NaPO₄, 1 mm EDTA at 50° C. followed by washing in 1×SSC, 0.1% SDS at 50° C.; in another example, a probe and target sequence hybridize in 7% SDS, 0.5M NaPO₄, 1 mm EDTA at 50° C. followed by washing in 0.5×SSC, 0.1% SDS at 50° C.; in another example, a probe and target sequence hybridize in 7% SDS, 0.5M NaPO₄, 1 mm EDTA at 50° C. followed by washing in 0.1×SSC, 0.1% SDS at 50° C.; in yet another example, a probe and target sequence hybridize in 7% SDS, 0.5M NaPO₄, 1 mm EDTA at 50° C. followed by washing in 0.1×SSC, 0.1% SDS at 65° C. In one embodiment, hybridization conditions comprise hybridization in a roller tube for at least 12 hours at 42° C.

Pre-made hybridization solutions are also commercially available from various suppliers. In one embodiment, a hybridization solution comprises MICROHYB™ (RESGEN™), and in another embodiment a hybridization solution comprises MICROHYB™ further comprising 5.0 μg COT-1® DNA (Invitrogen Corporation, Carlsbad, Calif., United States of America) and 5.0 μg poly-dA. In one embodiment, post-hybridization wash conditions comprise two washes in 2×SSC/1% SDS at 50° C. for 20 minutes each followed by a third wash in 0.5×SSC/1% SDS at 55° C. for 15 minutes.

As used herein, the terms “isolated” and “purified”, when applied to a nucleic acid or protein, are used interchangeably and denote that the nucleic acid or protein is essentially free of other cellular components with which it is associated in the natural state. It can be in a homogeneous state although it also can be in either a dry or aqueous solution. Purity and homogeneity are typically determined using analytical chemistry techniques such as polyacrylamide gel electrophoresis or high performance liquid chromatography. A protein that is the predominant species present in a preparation is substantially purified. The terms “isolated” and “purified” denote that a nucleic acid or protein gives rise to essentially one band in an electrophoretic gel. Particularly, it means that the nucleic acid or protein is in one embodiment at least about 50% pure, in another embodiment at least about 85% pure, and in still another embodiment at least about 99% pure.

I.B. Biological Samples

The presently claimed subject matter provides methods that can be used to detect the expression level of a gene in a biological sample. The term “biological sample” as used herein refers to a sample that comprises a biomolecule that permits the expression level of a gene to be determined. Representative biomolecules include, but are not limited to total RNA, mRNA, and polypeptides, and derivatives of these molecules such as cDNAs and ESTs. As such, a biological sample can comprise a cell or a group of cells. Any cell or group of cells can be used with the methods of the presently claimed subject matter, although cell-types and organs that would be predicted to show differential gene expression in subjects with autoimmune disease versus normal subjects are best suited. In one embodiment, gene expression levels are determined using PBMCs as the biological sample. In one embodiment, the biological sample comprises the constituent cell types that make up a PBMC preparation including, but not limited to T cells, B cells, monocytes, natural killer (NK) cells and natural killer T (NKT) cells. Also encompassed within the phrase “biological sample” are biomolecules that are derived from a cell or group of cells that permit gene expression levels to be determined, e.g. nucleic acids and polypeptides.

The expression level of a gene can be determined using molecular biology techniques that are well known in the art. For example, if the expression level is to be determined by analyzing RNA isolated from the biological sample, techniques for determining the expression level include, but are not limited to Northern blotting, quantitative PCR, and the use of nucleic acid arrays and microarrays.

In one embodiment, the expression level of a gene is determined by hybridizing ³³P-labeled cDNA generated from total RNA isolated from a biological sample to one or more DNA sequences representing one or more genes that has been affixed to a solid support, e.g. a membrane. When a membrane comprises nucleic acids representing many genes (including internal controls), the relative expression level of many genes can be determined. The presence of internal control sequences on the membrane also allows experiment-to-experiment variations to be detected, yielding a strategy whereby the raw expression data derived from each experiment can be normalized and compared from experiment-to-experiment.

Alternatively, gene expression can be determined by analyzing protein levels in a biological sample using antibodies. Representative antibody-based techniques include, but are not limited to immunoprecipitation, Western blotting, and the use of immunoaffinity columns.

The term “subject” as used herein refers to any vertebrate species. The methods of the presently claimed subject matter are particularly useful in the diagnosis of warm-blooded vertebrates. Thus, the presently claimed subject matter concerns mammals. More particularly contemplated is the diagnosis of mammals such as humans, as well as those mammals of importance due to being endangered (such as Siberian tigers), of economical importance (animals raised on farms for consumption by humans) and/or social importance (animals kept as pets or in zoos) to humans, for instance, carnivores other than humans (such as cats and dogs), swine (pigs, hogs, and wild boars), ruminants (such as cattle, oxen, sheep, giraffes, deer, goats, bison, and camels), and horses. Also contemplated is the diagnosis of autoimmune disease in livestock, including, but not limited to domesticated swine (pigs and hogs), ruminants, horses, poultry, and the like.

II. Isolation and Analysis of Nucleic Acids

II.A. Enrichment of Nucleic Acids

The presently claimed subject matter encompasses use of a sufficiently large biological sample to enable a comprehensive survey of low abundance nucleic acids in the sample. Thus, the sample can optionally be concentrated prior to isolation of nucleic acids. Several protocols for concentration have been developed that alternatively use slide supports (Kohsaka & Carson, 1994; Millar et al., 1995), filtration columns (Bej et al., 1991), or immunomagnetic beads (Albert et al., 1992; Chiodi et al., 1992). Such approaches can significantly increase the sensitivity of subsequent detection methods.

As one example, SEPHADEX® matrix (Sigma, St. Louis, Mo., United States of America) is a matrix of diatomaceous earth and glass suspended in a solution of chaotropic agents and has been used to bind nucleic acid material (Boom et al., 1990; Buffone et al., 1991). After the nucleic acid is bound to the solid support material, impurities and inhibitors are removed by washing and centrifugation, and the nucleic acid is then eluted into a standard buffer. Target capture also allows the target sample to be concentrated into a minimal volume, facilitating the automation and reproducibility of subsequent analyses (Lanciotti et al., 1992).

II.B. Nucleic Acid Isolation

Methods for nucleic acid isolation can comprise simultaneous isolation of total nucleic acid, or separate and/or sequential isolation of individual nucleic acid types (e.g., genomic DNA, cDNA, organelle DNA, genomic RNA, mRNA, polyA⁺ RNA, rRNA, tRNA) followed by optional combination of multiple nucleic acid types into a single sample.

When total RNA or purified mRNA is selected as a biological sample, the disclosed method enables an assessment of a level of gene expression. For example, detecting a level of gene expression in a biological sample can comprise determination of the abundance of a given mRNA species in the biological sample.

RNA isolation methods are known to one of skill in the art. See Albert et al., 1992; Busch et al., 1992; Hamel et al., 1995; Herrewegh et al., 1995; Izraeli et al., 1991; McCaustland et al., 1991; Natarajan et al., 1994; Rupp et al., 1988; Tanaka et al., 1994; Vankerckhoven et al., 1994. A representative procedure for RNA isolation from a biological sample is set forth in Example 2.

Simple and semi-automated extraction methods can also be used for nucleic acid isolation, including for example, the SPLIT SECOND™ system (Boehringer Mannheim, Indianapolis, Ind., United States of America), the TRIZOL™ reagent system (Life Technologies, Gaithersburg, Md., United States of America), and the FASTPREP™ system (Bio 101, La Jolla, Calif., United States of America). See also Paladichuk 1999.

Nucleic acids that are used for subsequent amplification and labeling can be analytically pure as determined by spectrophotometric measurements or by visual inspection following electrophoretic resolution. The nucleic acid sample can be free of contaminants such as polysaccharides, proteins, and inhibitors of enzyme reactions. When an RNA sample is intended for use as probe, it can be free of nuclease contamination. Contaminants and inhibitors can be removed or substantially reduced using resins for DNA extraction (e.g., CHELEX™ 100 from BioRad Laboratories, Hercules, Calif., United States of America) or by standard phenol extraction and ethanol precipitation. Isolated nucleic acids can optionally be fragmented by restriction enzyme digestion or shearing prior to amplification.

II.C. PCR Amplification of Nucleic Acids

The terms “template nucleic acid” and “target nucleic acid” as used herein each refers to nucleic acids isolated from a biological sample as described herein above. The terms “template nucleic acid pool”, “template pool”, “target nucleic acid pool”, and “target pool” each refers to an amplified sample of “template nucleic acid”. Thus, a target pool comprises amplicons generated by performing an amplification reaction using the template nucleic acid. In one embodiment, a target pool is amplified using a random amplification procedure as described herein. In another embodiment, a target pool is amplified using a mixture of primers specific for one or more reference genes.

The term “target-specific primer” refers to a primer that hybridizes selectively and predictably to a target sequence, for example a sequence that shows differential expression in a patient with an autoimmune disease (for example, RA) relative to a normal patient, in a target nucleic acid sample. A target-specific primer can be selected or synthesized to be complementary to known nucleotide sequences of target nucleic acids.

The term “random primer”, refers to a primer having an arbitrary sequence. The nucleotide sequence of a random primer can be known, although such sequence is considered arbitrary in that it is not designed for complementarity to a nucleotide sequence of the target-specific probe. The term “random primer” encompasses selection of an arbitrary sequence having increased probability to be efficiently utilized in an amplification reaction. For example, the Random Oligonucleotide Construction Kit (ROCK; available from http://www.sru.edu/depts/artsci/bio/ROCK.htm) is a macro-based program that facilitates the generation and analysis of random oligonucleotide primers (Strain & Chmielewski, 2001). Representative primers include, but are not limited to random hexamers and rapid amplification of polymorphic DNA (RAPD)-type primers as described in Williams et al., 1990.

A random primer can also be degenerate or partially degenerate as described in Telenius et al., 1992. Briefly, degeneracy can be introduced by selection of alternate oligonucleotide sequences that can encode a same amino acid sequence.

In one embodiment, random primers can be prepared by shearing or digesting a portion of the template nucleic acid sample., Random primers so-constructed comprise a sample-specific set of random primers.

The term “heterologous primer” refers to a primer complementary to a sequence that has been introduced into the template nucleic acid pool. For example, a primer that is complementary to a linker or adaptor is a heterologous primer. Representative heterologous primers can optionally include a poly(dT) primer, a poly(T) primer, or as appropriate, a poly(dA) primer or a poly(A) primer.

The term “primer” as used herein refers to a contiguous sequence comprising in one embodiment about 6 or more nucleotides, in another embodiment about 10-20 nucleotides (e.g. 15-mer), and in still another embodiment about 20-30 nucleotides (e.g. a 22-mer). Primers provided and employed as disclosed herein encompass oligonucleotides of sufficient length and appropriate sequence so as to provide initiation of polymerization on a nucleic acid molecule.

II.C.1. Quantitative RT-PCR

In one embodiment of the presently claimed subject matter, the abundance of specific mRNA species present in a biological sample (for example, mRNA extracted from PBMCs) is assessed by quantitative RT-PCR. In this embodiment, standard molecular biological techniques are used in conjunction with specific PCR primers to quantitatively amplify those mRNA molecules corresponding to the genes of interest. Methods for designing specific PCR primers and for performing quantitative amplification of nucleic acids including mRNA are well known in the art. See e.g. Sambrook & Russell, 2001; Vandesompele et al., 2002; Joyce 2002.

II.C.2. Amplified Antisense RNA (aaRNA)

Several procedures have been developed specifically for random amplification of RNA, including but not limited to Amplified Antisense RNA (aaRNA) and Global RNA Amplification, also described further herein below. A population of RNA can be amplified using a technique referred to as Amplified Antisense RNA (aaRNA). See Van Gelder et al., 1990; Wang et al., 2000. Briefly, an oligo(dT) primer is synthesized such that the 5′ end of the primer includes a T7 RNA polymerase promoter. This oligonucleotide can be used to prime the poly(A)⁺ mRNA population to generate cDNA. Following first strand cDNA synthesis, second strand cDNA is generated using RNA nicking and priming (Sambrook & Russell, 2001). The resulting cDNA is treated briefly with S1 nuclease and blunt-ended with T4 DNA polymerase. The cDNA is then used as a template for transcription-based amplification using the T7 RNA polymerase promoter to direct RNA synthesis.

Eberwine et al. adapted the aaRNA procedure for in situ random amplification of RNA followed by target-specific amplification. The successful amplification of under represented transcripts suggests that the pool of transcripts amplified by aaRNA is representative of the initial mRNA population (Eberwine et al., 1992).

II.C.3. Global RNA Amplification

U.S. Pat. No. 6,066,457 to Hampson et al. describes a method for substantially uniform amplification of a collection of single stranded nucleic acid molecules such as RNA. Briefly, the nucleic acid starting material is anchored and processed to produce a mixture of directional shorter random size DNA molecules suitable for amplification of the sample.

In accordance with the methods of the presently claimed subject matter, any one of the above-mentioned PCR techniques or related techniques can be employed to perform the step of amplifying the nucleic acid sample. In addition, such methods can be optimized for amplification of a particular subset of nucleic acid (e.g., specific mRNA molecules versus total mRNA), and representative optimization criteria and related guidance can be found in the art. See Cha & Thilly, 1993; Linz et al., 1990; Robertson & Walsh-Weller, 1998; Roux 1995; Williams 1989; McPherson et al., 1995.

II.C.4. Kits for Gene Expression Analysis

The presently claimed subject matter also provides for kits comprising a plurality of oligonucleotide primers that can be used to assess gene expression levels of genes of interest. In non-limiting embodiments, the kit can comprise oligonucleotide primers designed to be used to determine the expression level of one or more (e.g. 1, 5, 10, 20, 30, or all) of the genes set forth in SEQ ID NOs: 1-94. Additionally, the kit can comprise instructions for using the primers including, but not limited to information regarding proper reaction conditions and the sizes of the expected amplified fragments.

III. Nucleic Acid Labeling

In one embodiment, the expression level of a gene in a biological sample is determined by hybridizing total RNA isolated from the biological sample to an array containing known quantities of nucleic acid sequences corresponding to reference genes. For example, the array can comprise single-stranded nucleic acids (also referred to herein as “probes” and/or “probe sets”) in known amounts for specific genes, which can then be hybridized to nucleic acids isolated from the biological sample. The array can be set up such that the nucleic acids are present on a solid support in such a manner as to allow the identification of those genes on the array to which the total RNA hybridizes. In this embodiment, the total RNA is hybridized to the array, and the genes to which the total RNA hybridizes are detected using standard techniques. In one embodiment of the presently claimed subject matter, the amplified nucleic acids are labeled with a radioactive nucleotide prior to hybridization to the array, and the genes on the array to which the RNA hybridizes are detected by autoradiography or phosphorimage analysis.

Alternatively, nucleic acids isolated from a biological sample are hybridized with a set of probes without prior labeling of the nucleic acids. For example, unlabeled total RNA isolated from the biological sample can be detected by hybridization to one or more labeled probes, the labeled probes being specific for those genes found to be useful in the methods of the presently claimed subject matter (e.g. those genes represented by SEQ ID NOs: 1-94). In another embodiment, both the nucleic acids and the one or more probes include a label, wherein the proximity of the labels following hybridization enables detection. An exemplary procedure using nucleic acids labeled with chromophores and fluorophores to generate detectable photonic structures is described in U.S. Pat. No. 6,162,603.

The nucleic acids or probes/probe sets can be labeled using any detectable label. It will be understood to one of skill in the art that any suitable method for labeling can be used, and no particular detectable label or technique for labeling should be construed as a limitation of the disclosed methods.

Direct labeling techniques include incorporation of radioisotopic (e.g. ³²P, ³³P, or ³⁵S) or fluorescent nucleotide analogues into nucleic acids by enzymatic synthesis in the presence of labeled nucleotides or labeled. PCR primers. A radio-isotopic label can be detected using autoradiography or phosphorimaging. A fluorescent label can be detected directly using emission and absorbance spectra that are appropriate for the particular label used. Any detectable fluorescent dye can be used, including but not limited to fluorescein isothiocyanate (FITC), FLUOR X™, ALEXA FLUOR® 488, OREGON GREEN® 488, 6-JOE (6-carboxy-4′,5′-dichloro-2′,7′-dimethoxyfluorescein, succinimidyl ester), ALEXA FLUOR® 532, Cy3, ALEXA FLUOR® 546, TMR (tetramethylrhodamine), ALEXA FLUOR® 568, ROX (X-rhodamine), ALEXA FLUOR® 594, TEXAS RED®, BODIPY® 630/650, and Cy5 (available from Amersham Pharmacia Biotech, Piscataway, N.J., United States of America, or from Molecular Probes Inc., Eugene, Oreg., United States of America). Fluorescent tags also include sulfonated cyanine dyes (available from Li-Cor, Inc., Lincoln, Nebr., United States of America) that can be detected using infrared imaging. Methods for direct labeling of a heterogeneous nucleic acid sample are known in the art and representative protocols can be found in, for example, DeRisi et al., 1996; Sapolsky & Lipshutz, 1996; Schena et al., 1995; Schena et al., 1996; Shalon et al., 1996; Shoemaker et al., 1996; Wang et al., 1998. A representative procedure is set forth herein as Example 6.

Indirect labeling techniques can also be used in accordance with the methods of the presently claimed subject matter, and in some cases, can facilitate detection of rare target sequences by amplifying the label during the detection step. Indirect labeling involves incorporation of epitopes, including recognition sites for restriction endonucleases, into amplified nucleic acids prior to hybridization with a set of probes. Following hybridization, a protein that binds the epitope is used to detect the epitope tag.

In one embodiment, a biotinylated nucleotide can be included in the amplification reactions to produce a biotin-labeled nucleic acid sample. Following hybridization of the biotin-labeled sample with probes as described herein, the label can be detected by binding of an avidin-conjugated fluorophore, for example streptavidin-phycoerythrin, to the biotin label. Alternatively, the label can be detected by binding of an avidin-horseradish peroxidase (HRP) streptavidin conjugate, followed by calorimetric detection of an HRP enzymatic product.

The quality of probe or nucleic acid sample labeling can be approximated by determining the specific activity of label incorporation. For example, in the case of a fluorescent label, the specific activity of incorporation can be determined by the absorbance at 260 nm and 550 nm (for Cy3) or 650 nm (for Cy5) using published extinction coefficients (Randolph & Waggoner, 1995). Very high label incorporation (specific activities of >1 fluorescent molecule/20 nucleotides) can result in a decreased hybridization signal compared with probe with lower label incorporation. Very low specific activity (<1 fluorescent molecule/100 nucleotides) can give unacceptably low hybridization signals. See Worley et al., 2000. Thus, it will be understood to one of skill in the art that labeling methods can be optimized for performance in various hybridization assays, and that optimal labeling can be unique to each label type.

IV. Microarrays

In one embodiment of the presently claimed subject matter, nucleic acids isolated from a biological sample are hybridized to a microarray, wherein the microarray comprises nucleic acids corresponding to those genes to be tested as well as internal control genes. The genes are immobilized on a solid support, such that each position on the support identifies a particular gene. Solid supports include, but are not limited to nitrocellulose and nylon membranes. Solid supports can also be glass or silicon-based (i.e. gene “chips”). Any solid support can be used in the methods of the presently claimed subject matter, so long as the support provides a substrate for the localization of a known amount of a nucleic acid in a specific position that can be identified subsequent to the hybridization and detection steps. In one embodiment, a microarray comprises a nylon membrane (for example, the GF211 Human “Named Genes” GENEFILTERS® Microarrays Release 1 available from RESGEN™).

A microarray can be assembled using any suitable method known to one of skill in the art, and any one microarray configuration or method of construction is not considered to be a limitation of the disclosure. Representative microarray formats that can be used are described herein below.

IV.A. Array Substrate and Configuration

The substrate for printing the array should be substantially rigid and amenable to DNA immobilization and detection methods (e.g., in the case of fluorescent detection, the substrate must have low background fluorescence in the region of the fluorescent dye excitation wavelengths). The substrate can be nonporous or porous as determined most suitable for a particular application. Representative substrates include, but are not limited to a glass microscope slide, a glass coverslip, silicon, plastic, a polymer matrix, an agar gel, a polyacrylamide gel, and a membrane, such as a nylon, nitrocellulose or ANAPORE™ (Whatman, Maidstone, United Kingdom) membrane.

Porous substrates (membranes and polymer matrices) are preferred in that they permit immobilization of relatively large amount of probe molecules and provide a three-dimensional hydrophilic environment for biomolecular interactions to occur (Dubiley et al., 1997; Yershov et al., 1996). A BIOCHIP ARRAYER™ dispenser (Packard Instrument Company, Meriden, Conn., United States of America) can effectively dispense probes onto membranes such that the spot size is consistent among spots whether one, two, or four droplets were dispensed per spot (Englert 2000). The array can also comprise a dot blot or a slot blot.

A microarray substrate for use in accordance with the methods of the presently claimed subject matter can have either a two-dimensional (planar) or a three-dimensional (non-planar) configuration. An exemplary three-dimensional microarray is the FLOW-THRU™ chip (Gene Logic, Inc., Gaithersburg, Md., United States of America), which has implemented a gel pad to create a third dimension. Such a three-dimensional microarray can be constructed of any suitable substrate, including glass capillary, silicon, metal oxide filters, or porous polymers. See Yang et al., 1998; Steel et al., 2000.

Briefly, a FLOW-THRU™ chip (Gene Logic, Inc.) comprises a uniformly porous substrate having pores or microchannels connecting upper and lower faces of the chip. Probes are immobilized on the walls of the microchannels and a hybridization solution comprising sample nucleic acids can flow through the microchannels. This configuration increases the capacity for probe and target binding by providing additional surface relative to two-dimensional arrays. See U.S. Pat. No. 5,843,767.

IV.B. Surface Chemistry

The particular surface chemistry employed is inherent in the microarray substrate and substrater preparation. Immobilization of nucleic acids probes post-synthesis can be accomplished by various approaches, including adsorption, entrapment, and covalent attachment. Preferably, the binding technique does not disrupt the activity of the probe.

For substantially permanent immobilization, covalent attachment is preferred. Since few organic functional groups react with an activated silica surface, an intermediate layer is advisable for substantially permanent probe immobilization. Functionalized organosilanes can be used as such an intermediate layer on glass and silicon substrates (Liu & Hlady, 1996; Shriver-Lake 1998). A hetero-bifunctional cross-linker requires that the probe have a different chemistry than the surface, and is preferred to avoid linking reactive groups of the same type. A representative hetero-bifunctional cross-linker comprises gamma-maleimidobutyryloxy-succimide (GMBS) that can bind maleimide to a primary amine of a probe. Procedures for using such linkers are known to one of skill in the art and are summarized in Hermanson 1990. A representative protocol for covalent attachment of DNA to silicon wafers is described in O'Donnell et al., 1997.

When using a glass substrate, the glass should be substantially free of debris and other deposits and have a substantially uniform coating. Pretreatment of slides to remove organic compounds that can be deposited during their manufacture can be accomplished, for example, by washing in hot nitric acid. Cleaned slides can then be coated with 3-aminopropyltrimethoxysilane using vapor-phase techniques. After silane deposition, slides are washed with deionized water to remove any silane that is not attached to the glass and to catalyze unreacted methoxy groups to cross-link to neighboring silane moieties on the slide. The uniformity of the coating can be assessed by known methods, for example electron spectroscopy for chemical analysis (ESCA) or ellipsometry (Ratner & Castner, 1997; Schena et al., 1995). See also Worley et al., 2000.

For attachment of probes greater than about 300 base pairs, noncovalent binding is suitable. A representative technique for noncovalent linkage involves use of sodium isothiocyanate (NaSCN) in the spotting solution, as described in Example 8. When using this method, amino-silanized slides can be used since this coating improves nucleic acid binding when compared to bare glass. This method works well for spotting applications that use about 100 ng/μl (Worley et al., 2000).

In the case of nitrocellulose or nylon membranes, the chemistry of nucleic acid binding to these membranes has been well characterized (Southern 1975; Sambrook & Russell, 2001). One such nylon filter array is the GF211 Human “Named Genes” GENEFILTERS® Microarrays Release 1 (available from RESGEN™, a division of Invitrogen Corporation, Calsbad, Calif., United States of America), although other arrays can also be used.

IV.C. Arraying Techniques

A microarray for the detection of gene expression levels in a biological sample can be constructed using any one of several methods available in the art including, but not limited to photolithographic and microfluidic methods, further described herein below. In one embodiment, the method of construction is flexible, such that a microarray can be tailored for a particular purpose.

As is standard in the art, a technique for making a microarray should create consistent and reproducible spots. Each spot can be uniform, and appropriately spaced away from other spots within the configuration. A solid support for use in the presently claimed subject matter comprises in one embodiment about 10 or more spots, in another embodiment about 100 or more spots, in another embodiment about 1,000 or more spots, and in still another embodiment about 10,000 or more spots. In one embodiment, the volume deposited per spot is about 10 picoliters to about 10 nanoliters, and in another embodiment about 50 picoliters to about 500 picoliters. The diameter of a spot is in one embodiment about 50 μm to about 1000 μm, and in another embodiment about 100 μm to about 250 μm.

Light-directed synthesis. This technique was developed by Fodor et al. (Fodor et al., 1991; Fodor et al., 1993; U.S. Pat. No. 5,445,934), and commercialized by Affymetrix, Inc. of Santa Clara, Calif., United States of America. Briefly, the technique uses precision photolithographic masks to define the positions at which single, specific nucleotides are added to growing single-stranded nucleic acid chains. Through a stepwise series of defined nucleotide additions and light-directed chemical linking steps, high-density arrays of defined oligonucleotides are synthesized on a solid substrate. A variation of the method, called Digital Optical Chemistry, employs mirrors to direct light synthesis in place of photolithographic masks (International Publication No. WO 99/63385). This approach is generally limited to probes of about 25 nucleotides in length or less. See also Warrington et al., 2000.

Contact Printing. Several procedures and tools have been developed for printing microarrays using rigid pin tools. In surface contact printing, the pin tools are dipped into a sample solution, resulting in the transfer of a small volume of fluid onto the tip of the pins. Touching the pins or pin samples onto a microarray surface leaves a spot, the diameter of which is determined by the surface energies of the pin, fluid, and microarray surface. Typically, the transferred fluid comprises a volume in the nanoliter or picoliter range.

One common contact printing technique uses a solid pin replicator. A replicator pin is a tool for picking up a sample from one stationary location and transporting it to a defined location on a solid support. A typical configuration for a replicating head is an array of solid pins, generally in an 8×12 format, spaced at 9-mm centers that are compatible with 96- and 384-well plates. The pins are dipped into the wells, lifted, moved to a position over the microarray substrate, lowered to touch the solid support, whereby the sample is transferred. The process is repeated to complete transfer of all the samples. See Maier et al., 1994. A recent modification of solid pins involves the use of solid pin tips having concave bottoms, which print more efficiently than flat pins in some circumstances. See Rose 2000.

Solid pins for microarray printing can be purchased, for example, from TeleChem International, Inc. of Sunnyvale, Calif. in a wide range of tip dimensions. The CHIPMAKER™ and STEALTH™ pins from TeleChem contain a stainless steel shaft with a fine point. A narrow gap is machined into the point to serve as a reservoir for sample loading and spotting. The pins have a loading volume of 0.2 μl to 0.6 μl to create spot sizes ranging from 75 μm to 360 μm in diameter.

To permit the printing of multiple arrays with a single sample loading, quill-based et al. tools, including printing capillaries, tweezers, and split pins have been developed. These printing tools hold larger sample volumes than solid pins and therefore allow the printing of multiple arrays following a single sample loading. Quill-based arrayers withdraw a small volume of fluid into a depositing device from a microwell plate by capillary action. See Schena et al., 1995. The diameter of the capillary typically ranges from about 10 μm to about 100 μm. A robot then moves the head with quills to the desired location for dispensing. The quill carries the sample to all spotting locations, where a fraction of the sample is deposited. The forces acting on the fluid held in the quill must be overcome for the fluid to be released. Accelerating and then decelerating by impacting the quill on a microarray substrate accomplishes fluid release. When the tip of the quill hits the solid support, the meniscus is extended beyond the tip and transferred onto the substrate. Carrying a large volume of sample fluid minimizes spotting variability between arrays. Because tapping on the surface is required for fluid transfer, a relatively rigid support, for example a glass slide, is appropriate for this method of sample delivery.

A variation of the pin printing process is the PIN-AND-RING™ technique developed by Genetic MicroSystems Inc. of Woburn, Mass., United States of America. This technique involves dipping a small ring into the sample well and removing it to capture liquid in the ring. A solid pin is then pushed through the sample in the ring, and the sample trapped on the flat end of the pin is deposited onto the surface. See Mace et al., 2000. The PIN-AND-RING™ technique is suitable for spotting onto rigid supports or soft substrates such as agar, gels, nitrocellulose, and nylon. A representative instrument that employs the PIN-AND-RING™ technique is the 417™ Arrayer available from Affymetrix, Inc. of Santa Clara, Calif., United States of America.

Additional procedural considerations relevant to contact printing methods, including array layout options, print area, print head configurations, sample loading, preprinting, microarray surface properties, sample solution properties, pin velocity, pin washing, printing time, reproducibility, and printing throughput are known in the art, and are summarized in Rose 2000.

Noncontact Ink-Jet Printing. A representative method for noncontact ink-jet printing uses a piezoelectric crystal closely apposed to the fluid reservoir. One configuration places the piezoelectric crystal in contact with a glass capillary that holds the sample fluid. The sample is drawn up into the reservoir and the crystal is biased with a voltage, which causes the crystal to deform, squeeze the capillary, and eject a small amount of fluid from the tip. Piezoelectric pumps offer the capability of controllable, fast jetting rates and consistent volume deposition. Most piezoelectric pumps are unidirectional pumps that need to be directly connected, for example by flexible capillary tubing, to a source of sample supply or wash solution. The capillary and jet orifices should be of sufficient inner diameter so that molecules are not sheared. The void volume of fluid contained in the capillary typically ranges from about 100 μl to about 500 μl and generally is not recoverable. See U.S. Pat. No. 5,965,352.

Devices that provide thermal pressure, sonic pressure, or oscillatory pressure on a liquid stream or surface can also be used for ink-jet printing. See Theriault et al., 1999.

Syringe-Solenoid Printing. Syringe-solenoid technology combines a syringe pump with a microsolenoid valve to provide quantitative dispensing of nanoliter sample volumes. A high-resolution syringe pump is connected to both a high-speed microsolenoid valve and a reservoir through a switching valve. For printing microarrays, the system is filled with a system fluid, typically water, and the syringe is connected to the microsolenoid valve. Withdrawing the syringe causes the sample to move upward into the tip. The syringe then pressurizes the system such that opening the microsolenoid valve causes droplets to be ejected onto the surface. With this configuration, a minimum dispense volume is on the order of 4 nl to 8 nl. The positive displacement nature of the dispensing mechanism creates a substantially reliable system. See U.S. Pat. Nos. 5,743,960 and 5,916,524.

Electronic Addressing. This method involves placing charged molecules at specific positions on a blank microarray substrate, for example a NANOCHIP™ substrate (Nanogen Inc., San Diego, Calif., United States of America). A nucleic acid probe is introduced to the microchip, and the negatively-charged probe moves to the selected charged position, where it is concentrated and bound. Serial application of different probes can be performed to assemble an array of probes at distinct positions. See U.S. Pat. No. 6,225,059 and International Publication No. WO 01/23082.

Nanoelectrode Synthesis. An alternative array that can also be used in accordance with the methods of the presently claimed subject matter provides ultra small structures (nanostructures) of a single or a few atomic layers synthesized on a semiconductor surface such as silicon. The nanostructures can be designed to correspond precisely to the three-dimensional shape and electro-chemical properties of molecules, and thus can be used to recognize nucleic acids of a particular nucleotide sequence. See U.S. Pat. No. 6,123,819.

V. Hybridization

V.A. General Considerations

As mentioned above, the terms “specifically hybridizes” and selectively hybridizes each refer to binding, duplexing, or hybridizing of a molecule only to a particular nucleotide sequence under stringent conditions when that sequence is present in a complex nucleic acid mixture (e.g., total cellular DNA or RNA).

As mentioned above, the phrase “substantially hybridizes” refers to complementary hybridization between a probe nucleic acid molecule and a substantially identical target nucleic acid molecule as defined herein. Substantial hybridization is generally permitted by reducing the stringency of the hybridization conditions using art-recognized techniques.

“Stringent hybridization conditions” and “stringent hybridization wash conditions” in the context of nucleic acid hybridization experiments are both sequence- and environment-dependent. Longer sequences hybridize specifically at higher temperatures. Generally, highly stringent hybridization and wash conditions are selected to be about 5° C. lower than the thermal melting point (Tₘ) for the specific sequence at a defined ionic strength and pH. The Tₘ is the temperature (under defined ionic strength and pH) at which 50% of the target sequence hybridizes to a perfectly matched probe. Very stringent conditions are selected to be equal to the Tₘ for a particular probe. Typically, under “stringent conditions” a probe hybridizes specifically to its target sequence, but to no other sequences.

An extensive guide to the hybridization of nucleic acids is found in Tijssen 1993. In general, a signal to noise ratio of 2-fold (or higher) than that observed for a negative control probe in a same hybridization assay indicates detection of specific or substantial hybridization.

It is understood that in order to determine a gene expression level by hybridization, a full-length cDNA need not be employed. To determine the expression level of a gene represented by one of SEQ ID NOs: 1-94, any representative fragment or subsequence of the sequences set forth in SEQ ID NOs: 1-94 can be employed in conjunction with the hybridization conditions disclosed hereinabove. As a result, a nucleic acid sequence used to assay a gene expression level can comprise sequences corresponding to the open reading frame (or a portion thereof), the 5′ untranslated region, and/or the 3′ untranslated region. It is understood that any sequence that will allow the expression level of a particular gene to be specifically determined can be used.

V.B. Hybridization on a Solid Support

In another embodiment of the presently claimed subject matter, an amplified and labeled nucleic acid sample is hybridized to probes or probe sets that are immobilized on a continuous solid support comprising a plurality of identifying positions.

Representative hybridization conditions are set forth herein. For some high-density glass-based microarray experiments, hybridization at 65° C. is too stringent for typical use, at least in part because the presence of fluorescent labels destabilizes the nucleic acid duplexes (Randolph & Waggoner, 1997). Alternatively, hybridization can be performed in a formamide-based hybridization buffer as described in Piétu et al., 1996.

A microarray format can be selected for use based on its suitability for electrochemical-enhanced hybridization. Provision of an electric current to the microarray, or to one or more discrete positions on the microarray facilitates localization of a target nucleic acid sample near probes immobilized on the microarray surface. Concentration of target nucleic acid near arrayed probe accelerates hybridization of a nucleic acid of the sample to a probe. Further, electronic stringency control allows the removal of unbound and nonspecifically bound DNA after hybridization. See U.S. Pat. Nos. 6,017,696 and 6,245,508.

V.C. Hybridization in Solution

In another embodiment of the presently claimed subject matter, an amplified and labeled nucleic acid sample is hybridized to one or more probes in solution. Representative stringent hybridization conditions for complementary nucleic acids having more than about 100 complementary residues are overnight hybridization in 50% formamide with 1 mg of heparin at 42° C. An example of highly stringent wash conditions is 15 minutes in 0.1×SSC, 5M NaCl at 65° C. An example of stringent wash conditions is 15 minutes in 0.2×SSC buffer at 65° C. (See Sambrook & Russell, 2001 for a description of SSC buffer). A high stringency wash can be preceded by a low stringency wash to remove background probe signal. An example of medium stringency wash conditions for a duplex of more than about 100 nucleotides, is 15 minutes in 1×SSC at 45° C. An example of low stringency wash for a duplex of more than about 100 nucleotides, is 15 minutes in 4-6×SSC at 40° C. Stringent conditions can also be achieved with the addition of destabilizing agents such as formamide.

For short probes (e.g., about 10 to 50 nucleotides), stringent conditions typically involve salt concentrations of less than about 1 M Na⁺ ion, typically about 0.01 M to 1 M Na⁺ ion concentration (or other salts) at pH 7.0-8.3, and the temperature is typically at least about 30° C.

Optionally, nucleic acid duplexes or hybrids can be captured from the solution for subsequent analysis, including detection assays. For example, in a simple assay, a single probe set is hybridized to an amplified and labeled RNA sample derived from a target nucleic acid sample. Following hybridization, an antibody that recognizes DNA:RNA hybrids is used to precipitate the hybrids for subsequent analysis. The expression level of the gene is determined by detection of the label in the precipitate.

Alternate capture techniques can be used as will be understood to one of skill in the art, for example, purification by a metal affinity column when using probes comprising a histidine tag. As another example, the hybridized sample can be hydrolyzed by alkaline treatment wherein the double-stranded hybrids are protected while non-hybridizing single-stranded template and excess probe are hydrolyzed. The hybrids are then collected using any nucleic acid purification technique for further analysis.

To determine the expression levels of multiple genes simultaneously, probes or probe sets can be distinguished by differential labeling of probes or probe sets. Alternatively, probes or probe sets can be spatially separated in different hybridization vessels. Representative embodiments of each approach are described herein below.

In one embodiment, a probe or probe set having a unique label is prepared for each gene to be analyzed. For example, a first probe or probe set can be labeled with a first fluorescent label, and a second probe or probe set can be labeled with a second fluorescent label. Multi-labeling experiments should consider label characteristics and detection techniques to optimize detection of each label. Representative first and second fluorescent labels are Cy3 and Cy5 (Amersham Pharmacia Biotech, Piscataway, N.J., United States of America), which can be analyzed with good contrast and minimal signal leakage.

A unique label for each probe or probe set can further comprise a labeled microsphere to which a probe or probe set is attached. A representative system is LabMAP (Luminex Corporation, Austin, Tex., United States of America). Briefly, LabMAP (Laboratory Multiple Analyte Profiling) technology involves performing molecular reactions, including hybridization reactions, on the surface of color-coded microscopic beads called microspheres. When used in accordance with the methods disclosed herein, an individual probe or probe set is attached to beads having a single color-code such that they can be identified throughout the assay. Successful hybridization is measured using a detectable label of the amplified nucleic acid sample, wherein the detectable label can be distinguished from each color-code used to identify individual microspheres. Following hybridization of the amplified, labeled nucleic acid sample with a set of microspheres comprising probe sets, the hybridization mixture is analyzed to detect the signal of the color-code as well as the label of a sample nucleic acid bound to the microsphere. See Vignali 2000; Smith et al., 1998; International Publication Nos. WO 01/13120, WO 01/14589, WO 99/19515, and WO 97/14028.

VI. Detection

Methods for detecting a hybridization duplex or triplex are selected according to the label employed.

In the case of a radioactive label (e.g., ³²P-, ³³P-, or ³⁵S-dNTP) detection can be accomplished by autoradiography or by using a phosphorimager as is known to one of skill in the art. In one embodiment, a detection method can be automated and is adapted for simultaneous detection of numerous samples.

Common research equipment has been developed to perform high-throughput fluorescence detecting, including instruments from GSI Lumonics (Watertown, Mass., United States of America), Amersham Pharmacia Biotech/Molecular Dynamics (Sunnyvale, Calif., United States of America), Applied Precision Inc. (Issauah, Wash., United States of America), Genomic Solutions Inc. (Ann Arbor, Mich., United States of America), Genetic MicroSystems Inc. (Woburn, Mass., United States of America), Axon (Foster City, Calif., United States of America), Hewlett Packard (Palo Alto, Calif., United States of America), and Virtek (Woburn, Mass., United States of America). Most of the commercial systems use some form of scanning technology with photomultiplier tube detection. Criteria for consideration when analyzing fluorescent samples are summarized by Alexay et al., 1996.

In another embodiment, a nucleic acid sample or probes are labeled with far infrared, near infrared, or infrared fluorescent dyes. Following hybridization, the mixture of amplified nucleic acids and probes is scanned photoelectrically with a laser diode and a sensor, wherein the laser scans with scanning light at a wavelength within the absorbance spectrum of the fluorescent label, and light is sensed at the emission wavelength of the label. See U.S. Pat. Nos. 6,086,737; 5,571,388; 5,346,603; 5,534,125; 5,360,523; 5,230,781; 5,207,880; and 4,729,947. An ODYSSEY™ infrared imaging system (Li-Cor, Inc., Lincoln, Nebr., United States of America) can be used for data collection and analysis.

If an epitope label has been used, a protein or compound that binds the epitope can be used to detect the epitope. For example, an enzyme-linked protein can be subsequently detected by development of a colorimetric or luminescent reaction product that is measurable using a spectrophotometer or luminometer, respectively.

In one embodiment, INVADER® technology (Third Wave Technologies, Madison, Wis., United States of America) is used to detect target nucleic acid/probe complexes. Briefly, a nucleic acid cleavage site (such as that recognized by a variety of enzymes having 5′ nuclease activity) is created on a target sequence, and the target sequence is cleaved in a site-specific manner, thereby indicating the presence of specific nucleic acid sequences or specific variations thereof. See U.S. Pat. Nos. 5,846,717; 5,985,557; 5,994,069; 6,001,567; and 6,090,543.

In another embodiment, target nucleic acid/probe complexes are detected using an amplifying molecule, for example a poly-dA oligonucleotide as described in Lisle et al., 2001. Briefly, a tethered probe is employed against a target nucleic acid having a complementary nucleotide sequence. A target nucleic acid having a poly-dT sequence, which can be added to any nucleic acid sequence using methods known to one of skill in the art, hybridizes with an amplifying molecule comprising a poly-dA oligonucleotide. Short oligo-dT₄₀ signaling moieties are labeled with any suitable label (e.g., fluorescent, chemiluminescent, radioisotopic labels). The short oligo-dT₄₀ signaling moieties are subsequently hybridized along the molecule, and the label is detected.

Surface plasmon resonance spectroscopy can also be used to detect hybridization duplexes formed between a randomly amplified nucleic acid and a probe as disclosed herein. See e.g., Heaton et al., 2001; Nelson et al., 2001; Guedon et al., 2000.

VII. Rheumatoid Arthritis Gene Expression Equations

VII.A. General Description of the Equations

Genes showing differential expression between early and established RA patients were examined to determine whether expression levels could be used to classify the RA patients. The general approach for classifying subjects based upon gene expression is described in Maas et al., 2002. As disclosed herein, for each reference gene, an expression level was determined in each subject. For each reference gene, the average expression level in the early RA group was added to the average expression level in the established RA group, and the sum divided by two to arrive at an average expression level in all RA subjects. Each subject was then scored by assigning a value of 1 for each gene that in that subject was expressed at a level that was above the average expression level of that gene in all RA subjects determined above, and by assigning a value of 0 for each gene that in that subject was expressed at a level that was below the average expression level in all RA subjects. Two equations were generated that were capable of distinguishing between early and established RA patients.

The first, Equation 1, used the 10 genes that are listed in Table 1. These genes were upregulated by at least 4-fold in established RA compared to early RA. For Equation 1, the maximum score a subject could receive is 10, and the minimum score is 0. The second equation, Equation 2, used the 8 genes that are listed in Table 2. Thus, the maximum score for Equation 2 is 8, and the minimum score is 0. These genes were upregulated by at least 3-fold in the early RA patients.

TABLE 1
Genes Used in Equation 1

| Gene | Description |
|---|---|
| B2M | β₂-microglobulin (SEQ ID NOs: 33 and 34) |
| HLA-DRA | MHC, class II, DR α (SEQ ID NOs: 37 |
| | and 38) |
| SAT | Spermidine/spermine N1- |
| | acetyltransferase (SEQ ID NOs: 39 |
| | and 40) |
| SSX3 | Synovial sarcoma, X breakpoint 3 |
| | (SEQ ID NOs: 43 and 44) |
| SAS | Sarcoma amplified sequence (SEQ ID |
| | NOs: 51 and 52) |
| CHI3L1 | Chitinase 3-like 1; cartilage |
| | glycoprotein-39 (SEQ ID NOs: 69 |
| | and 70) |
| RGS4 | Regulator of G-protein signaling 4 |
| | (SEQ ID NOs: 73 and 74) |
| HBZ | Hemoglobin zeta (SEQ ID NOs: 75 |
| | and 76) |
| EEF2 | Eukaryotic translation elongation |
| | factor 2 (SEQ ID NOs: 77 and 78) |
| CHES1 | Checkpoint suppressor 1 (SEQ ID |
| | NOs: 93 and 94 |

TABLE 2
Genes Used in Equation 2

| Gene | Description |
|---|---|
| CSF3R | Colony stimulating factor 3 receptor, |
| | granulocyte (SEQ ID NOs: 3 and 4) |
| TGFBR2 | TGF-β receptor II, 70-80 kD (SEQ ID |
| | NOs: 5 and 6) |
| CYP3A4 | Cytochrome P450, subfamily IIIA; |
| | niphedipine oxidase, polypeptide 4 |
| | (SEQ ID NOs: 7 and 8) |
| HSD11B2 | hydroxysteroid (11-β) dehydrogenase |
| | 2 (SEQ ID NOs: 9 and 10) |
| TNNI2 | Troponin I, skeletal, fast (SEQ ID |
| | NOs: 11 and 12) |
| SNTA1 | Syntrophin α1; dystrophin-associated |
| | protein A1, 59 kD, acidic component |
| | (SEQ ID NOs: 13 and 14) |
| TNNT2 | Troponin T2, cardiac (SEQ ID NOs: |
| | 15 and 16) |
| ZNF74 | Zinc finger protein 74; Cos52 (SEQ ID |
| | NOs: 17 and 18) |

VII.B. Use of the Equations to Predict a Predisposition to Developing Established RA

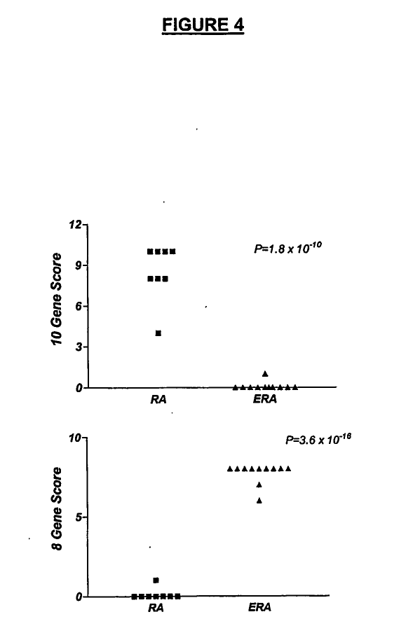

As shown in FIG. 4, each of Equations 1 and 2 allowed accurate classification of subjects in the two groups. For Equation 1, the mean (±standard error of the mean; SEM) for the group of established RA patients was 8.5±0.7 compared to 0.09±0.09 for the early RA patients (P=1.8×10⁻¹⁰). Equation 2 produced a mean value in the established group of 0.13±0.13 compared to a corresponding value in the early RA patients of 7.23±0.19 (P=3.6×10⁻¹⁶).

While applicants do not wish to be bound by any particular theory of operation, it is likely that during the transition from early stage RA to established RA, the expression levels of the genes identified as either upregulated or downregulated in early vs. established RA changes. As a result, it would be expected that for patients in the transition period, the scores that would be calculated for them using Equations 1 and 2 would be intermediate between those assigned to the subjects in the early and established RA populations. Thus, for example, a subject in the early stages of the transition would be expected to have a score of greater than about 1 but less than about 6 using Equation 1 and less than about 7 but greater than about 2 using Equation 2.

### EXAMPLES

The following Examples provide illustrative embodiments. Certain aspects of the following Examples are described in terms of techniques and procedures found or contemplated by the present inventors to work well in the practice of the embodiments. In light of the present disclosure and the general level of skill in the art, those of skill will appreciate that the following Examples are intended to be exemplary only and that numerous changes, modifications, and alterations can be employed without departing from the scope of the presently claimed subject matter.

### Example 1

### Patient Populations

Patients were recruited from the rheumatology clinics at Vanderbilt University (Nashville, Tenn., United States of America) and from the private rheumatology practice at Baptist Hospital, Nashville, Tenn. All patients satisfied diagnostic criteria for RA according to Arnett et al., 1988. Briefly, these criteria include morning softness around the joints; arthritis in three or more joint areas, including arthritis in the hands and wrists that is bilaterally symmetric; the presence of rheumatoid nodules; the presence of rheumatoid factor; and characteristic X-ray changes. The presence of at least four of these criteria at any time observed by a physician and present for at least six weeks is diagnostic of RA. Disease duration, medications, and demographic variables were determined from chart review and are summarized in Table 3.

TABLE 3
Clinical Features of Patients with Early or Established
Rheumatoid Arthritis

| | Early RA | Established RA | |
|---|---|---|---|
| | (N = 11) | (N = 9) | P** |
| Gender (% female) | 82 | 100 | 0.3 |
| Age (years) | 56 ± 4 | 60 ± 3 | 0.6 |
| Duration (years)* | 1 ± 0.2 | 10 ± 3 | 0.0039 |
| DMARD use (%) | 90 | 100 | 0.93 |
| Prednisone use (%) | 50 | 22 | 0.16 |
| MTX weekly dose (mg)* | 11 ± 1 | 14 ± 3 | 0.94 |

*values represent mean ± SEM
**P values calculated by Chi-square or Student's t-test

### Example 2

### Sample Preparation

Peripheral blood mononuclear cells (PBMC) were isolated from 20 ml of heparinized blood by centrifugation on Ficoll gradients (Sigma-Aldrich, St. Louis, Mo., United States of America). Leukocyte distribution in PBMC was determined by flow cytometry. Total RNA was isolated with TRI-REAGENT® (Molecular Research Center, Cincinnati, Ohio, United States of America), reverse transcribed with ³³P-dCTP, and 5 μg were hybridized to a GF211 membrane (RESGEN™, a division of Invitrogen Corporation, Carlsbad, Calif., United States of America). Filters were exposed to imaging screens for 24 hours and screens were scanned using a PHOSPHORIMAGER™ device (Molecular Dynamics, Piscataway, N.J., United States of America). Data were normalized to yield an average intensity of 1.0 for each clone (4329 clones total) represented on the microarray. Reproducibility of the method was established by performing replicate hybridizations to separate microarrays. Linear regression analysis demonstrated that separate hybridizations yielded R² values ranging from 0.87 to 0.96. Different exposure lengths of identical filters also produced high R² values (0.99).

### Example 3

### Data Analysis

Eisen's Cluster and Treeview software (Stanford University, Palo Alto, Calif., United States of America; Eisen et al., 1998) were used to compare similarities among individual samples. Data sets were analyzed using hierarchical, K-means, and self-organizing map algorithms (Sherlock, 2000). The RESGEN™ PATHWAYS™ 3.0 microarray analysis program (version 4 currently available from Invitrogen Corp., Carlsbad, Calif., United States of America) was used to identify differentially expressed genes in the immune and autoimmune disease classes. Gene expression data were filtered to eliminate any genes that showed less than 3 standard deviations (SD) variability in the clustering analysis. The remaining genes in the data set were clustered using an unsupervised K-means clustering algorithm with ten centroids (Eisen et al., 1998; Sherlock, 2000). Gene expression levels in the two RA groups were compared using an unpaired Student's t-test. P values of less than 0.05 were considered significant.

### Example 4

### Clustering with a Self-Organizing Map Algorithm on Genes Filtered for 3 SD Variability

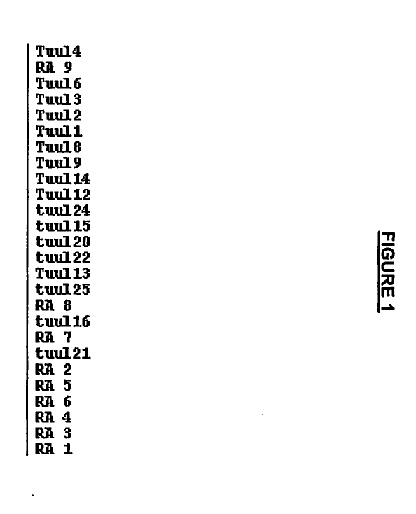

Clustering with a self-organizing map algorithm on genes filtered for 3 SD variability revealed almost complete separation of the early RA patients from the established RA patients. See FIG. 1. One patient with longstanding disease (RA8; 20 years duration) was embedded within the group of patients with early RA in this clustering analysis. Patient RA9, a subject with early disease recruited separately from the other early RA patients, clustered with these other early RA patients.

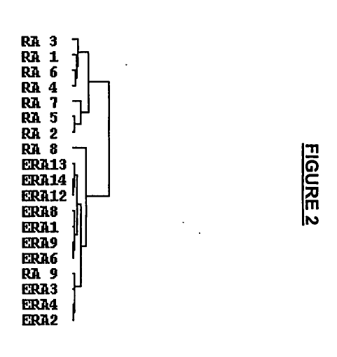

The hierarchical clustering algorithm separated the patients into two main clusters. See FIG. 2. One cluster contained 7 of the 8 established RA patients. The other cluster included all of the early RA patients, including early RA patient RA9, as well as patient RA8. RA8 was clustered somewhat separately from the early RA patients, as shown in FIG. 2.

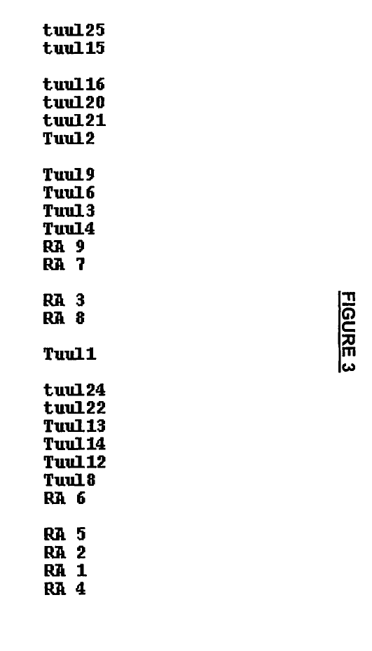

A third algorithm, K-means clustering, showed less definite separation of the two RA groups. See FIG. 3. Of the three main clusters formed by this approach, one contained four longstanding patients, and one contained five patients with early disease along with patient RA8, who had been clustered with early RA patients in the other two analyses. The third and largest cluster included both early and established RA patients; although some relatedness was suggested by the subgroups.

### Example 5

### Differential Expression of Genes in Early vs. Established RA

Gene expression values were determined as described in Example 2. The mean expression values for each gene in the early RA group and the established RA group were compared. Genes that showed greater than a 3-fold difference and high statistical significance (P<0.0005) in expression level between the two groups were identified. Nine genes were upregulated in early RA compared to established RA (Table 4). Of these, three had immune system activities: TGF-β receptor II, CSF3 receptor, and cleavage stimulation factor; and two influence levels or activity of glucocorticoids: cytochrome P450 subfamily IIIA and 11-β hydroxysteroid dehydrogenase 2. The upregulated early RA genes did not show chromosomal clustering.

TABLE 4
Genes Upregulated More Then 3-Fold in Early RA
Compared to Established RA

| | | Chromo- | |
|---|---|---|---|
| | Gene | somal | |
| Category | Designation | Location | Description |
| Immune/ | AA293218 | Xq22.1 | Cleavage stimulation factor; |
| Growth | | | increases with B cell |
| Factor | | | activation (SEQ ID NOs: 1 |
| | | | and 2) |
| | AA458507 | 1p34.3-35 | CSF 3 receptor, granulocyte |
| | | | (SEQ ID NOs: 3 and 4) |
| | AA487034 | 3p24.1 | TGF β Receptor II (SEQ ID |
| | | | NOs: 5 and 6) |
| Metabolism | R91078 | 7q22.1 | Cytochrome P450 subfamily |
| | | | 3A4 (SEQ ID NOs: 7 and 8) |
| | W95083 | 16q22 | 11β hydroxysteroid |
| | | | dehydrogenase 2 (SEQ ID |
| | | | NOs: 9 and 10) |
| Neuro- | AA181334 | 11p15.5 | Troponin I, skeletal fast twitch |
| Muscular | | | (SEQ ID NOs: 11 and 12) |
| | AA699926 | 20q11.2 | Syntropin alpha, |
| | | | neuromuscular junction |
| | | | protein (SEQ ID NOs: 13 |
| | | | and 14) |
| | N70734 | 1q32 | Troponin T2, cardiac (SEQ ID |
| | | | NOs: 15 and 16) |
| Transcription | AA629838 | 22q11.2 | Zinc finger protein 74; Cos52 |
| | | | (SEQ ID NOs: 17 and 18) |

Forty-four genes were upregulated in established RA compared to early RA, several of which could be grouped into functional categories (shown in Table 5). The largest category included 10 genes related to immune and inflammatory functions, including three MHC proteins, one related to the class I pathway (β₂-microglobulin), and two related to class II (DP α1 and DR α), as well as an interferon gamma inducible protein (IFNγ-inducible protein 30) involved in MHC restricted processing of antigen (Arunachalam et al., 2000) and nuclease sensitive element binding protein 1, a negative regulator for MHC Class II genes (Didier et al., 1988). Another gene, mannose-binding lectin-1, is related to the innate immune response; low levels are thought to be predictive of a poor prognosis in patients with early synovitis (Saevarsdottir et al., 2001).

The second category contained nine genes that were related in various ways to neoplasia or metastasis, either as tumor-associated markers or as proteins involved in the processes of proliferation, differentiation, or transformation. In addition, three genes were identified as being related to growth factors (EGF and TGF-β) that have prominent activities in both neoplasia and in the immune system. See Didier et al., 1988; Davies et al., 1999; Mendelsohn & Baselga, 2000; Leveen et al., 2002. Two genes were related to cartilage and bone: BMP4 and cartilage glycoprotein-39 (also called YKL-4). BMP4 is a member of the TGF-β superfamily. Leong & Brickell, 1996; Baeten et al., 2000. Other upregulated genes included three related to actin polymerization, two translation factors, and two Golgi proteins.

TABLE 5
Genes Upregulated More Then 3-Fold in Established RA
Compared to Early RA

| | | Chromo- | |
|---|---|---|---|
| | Gene | somal | |
| Category | Designation | Location | Description |
| Immune/ | AA036881 | 3p21.31 | Chemokine receptor, c-c motif |
| Inflam- | | | (SEQ ID NOs: 19 and 20) |
| matory | AA436163 | 9q34.11 | Prostaglandin E synthase |
| | | | (SEQ ID NOs: 21 and 22) |
| | AA446103 | 18q21.3-22 | Mannose-binding lectin 1 |
| | | | (SEQ ID NOs: 23 and 24) |
| | AA599175 | 1p34 | Nuclease sensitive element |
| | | | binding protein (SEQ ID |
| | | | NOs: 25 and 26) |
| | AA625981 | 20p13 | FK506 binding protein 1A |
| | | | (SEQ ID NOs: 27 and 28) |
| | AA630800 | 19p13.1 | IFNγ-inducible protein 30 |
| | | | (SEQ ID NOs: 29 and 30) |
| | AA634028 | 6p21.3 | MHC class II DP α1 (SEQ ID |
| | | | NOs: 31 and 32) |
| | AA670408 | 15q21-22.2 | β₂-microglobulin; MHC Class I |
| | | | (SEQ ID NOs: 33 and 34) |
| | N64862 | 5p14.1 | FYN-binding protein; T cell |
| | | | signaling (SEQ ID NOs: 35 |
| | | | and 36) |
| | R47979 | 6p21.3 | MHC class II DR α (SEQ ID |
| | | | NOs: 37 and 38) |
| Cancer/ | AA011215 | Xp22.1 | Spermidine/spermine N1- |
| Neoplasia | | | acetyltransferase; |
| | | | carcinogenesis (SEQ ID |
| | | | NOs: 39 and 40) |
| | AA496780 | 3q22.1 | RAS oncogene family member |
| | | | RAB7 (SEQ ID NOs: 41 and 42) |
| | AA609599 | Xp11.2-11.1 | Synovial sarcoma breakpoint |
| | | | 3 (SEQ ID NOs: 43 and 44) |
| | AA629897 | 3p21.3 | 67 kD laminin receptor |
| | | | expressed in colon |
| | | | carcinoma (SEQ ID NOs: 45 |
| | | | and 46) |
| | AA676470 | 17q21.31 | Ovarian carcinoma antigen; |
| | | | CA-125 (SEQ ID NOs: 47 |
| | | | and 48) |
| | H82419 | 20p13 | Protein tyrosine phosphatase; |
| | | | neoplastic transformation |
| | | | (SEQ ID NOs: 49 and 50) |
| | R45413 | 12q13-14 | Sarcoma amplified sequence |
| | | | (SEQ ID NOs: 51 and 52) |
| | W73144 | 19p13.3 | L-plastin; related to colon |
| | | | cancer metastasis (SEQ ID |
| | | | NOs: 53 and 54) |
| | W80637 | 7q11-21.3 | LIM and SHE protein-1; LASP- |
| | | | 1 (SEQ ID NOs: 55 and 56) |
| Growth | AA171463 | 5q23 | Sorting nexin 2; related to |
| Factors | | | EGF receptor (SEQ ID NOs: |
| | | | 57 and 58) |
| | AA424743 | 14q22-24 | EGF-response factor 1 (SEQ |
| | | | ID NOs: 59 and 60) |
| | AA490011 | 2p21-22 | Latent TGFβ binding protein 1 |
| | | | (SEQ ID NOs: 61 and 62) |
| Metab- | AA487466 | 2p25 | Ornithine decarboxylase |
| olism | | | antizyme 1 (SEQ ID NOs: 63 |
| | | | and 64) |
| | N21576 | 20q13.2-13.3 | Cytochrome P450 subfamily |
| | | | 24 (SEQ ID NOs: 65 and 66) |
| | T73294 | 7q11.23 | P450 cytochrome |
| | | | oxidoreductase (SEQ ID |
| | | | NOs: 67 and 68) |
| Cartilage/ | AA434115 | 1q31.1 | Chitinase 3-like; cartilage |
| Bone | | | glycoprotein-39 (SEQ ID |
| | | | NOs: 69 and 70) |
| | AA463225 | 14q22-23 | Bone morphogenetic protein 4 |
| | | | (SEQ ID NOs: 71 and 72) |
| Other | AA007419 | 1q23.1 | Regulator of G-protein |
| | | | signaling 4 (SEQ ID NOs: 73 |
| | | | and 74) |
| | N59636 | 16p13.3 | Hemoglobin zeta (SEQ ID |
| | | | NOs: 75 and 76) |
| | R43766 | 19pter-q12 | Eukaryotic translation |
| | | | elongation factor 2 (SEQ ID |
| | | | NOs: 77 and 78) |

The genes upregulated in established RA showed chromosomal clustering (see Table 6). Chromosome 1 included two clusters with a total of 5 upregulated genes and chromosomes 12 and 14 each included one cluster of 4 genes. Two upregulated genes, both related to MHC Class II proteins, were located on chromosome 6.

TABLE 6
Clusters of Genes Upregulated in Established vs. Early RA Patients

| Chromo- | | | |
|---|---|---|---|
| some | Location | Acc. No. | Description |
| 1 | p34 | AA599175 | Nuclease sensitive element binding |
| | | | protein (SEQ ID NOs: 25 and 26) |
| | p34.2 | R37953 | Adenylyl cyclase-associated |
| | | | protein (SEQ ID NOs: 79 and 80) |
| | q21 | AA424743 | Calpactin-1 (SEQ ID NOs: |
| | | | 81 and 82) |
| | q25.3 | W55964 | Actin-related protein subunit 5 |
| | | | (SEQ ID NOs: 83 and 84) |
| | q31.1 | AA434115 | Cartilage glycoprotein-39 (SEQ ID |
| | | | NOs: 69 and 70) |
| 12 | q12.3 | AA487426 | Rho GDP dissociation inhibitor |
| | | | (SEQ ID NOs: 85 and 86) |
| | q13 | AA422058 | Methyltransferase-like 1 (SEQ ID |
| | | | NOs: 87 and 88) |
| | q13-14.1 | R45413 | Sarcoma amplified sequence (SEQ |
| | | | ID NOs: 51 and 52) |
| | q24 | H73276 | Actin-related protein subunit 3 |
| | | | (SEQ ID NOs: 89 and 90) |
| 14 | q25.3 | AA424743 | EGF-response Factor 1 (SEQ ID |
| | | | NOs: 59 and 60) |
| | q21-24 | AA598526 | Hypoxia inducible factor (SEQ ID |
| | | | NOs: 91 and 92) |
| | q22-23 | AA463225 | Bone morphogenetic protein 4 |
| | | | (SEQ ID NOs: 71 and 72) |
| | q24.3-31 | H484982 | Checkpoint suppressor 1 (SEQ ID |
| | | | NOs: 93 and 94) |

### Example 6

### Fluorescent Labeling of Nucleic Acids

Examples 6-8 disclose a representative approach to preparing a nucleic acid-containing microarray and hybridizing labeled nucleic acids to the microarray. It should be understood that the approaches outlined in these Examples are exemplary only, and one of skill in the art will understand that variations to the specific approaches can be used without departing from the scope of the current disclosure.

A nucleic acid sample is used as a template for direct incorporation of fluorescent nucleotide analogs (e.g., Cy3-dUTP and Cy5-dUTP, available from Amersham Pharmacia Biotech of Piscataway, N.J., United States of America) by a randomly primed polymerization reaction. In brief, a 50 μl labeling reaction can contain 2 μg of template DNA, 5 μl of 10× buffer, 1.5 μl of fluorescent dUTP, 0.5 μl each of dATP, dCTP, and dGTP, 1 μl of random hexamers and decamers, and 2 μl of Klenow (E. coli DNA polymerase 3′ to 5′ exonuclease-minus from New England Biolabs of Beverly, Mass., United States of America).

### Example 7

### Noncovalent Binding of Nucleic Acid Probes onto Glass

PCR fragments derived from reference genes are suspended in a solution of 3 to 5M NaSCN and spotted onto amino-silanized slides using a GMS 417™ arrayer from Affymetrix of Santa Clara, Calif., United States of America. After spotting, the slides are heated at 80° C. for 2 hours to dehydrate the spots. Prior to hybridization, the slides are washed in isopropanol for 10 minutes, followed by washing in boiling water for 5 minutes. The washing steps remove any nucleic acid that is not bound tightly to the glass and help to reduce background created by redistribution of loosely attached DNA during hybridization. Contaminants such as detergents and carbohydrates should be minimized in the spotting solution. See also Maitra & Thakur, 1994 and Maitra & Thakur, 1992.

### Example 8

### Hybridization of Target Nucleic Acids and a Microarray

Labeled nucleic acids from the sample are prepared in a solution of 4×SSC buffer, 0.7 μg/μl tRNA, and 0.3% SDS to a total volume of 14.75 μl. The hybridization mixture is denatured at 98° C. for 2 minutes, cooled to 65° C., applied to the microarray, and covered with a 22-mm² cover slip. The slide is placed in a waterproof hybridization chamber for hybridization in a 65° C. water bath for 3 hours. Following hybridization, slides are washed in 1×SSC buffer with 0.06% SDS followed by 2 minutes in 0.06×SSC buffer.

### REFERENCES

The references listed below as well as all references cited in the specification are incorporated herein by reference to the extent that they supplement, explain, provide a background for, or teach methodology, techniques, and/or compositions employed herein.
Albert J, Wahlberg J, Lundeberg J, Cox S, Sandstrom E, Wahren B & Uhlen M (1992) Persistence of Azidothymidine-Resistant Human Immunodeficiency Virus Type 1 RNA Genotypes in Posttreatment Sera. J Virol 66:5627-5630.
Alexay C, Kain R C, Hanzel D K & Johnston R F (1996) Fluorescence scanner employing a macro scanning objective, in Menzel E R, ed, Fluorescence Detection IV. Proc SPIE 2705:63-72.
Altschul S F, Gish W, Miller W, Myers E W & Lipman D J (1990) Basic Local Alignment Search Tool. J Mol Biol 215:403-410.
Arnett F C, Edworthy S M, Bloch D A, McShane D J, Fries J F, Cooper N S, Healey L A, Kaplan S R, Liang M H, Luthra H S, et al. (1988) The American Rheumatism Association 1987 Revised Criteria for the Classification of Rheumatoid Arthritis, Arthritis Rheum 31:315-324.
Arunachalam B, Phan U T, Geuze H J & Cresswell P (2000) Enzymatic reduction of disulfide bonds in lysosomes: characterization of a gamma-interferon-inducible lysosomal thiol reductase (GILT). Proc Natl Acad Sci USA 97:745-750.
Ausubel F M, Brent R, Kingston R E, Moore D D, Seidman J G, Smith J A & Struhl K, eds (1994) Current Protocols in Molecular Biology. Wiley, New York, United States of America.
Baeten D, Boots A M, Steenbakkers P G, Elewaut D, Bos E, Verheijden G F, Berheijden G, Miltenburg A M, Rijnders A W, Veys E M et al. (2000) Human cartilage gp-39⁺,CD16⁺ monocytes in peripheral blood and synovium: correlation with joint destruction in rheumatoid arthritis. Arthritis Rheum 43:1233-1243.
Bej A K, Mahbubani M H, Dicesare J L & Atlas R M (1991) Polymerase Chain Reaction-Gene Probe Detection of Microorganisms by Using Filter-Concentrated Samples. Appl Environ Microbiol 57:3529-3534.
Boom R, Sol C J, Salimans M M, Jansen C L, Wertheim-van Dillen P M & van der Noordaa J (1990) Rapid and Simple Method for Purification of Nucleic Acids. J Clin Microbiol 28:495-503.
Buffone G J, Demmler G J, Schimbor C M & Greer J (1991) Improved Amplification of Cytomegalovirus DNA from Urine after Purification of DNA with Glass Beads. Clin Chem 37:1945-1949.
Busch M P, Wilber J C, Johnson P, Tobler L & Evans C S (1992) Impact of Specimen Handling and Storage on Detection of Hepatitis C Virus RNA. Transfusion 32:420-425.
Cha R S & Thilly W G (1993) Specificity, Efficiency, and Fidelity of Pcr. PCR Methods Appl 3:S18-29.
Chiodi F, Keys B, Albert J, Hagberg L, Lundeberg J, Uhlen M, Fenyo E M & Norkrans G (1992) Human Immunodeficiency Virus Type 1 Is Present in the Cerebrospinal Fluid of a Majority of Infected Individuals. J Clin Microbiol 30:1768-1771.
Davies D E, Polosa R, Puddicombe S M, Richter A & Holgate S T (1999) The epidermal growth factor receptor and its ligand family: their potential role in repair and remodelling in asthma. Allergy 54:771-783.
DeRisi J, Penland L, Brown P O, Bittner M L, Meltzer P S, Ray M, Chen Y, Su Y A & Trent J M (1996) Use of a cDNA Microarray to Analyse Gene Expression Patterns in Human Cancer. Nat. Genet 14:457-460.
Didier D K, Schiffenbauer J, Woulfe S L, Zacheis M & Schwartz B D (1988) Characterization of the cDNA encoding a protein binding to the major histocompatibility complex class II Y box. Proc Natl Acad Sci USA 85:7322-7326.
Dubiley S, Kirillov E, Lysov Y & Mirzabekov A (1997) Fractionation, Phosphorylation and Ligation on Oligonucleotide Microchips to Enhance Sequencing by Hybridization. Nucleic Acids Res 25:2259-2265.
Eberwine J, Yeh H, Miyashiro K, Cao Y, Nair S, Funnell R, Zeftel M & Coleman P (1992) Analysis of Gene Expression in Single Live Neurons. Proc Natl Acad Sci USA 89:3010-3014.
Eisen M B, Spellman P T, Brown P O & Botstein D (1998) Cluster Analysis and Display of Genome-Wide Expression Patterns. Proc Natl Acad Sci U S A 95:14863-14868.
Englert D (2000) in Schena M, ed, Microarray Biochip Technology, pp. 231-246, Eaton Publishing, Natick, Mass., United States of America.
Fodor S P, Read J L, Pirrung M C, Stryer L, Lu A T & Solas D (1991) Light-Directed, Spatially Addressable Parallel Chemical Synthesis. Science 251:767-773.
Fodor S P, Rava R P, Huang X C, Pease A C, Holmes C P & Adams C L (1993) Multiplexed Biochemical Assays with Biological Chips. Nature 364:555-556.
Guedon P, Livache T, Martin F, Lesbre F, Roget A, Bidan G & Levy Y (2000) Characterization and Optimization of a Real-Time, Parallel, Label-Free, Polypyrrole-Based DNA Sensor by Surface Plasmon Resonance Imaging. Anal Chem 72:6003-6009.
Hamel A L, Wasylyshen M D & Nayar G P (1995) Rapid Detection of Bovine Viral Diarrhea Virus by Using RNA Extracted Directly from Assorted Specimens and a One-Tube Reverse Transcription Pcr Assay. J Clin Microbiol 33:287-291.
Heaton R J, Peterson A W & Georgiadis R M (2001) Electrostatic Surface Plasmon Resonance Direct Electric Field-Induced Hybridization and Denaturation in Monolayer Nucleic Acid Films and Label-Free Discrimination of Base Mismatches. Proc Natl Acad Sci USA 98:3701-3704.
Henikoff S & Henikoff J G (1992) Amino Acid Substitution Matrices from Protein Blocks. Proc Natl Acad Sci U S A 89:10915-10919.
Hermanson G T (1990) Bioconjugate Techniques, Academic Press, San Diego, Calif., United States of America.
Herrewegh A A, de Groot R J, Cepica A, Egberink H F, Horzinek M C & Rottier P J (1995) Detection of Feline Coronavirus RNA in Feces, Tissues, and Body Fluids of Naturally Infected Cats by Reverse Transcriptase Pcr. J Clin Microbiol 33:684-689.
Izraeli S, Pfleiderer C & Lion T (1991) Detection of Gene Expression by Pcr Amplification of RNA Derived from Frozen Heparinized Whole Blood. Nucleic Acids Res 19:6051.
Jacobson D L, Gange S J, Rose N R & Graham N M (1997) Epidemiology and Estimated Population Burden of Selected Autoimmune Diseases in the United States. Clin Immunol Immunopathol 84:223-243.
Joyce C (2002) Quantitative RT-PCR. A Review of Current Methodologies. Methods Mol Biol 193:83-92.
Karlin S & Altschul S F (1993) Applications and Statistics for Multiple High-Scoring Segments in Molecular Sequences. Proc Natl Acad Sci USA 90:5873-5877.
Kim S, Dougherty E R, Chen Y, Sivakumar K, Meltzer P, Trent J M & Biftner M (2000) Multivariate Measurement of Gene Expression Relationships. Genomics 67:201-209.
Kohsaka H & Carson DA (1994) Solid-Phase Polymerase Chain Reaction. J Clin Lab Anal 8:452-455.
Kotzin B L (1996) Systemic Lupus Erythematosus. Cell 85:303-306.
Krichevsky A M, Metzer E & Rosen H (1999) Translational Control of Specific Genes During Differentiation of HI-60 Cells. J Biol Chem 274:14295-14305.
Kukreja A & Maclaren N K (2000) Current Cases in Which Epitope Mimicry Is Considered as a Component Cause of Autoimmune Disease: Immune-Mediated (Type 1) Diabetes. Cell Mol Life Sci 57:534-541.
Lanciotti R S, Calisher C H, Gubler D J, Chang G J & Vorndam A V (1992) Rapid Detection and Typing of Dengue Viruses from Clinical Samples by Using Reverse Transcriptase-Polymerase Chain Reaction. J Clin Microbiol 30:545-551.
Leong L M & Brickell P M (1996) Bone morphogenic protein-4. Int J. Biochem. Cell Biol. 28:1293-1296.
Leveen P, Larsson J, Ehinger M, Cilio C M, Sundler M, Sjostrand L J, Holmdahl R & Karlsson S (2002) Induced disruption of the transforming growth factor beta type II receptor gene in mice causes a lethal inflammatory disorder that is transplantable. Blood 100:560-568.
Linz U, Delling U & Rubsamen-Waigmann H (1990) Systematic Studies on Parameters Influencing the Performance of the Polymerase Chain Reaction. J Clin Chem Clin Biochem 28:5-13.
Lisle C M, Bortolin S, Benight A S, Janeczko R A & Zastawny R L (2001) Novel Signal Amplification Technology with Applications in DNA and Protein Detection Systems. Biotechniques 30:1268-1272.
Liu J & Hlady V (1996) Chemical pattern on silica surface prepared by UV irradiation of 3-mercapto-propyltriethoxy silane layer: Surface characterization and fibrinogen adsorption. Colloids and Surfaces B. Biointerfaces 8:25-37.
Maas K, Chan S, Parker J, Slater A, Moore J, Olsen N & Aune T M (2002) Cutting edge: molecular portrait of human autoimmune disease. J Immunol 169:5-9.
Mace M L, Jr., Montagu J, Rose S D & McGuinness G (2000) in Schena M ed, Microarray Biochip Technology, pp. 39-64, Eaton Publishing, Natick, Mass., United States of America
Maier E, Meier-Ewert S, Ahmadi A R, Curtis J & Lehrach H (1994) Application of Robotic Technology to Automated Sequence Fingerprint Analysis by Oligonucleotide Hybridisation. J Biotechnol 35:191-203.
Maitra R & Thakur A R (1992) Curr Sci 62:586-588.
Maitra R & Thakur A R (1994) Multiple Fragment Ligation on Glass Surface: A Novel Approach. Indian J Biochem Biophys 31:97-99.
Marrack P. Kappler J & Kotzin B L (2001) Autoimmune Disease: Why and Where It Occurs. Nat. Med 7:899-905.
Martin A, Barbesino G & Davies T F (1999) T-Cell Receptors and Autoimmune Thyroid Disease—Signposts for T-Cell-Antigen Driven Diseases. Int Rev Immunol 18:111-140.
McCaustland K A, Bi S, Purdy M A & Bradley D W (1991) Application of Two RNA Extraction Methods Prior to Amplification of Hepatitis E Virus Nucleic Acid by the Polymerase Chain Reaction. J Virol Methods 35:331-342.
McPherson M J, Hames B D & Taylor G, eds, (1995) PCR 2: A Practical Approach, IRL Press, New York, N.Y., United States of America.
Mendelsohn J & Baselga J (2000) The EGF receptor family as targets for cancer therapy. Oncogene 19:6550-6565.
Millar D S, Withey S J, Tizard M L, Ford J G & Hermon-Taylor J (1995) Solid-Phase Hybridization Capture of Low-Abundance Target DNA Sequences: Application to the Polymerase Chain Reaction Detection of Mycobacterium Paratuberculosis and Mycobacterium Avium Subsp. Silvaticum. Anal Biochem 226:325-330.
Natarajan V, Plishka R J, Scott E W, Lane H C & Salzman N P (1994) An Internally Controlled Virion Pcr for the Measurement of Hiv-1 RNA in Plasma. PCR Methods Appl 3:346-350.
Needleman S B & Wunsch C D (1970) A General Method Applicable to the Search for Similarities in the Amino Acid Sequence of Two Proteins. J Mol Biol 48:443-453.
Nelson B P, Grimsrud T E, Liles M R, Goodman R M & Corn R M (2001) Surface Plasmon Resonance Imaging Measurements of DNA and RNA Hybridization Adsorption onto DNA Microarrays. Anal Chem 73:1-7.
O'Donnell M J, Tang K, Köster H, Smith C L & Cantor C R (1997) High-Density, Covalent Attachment of DNA to Silicon Wafers for Analysis by MALDI-TOF Mass Spectrometry. Anal Chem 69:2438-2443.

Paladichuk A (1999) Isolating RNA: Pure and Simple. The Scientist 13(16):20-23.
PCT International Publication No. WO 97/14028.
PCT International Publication No. WO 99/19515
PCT International Publication No. WO 99/63385
PCT International Publication No. WO 01/13120
PCT International Publication No. WO 01/14589
PCT International Publication No. WO 01/23082
Pearson W R & Lipman D J (1988) Improved Tools for Biological Sequence Comparison. Proc Natl Acad Sci USA 85:2444-2448.
Pietu G, Alibert O, Guichard V, Lamy B, Bois F, Leroy E, Mariage-Sampson R, Houlgatte R, Soularue P & Auffray C (1996) Novel Gene Transcripts Preferentially Expressed in Human Muscles Revealed by Quantitative Hybridization of a High Density Cdna Array. Genome Res 6:492-503.
Quayle A J, Wilson K B, Li S G, Kjeldsen-Kragh J, Oftung F, Shinnick T, Sioud M, Forre O, Capra J D & Natvig J B (1992) Peptide Recognition, T Cell Receptor Usage and HIa Restriction Elements of Human Heat-Shock Protein (Hsp) 60 and Mycobacterial 65-Kda Hsp-Reactive T Cell Clones from Rheumatoid Synovial Fluid. Eur J Immunol 22:1315-1322.
Randolph J B & Waggoner A S (1997) Stability, Specificity and Fluorescence Brightness of Multiply-Labeled Fluorescent DNA Probes. Nucleic Acids Res 25:2923-2929.
Ratner B D & Castner D G (1997) in Vickerman J C, ed, Surface Analysis: The Principal Techniques, John Wiley & Sons, New York, N.Y., United States of America.
Robertson J M & Walsh-Weller J (1998) An Introduction to Pcr Primer Design and Optimization of Amplification Reactions. Methods Mol Biol 98:121-154.
Rose D (2000) in Schena M ed, Microarray Biochip Technology, pp. 19-38, Eaton Publishing, Natick, Mass., United States of America.
Roux K H (1995) Optimization and Troubleshooting in Pcr. PCR Methods Appl 4:S185-194.
Rupp G M & Locker J (1988) Purification and Analysis of RNA from Paraffin-Embedded Tissues. Biotechniques 6:56-60.
Saevarsdottir S, Vikingsdoftir T, Vikingsson A, Manfredsdottir V, Geirsson A J & Valdimarsson H (2001) Low mannose binding lectin predicts poor prognosis in patients with early rheumatoid arthritis. A prospective study. J Rheumatol 28:728-34.
Sambrook & Russell (2001) Molecular Cloning: A Laboratory Manual, 3^{rd} Edition, Cold Spring Harbor Laboratory Press, Cold Spring Harbor, N.Y., United States of America.
Sapolsky R J & Lipshutz R J (1996) Mapping Genomic Library Clones Using Oligonucleotide Arrays. Genomics 33:445-456.
Schena M, Shalon D, Davis R W & Brown P O (1995) Quantitative Monitoring of Gene Expression Patterns with a Complementary DNA Microarray. Science 270:467-470.
Schena M, Shalon D, Heller R, Chai A, Brown PO & Davis R W (1996) Parallel Human Genome Analysis: Microarray-Based Expression Monitoring of 1000 Genes. Proc Natl Acad Sci USA 93:10614-10619.
Shalon D, Smith S J & Brown P O (1996) A DNA Microarray System for Analyzing Complex DNA Samples Using Two-Color Fluorescent Probe Hybridization. Genome Res 6:639-645.
Sherlock G (2000) Analysis of Large-Scale Gene Expression Data. Curr Opin Immunol 12:201-205.
Shoemaker D D, Lashkari D A, Morris D, Mittmann M & Davis R W (1996) Quantitative Phenotypic Analysis of Yeast Deletion Mutants Using a Highly Parallel Molecular Bar-Coding Strategy. Nat. Genet 14:450-456.
Shriver-Lake L C (1998) in Cass T & Ligler F S, eds, Immobilized Biomolecules in Analysis, pp. 1-14, Oxford Press, Oxford, United Kingdom.
Smith P L, WalkerPeach C R, Fulton R J & DuBois D B (1998) A Rapid, Sensitive, Multiplexed Assay for Detection of Viral Nucleic Acids Using the Flowmetrix System. Clin Chem 44:2054-2056.
Smith T F & Waterman M (1981) Comparison of Biosequences. Adv Appl Math 2:482-489.
Southern E M (1975) Detection of Specific Sequences among DNA Fragments Separated by Gel Electrophoresis. J Mol Biol 98:503-517.
Steel A, Torres M, Hartwell J, Yu Y Y, Ting N, Hoke G & Yang, H (2000) in Schena M, ed, Microarray Biochip Technology, pp. 87-118, Eaton Publishing, Natick, Mass., United States of America.
Strain S R & Chmielewski J G (2001) ROCK: A Spreadsheet-Based Program for the Generation and Analysis of Random Oligonucleotide Primers used in PCR. BioTechniques 30:1286-1293.
Tanaka S, Minagawa H, Toh Y, Liu Y & Mori R (1994) Analysis by RNA-Pcr of Latency and Reactivation of Herpes Simplex Virus in Multiple Neuronal Tissues. J Gen Virol 75 (Pt 10):2691-2698.
Telenius H, Carter N P, Bebb C E, Nordenskjold M, Ponder B A & Tunnacliffe A (1992) Degenerate Oligonucleotide-Primed Pcr: General Amplification of Target DNA by a Single Degenerate Primer. Genomics 13:718-725.
Theriault T P, Winder S C & Gamble R C (1999) in Schena M, ed, DNA Microarrays: A Practical Approach, pp. 101-120, Oxford University Press Inc., New York, N.Y., United States of America.
Tijssen P (1993) Laboratory Techniques in Biochemistry and Molecular Biology-Hybridization with Nucleic Acid Probes. Elsevier, New York.
Ufret-Vincenty R L, Quigley L, Tresser N, Pak S H, Gado A, Hausmann S, Wucherpfennig K W & Brocke S (1998) In Vivo Survival of Viral Antigen-Specific T Cells That Induce Experimental Autoimmune Encephalomyelitis. J Exp Med 188:1725-1738.
U.S. Pat. No. 4,729,947
U.S. Pat. No. 5,346,603
U.S. Pat. No. 5,445,934
U.S. Pat. No. 5,207,880
U.S. Pat. No. 5,230,781
U.S. Pat. No. 5,360,523
U.S. Pat. No. 5,534,125
U.S. Pat. No. 5,571,388
U.S. Pat. No. 5,743,960
U.S. Pat. No. 5,843,767
U.S. Pat. No. 5,846,717
U.S. Pat. No. 5,916,524
U.S. Pat. No. 5,965,352
U.S. Pat. No. 5,985,557
U.S. Pat. No. 5,994,069
U.S. Pat. No. 6,001,567
U.S. Pat. No. 6,066,457
U.S. Pat. No. 6,090,543
U.S. Pat. No. 6,017,696
U.S. Pat. No. 6,086,737
U.S. Pat. No. 6,123,819
U.S. Pat. No. 6,162,603
U.S. Pat. No. 6,225,059
U.S. Pat. No. 6,245,508
Vandesompele J, De Preter K, Pattyn F, Poppe B, Van Roy N, De Paepe A & Speleman F (2002) Acurate Normalization of Real-Time Quantitative RT-PCR Data by Geometric Averaging of Multiple Internal Control Genes. Genome Biol 3:1-12.
Van Gelder R N, von Zastrow M E, Yool A, Dement W C, Barchas J D & Eberwine J H (1990) Amplified RNA Synthesized from Limited Quantities of Heterogeneous cDNA. Proc Natl Acad Sci USA 87:1663-1667.
Van Kerckhoven I, Fransen K, Peeters M, De Beenhouwer H, Piot P & van der Groen G (1994) Quantification of Human Immunodeficiency Virus in Plasma by RNA Pcr, Viral Culture, and P24 Antigen Detection. J Clin Microbiol 32:1669-1673.
Vignali D A (2000) Multiplexed Particle-Based Flow Cytometric Assays. J Immunol Methods 243:243-255.
Wang A M, Doyle M V & Mark D F (1989) Quantitation of Mma by the Polymerase Chain Reaction. Proc Natl Acad Sci USA 86:9717-9721.
Wang E, Miller L D, Ohnmacht G A, Liu E T & Marincola F M (2000) High-Fidelity Mrna Amplification for Gene Profiling. Nat. Biotechnol 18:457-459.
Warrington J A, Dee S & Trulson M (2000) in Schena M, ed, Microarray Biochip Technology, pp. 119-148, Eaton Publishing, Natick, Mass., United States of America.
Williams J F (1989) Optimization Strategies for the Polymerase Chain Reaction. Biotechniques 7:762-769.
Williams J G, Kubelik A R, Livak K J, Rafalski J A & Tingey S V (1990) DNA Polymorphisms Amplified by Arbitrary Primers Are Useful as Genetic Markers. Nucleic Acids Res 18:6531-6535.
Worley J et al. (2000) in Schena M, ed, Microarray Biochip Technology, pp. 65-86, Eaton Publishing, Natick, Mass., United States of America,
Yang P, Deng T, Zhao D, Feng P, Pine D, Chmelka B F, Whitesides G M & Stucky G D (1998) Hierarchically Ordered Oxides. Science 282:2244-2246.
Yershov G, Barsky V, Belgovskiy A, Kirillov E, Kreindlin E, Ivanov I, Parinov S, Guschin D, Drobishev A, Dubiley S & Mirzabekov A (1996) DNA Analysis and Diagnostics on Oligonucleotide Microchips. Proc Natl Acad Sci USA 93:4913-4918.

It will be understood that various details of the claimed subject matter can be changed without departing from the scope of the claimed subject matter. Furthermore, the foregoing description is for the purpose of illustration only, and not for the purpose of limitation.

What is claimed is:

1. A method for detecting a predisposition to developing established rheumatoid arthritis (RA) in a subject, the method comprising:
(a) obtaining a biological sample from the subject;
(b) determining expression levels of at least two genes in the biological sample; and
(c) comparing the expression levels of each of the at least two genes determined in step (b) with a standard, wherein the comparing detects the predisposition to developing established rheumatoid arthritis in the subject.

2. The method of claim 1, wherein the biological sample is a cell.

3. The method of claim 3, wherein the cell is a peripheral blood mononuclear cell.

4. The method of claim 1, wherein the subject is an animal.

5. The method of claim 4, wherein the animal is a mammal.

6. The method of claim 5, wherein the mammal is a human.

7. The method of claim 1, wherein the determining comprises a technique selected from the group consisting of a Northern blot, hybridization to a nucleic acid microarray, and a reverse transcription-polymerase chain reaction (RT-PCR).

8. The method of claim 7, wherein the RT-PCR is quantitative RT-PCR.

9. The method of claim 1, wherein the determining is of the expression levels of at least two genes represented by SEQ ID NOs: 1-94.

10. The method of claim 9, wherein the determining is of the expression levels of at least five genes represented by SEQ ID NOs: 1-94.

11. The method of claim 9, wherein the determining is of the expression levels of at least ten genes represented by SEQ ID NOs: 1-94.

12. The method of claim 9, wherein the determining is of the expression levels of at least twenty genes represented by SEQ ID NOs: 1-94.

13. The method of claim 9, wherein the determining is of the expression levels of at least twenty-five genes represented by SEQ ID NOs: 1-94.

14. The method of claim 9, wherein the determining is of the expression levels of all of the genes represented by SEQ ID NOs: 1-94.

15. The method of claim 1, wherein the comparing comprises:
(a) establishing an average expression level for each of the at least two genes in a population, wherein the population comprises statistically significant numbers of subjects with early rheumatoid arthritis (RA) and subjects that have established RA;
(b) assigning a first value to each gene for which the expression level in the subject is higher than the average expression level in the population and a second value to each gene for which the expression level in the subject is lower than the average expression level in the population; and
(c) adding the values assigned in step (b) to arrive at a sum, wherein the sum is indicative of the predisposition of the subject to develop established RA.

16. A method for facilitating a diagnosis of rheumatoid arthritis (RA) in a subject, the method comprising:
(a) providing an array comprising a plurality of nucleic acid sequences, wherein each nucleic acid sequence corresponds to a reference gene;
(b) providing a biological sample derived from the subject, wherein the biological sample comprises a nucleic acid;
(c) hybridizing the biological sample to the array;
(d) detecting all nucleic acids on the array to which the biological sample hybridizes;
(e) determining an expression level for each nucleic acid detected;
(f) creating a profile of the expression levels for the detected nucleic acids; and
(g) comparing the profile created with a standard profile, wherein the comparing facilitates a diagnosis of rheumatoid arthritis (RA) in the subject.

17. The method of claim 16, wherein the array is selected from the group consisting of a microarray chip and a membrane-based filter array.

18. The method of claim 17, wherein the array comprises nucleic acid sequences corresponding to at least two genes represented by SEQ ID NOs: 1-94.

19. The method of claim 17, wherein the array comprises nucleic acid sequences corresponding to at least five genes represented by SEQ ID NOs: 1-94.

20. The method of claim 17, wherein the array comprises nucleic acid sequences corresponding to at least ten genes represented by SEQ ID NOs: 1-94.

21. The method of claim 17, wherein the array comprises nucleic acid sequences corresponding to at least twenty genes represented by SEQ ID NOs: 1-94.

22. The method of claim 17, wherein the array comprises nucleic acid sequences corresponding to at least twenty-five genes represented by SEQ ID NOs: 1-94.

23. The method of claim 17, wherein the array comprises nucleic acid sequences corresponding to all of the genes represented by SEQ ID NOs: 1-94.

24. The method of claim 17, wherein the array further comprises nucleic acid sequences corresponding to at least one internal control gene.

25. The method of claim 16, wherein the biological sample is a cell.

26. The method of claim 25, wherein the cell is a peripheral blood mononuclear cell.

27. The method of claim 16, wherein the subject is an animal.

28. The method of claim 27, wherein the animal is a mammal.

29. The method of claim 28, wherein the mammal is a human.

30. The method of claim 16, wherein the determining comprises a technique selected from the group consisting of a Northern blot, hybridization to a nucleic acid microarray, and a reverse transcription-polymerase chain reaction (RT-PCR).

31. The method of claim 30, wherein the RT-PCR is quantitative RT-PCR.

32. The method of claim 16, wherein the determining is of the expression levels of at least two genes represented by SEQ ID NOs: 1-94.

33. The method of claim 32, wherein the determining is of the expression levels of at least five genes represented by SEQ ID NOs: 1-94.

34. The method of claim 33, wherein the determining is of the expression levels of the eight genes represented by SEQ ID NOs: 3-18.

35. The method of claim 32, wherein the determining is of the expression levels of at least ten genes represented by SEQ ID NOs: 1-94.

36. The method of claim 34, wherein the determining is of the expression levels of the ten genes represented by SEQ ID NOs: 33, 34, 37-40, 43, 44, 51, 52, 69, 70, 73-78, 93, and 94.

37. The method of claim 32, wherein the determining is of the expression levels of all of the genes represented by SEQ ID NOs: 1-94.

38. The method of claim 16, wherein the determining an expression level for each nucleic acid detected further comprises normalizing the expression level that is determined for each nucleic acid detected relative to an expression level of another gene present on the array, wherein the another gene present on the array is a gene for which the expression level does not vary in the population.

39. The method of claim 16, wherein the comparing comprises:
(a) establishing an average expression level for each gene in a population, wherein the population comprises statistically significant numbers of subjects with early rheumatoid arthritis (RA) and subjects that have established RA;
(b) assigning a first value to each gene for which the expression level in the subject is higher than the average expression level in the population and a second value to each gene for which the expression level in the subject is lower than the average expression level in the population; and
(c) adding the values assigned in step (b) to arrive at a sum, wherein the sum is indicative of the predisposition of the subject to develop established RA.

40. A kit comprising a plurality of oligonucleotide primers and instructions for employing the plurality of oligonucleotide primers to determine the expression level of at least one of the genes represented by SEQ ID NOs: 1-94.

41. The kit of claim 40, comprising oligonucleotide primers to determine the expression level of at least five of the genes represented by SEQ ID NOs: 1-94.

42. The kit of claim 40, comprising oligonucleotide primers to determine the expression level of at least ten of the genes represented by SEQ ID NOs: 1-94.

43. The kit of claim 40, comprising oligonucleotide primers to determine the expression level of at least twenty of the genes represented by SEQ ID NOs: 1-94.

44. The kit of claim 40, comprising oligonucleotide primers to determine the expression level of at least thirty of the genes represented by SEQ ID NOs: 1-94.

45. The kit of claim 40, comprising oligonucleotide primers to determine the expression level of at all of the genes represented by SEQ ID NOs: 1-94.

46. The kit of claim 40, further comprising oligonucleotide primers to determine the expression level of a control gene.
